SEQUENCE LISTING (124 sequences)

SEQ ID NO: 1 - DNA, 7969 bp, synthetic construct
  misc_feature 1..7969: note = Codon Optimized
  source 1..7969: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgactcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccatactagttctagatccgtcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttaaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgccctgttctctgtagttgcgctaagagaatggacctatgaactgatggttggtgaagaaaacaatattttggtgctgggattctttttttttctggatgccagcttaaaaagcgggctccattatatttagtggatgccaggaataaacctgttcacccaagcaccatcagtgttatatattctgtgtaacccgccccctattttggcatgtacgggttacagcagaattaaaaggctaattttttgactaaataaagttaggaaaatcactactattaattatttacgtattctttgaaatggcagtattgataatgataaactcgagagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 2 - DNA, 10004 bp, synthetic construct
  misc_feature 1..10004: note = Codon Optimized
  source 1..10004: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctttcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaatgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgacttacttgtcatcgtcatctttgtagtcaatatcgtggtctttatagtcaccgtcatgatccttgtaatcctttgatctcttgtagaccttattcaagaaagctgtcaaaacgtcggctttaaaacctcttgattcatcaacttgactaatctttgcctttaagtctttagcgatggattcttcgtattcatggtacaattgttcaatcttcaaatcattaaaaattttcttacactttgcttcagcaactgagtcctttttaccgtagttttcatccaaagtctttctttgttcggcagatgctaattccaaagccttgttaataacccaactgcacttattgtcttgaatatctgtaccgattttacctatttgttctggagtaccgaaacagtctaagtagtcatcttggatttggaagtattcacccaaaggtatcaaaacatctcttgcttgcttcaagtctttttcatcagtaataccagctacgtacatagccaaggcgactggcaaatagaaggagtaataagcagtttcaaaggtgacgatgaatgaatgtttcttcaaggaaaactttgacaagtcaactttatcttcaggtgcagttatcaaatccatcaattgacccaattctgtttggaaagtaacttcgtggaataattcggtaatatcgatgtagtacttttcgtttctgaaatgtgacttcaacaatttatagatagcggcttccaacataaaagcatcatttatggctatttcaccaacttctggaactttgtaccagcatggttgacctcttcttgttatagacttatccatcatgtcatcggcaaccaaaaagtatgcttgcaacaattcaatacaccaacccaagatagcgaccttttcgtattcttcttgacctaattgttcaacggttttgttagacaagatagcataagtatcaactacactcaaacctctattcaatttaccacctggagtattgtagtttaaagagtgagcataccaatcgcaggcttctttaggcataccataagctaacaaactagcgttcaattcttcaactaactttgggaatacgttcaagaatctttctcttcttatttccttttctgaagccataggacctggattttcttcaacgtcaccacatgttaacaaagaacctctaccttcttcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccatactagttctagatccgtcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttaaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgccctgttctctgtagttgcgctaagagaatggacctatgaactgatggttggtgaagaaaacaatattttggtgctgggattctttttttttctggatgccagcttaaaaagcgggctccattatatttagtggatgccaggaataaacctgttcacccaagcaccatcagtgttatatattctgtgtaacccgccccctattttggcatgtacgggttacagcagaattaaaaggctaattttttgactaaataaagttaggaaaatcactactattaattatttacgtattctttgaaatggcagtattgataatgataaactcgagagctccagcttttgttcagttgattgtatgcttggtatagcttgaaatattgtgcagaaaaagaaacaaggaagaaagggaacgagaacaatgacgaggaaacaaaagattaataattgcaggtctatttatacttgatagcaagacagcaaacttttttttatttcaaattcaagtaactggaaggaaggccgtataccgttgctcattagagagtagtgtgcgtgaatgaaggaaggaaaaagtttcgtgtgcttcgagatacccctcatcagctctggaacaacgacatctgttggtgctgtctttgtcgttaattttttcctttagtgtcttccatcatttttttgtcattgcggatatggtgagacaacaacgggggagagagaaaagaaaaaaaaagaaaagaagttgcatgcgcctattattacttcaatagatggcaaatggaaaaagggtagtgaaacttcgatatgatgatggctatcaagtctagggctacagtattagttcgttatgtaccaccatcaatgaggcagtgtaattggtgtagtcttgtttagcccattatgtcttgtctggtatctgttctattgtatatctcccctccgccacctacatgttagggagaccaacgaaggtattataggaatcccgatgtatgggtttggttgccagaaaagaggaagtccatattgtacacccggaaacaacaaaaggatgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 3 - DNA, 9508 bp, synthetic construct
  misc_feature 1..9508: note = Codon Optimized
  source 1..9508: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaatagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgacttacaaatcttcttcacttattaatttttgttcgtggtggtgaggtggcaaaggtgggatggattgttcgtttctgaaaaagttgttagggtcggctttagtctttactttaactaatctgttgaaatttttaccaaagtacttttcaccccaaattcttgcttgtgtatagttatttggagattcagggttagttttacctaagtccaaatctctgtagttcaaatatgccaatcttgggttttgactaacgtaaggtgtagtgaagttgtaaacggatctgacccagttgatatgcttttcgttatcttcttgcttttcccatgaggctgtgtaccataattcatacatgataccagctctgtgaggaaatggtatggctgattcagatatttcttccataataccaccgtatggatacaaaacgtacatgccgacacctacatcttcttcgtacaacttttccaatatcttgaccattgcagtttcagggattggtttcttaacgtagtccaatttaatagaaaaagcggtctttttaccagcggatctatccaacaagatttcctttttgaagttagcggtgttgaagtttacaacacctgaatagaagatggttgtgtctatccaagaaaattccttgcaatctgtctttttaatacccaattctgggaatgacttattcatcaaatcaaccaaagaatctacaccaccatggaaaattgaagaaaaataaccgtgaacagtggtcttatttttaccatggttatctgtaatatttttagtgatgaaatgggtcatcaaaaccaagtccttatcgtacttgtaagcgatgttttgccacttattaaacaacttaaccaaaccgtggatttccatgttctttttgacagagaaaatagtacttttggaaggaacagcgactaacttaattttccaagcggcaatgataccgaaattttcaccaccaccacctcttatggcccaaaacaaatcttcacccatagactttctgtccaaaactttaccatctacgttaaccaaatgggcgtctataatattatctgcagctaaaccgtagtttctcatcaatgcaccataaccaccaccagaaaagtgaccaccgacacctactgttggacagtaaccaccagggaaagaaaagttttcattcttttcgttgatccagtagtaaacttcacccaaggtggcacctgcttctacccatgctgtttgactgtgaacgtcgatctttatggaatgcatatttctcaaatcgactacaacgaatggaacttgtgagatgtaagacatgccttctgcatcatgaccacctgatctagttctgatttgcaaacctactttcttagagcacaatatagaagcttgaatgtgactaacattggaaggtgtaacaatgactaaaggttttggtgtagtgtcagaagtgaatctcaaattttggatggtactgttcaaaacggacatgtacaattgatcatgttgagtatatataaactttgggttagcagggttgtttgggatgtattcggagaaacacttcaaaaagttttcttgtggatttgcgatggagatttggatgttgaaggacaagaagaagaagattattttacagacgaaccagaaagagaatgcggagcagttcataggacctggattttcttcaacgtcaccacaggtcaacaaagaacctctaccttcaataaaaacgtataccaaatattcagcgtagtacaatttccacataaactcgtagaatcttctacctgcttcagggtcataatttgtcaaagcgaaatctctagtttgcaagatcaaccagaaagccaagatggcatgtgacaacaacataacgttagaattaaaggcttgtggccaaatgatacctgccaaaatggctgcgacgtaacttaacaaaacgataccggagcagaacaaagtcaaatttcttgaaccgtacttagaagccaaggtactaataccgaactttgtgtcaccttcaacgtcagaggcatccttgatcaaggctaatgcagaacccatacttttcatgaatgccaacaaaaatgtgaatgaaggtctcaattcgaatggcaaacctaaagcagctcttgaagcgtagtagaaggtgaagtttgtgatgatatgagctaagaaattcaacaaaaaggcagtactagggttttgtttccatctaaaaggtggtacggaatagacaataccaccgaagataccgaaacagtaaccgaagatgtacaatggaccacccttcattttaattgtgatgatcaaaccgaacaaggctactatgatagacatgatccatgcagtattgacggatatttcacctgaagccaaaggcaaatctggtttgttaattctgtcgatgtgcaaatcgtatatttgattaattgtagtggtgaatgaagcgatgcacaagatggcaactaaaaagaaaaatgccttgaacatcaaggaccatgaaattaagttagtgttatgcaacaattctttaccgaataaaccgcatgcacaagaagtaaaagcgattatggtgtatggtctttgcaacttccaacatgctttaccgaagttcaaaatttttgtggcaacagagtgattatcactttcaggtggttcagtttgatttgtagttgcagctctgatagagttcttagctatagacaaactttcggagcacttattttgtaagtggaaggacttggttgaacaatgttttgatggaaagttgttgtaagagtacttaataggtgtctttggatgtctgtaacacaacaatgatgtttttggattgttgttgtgaggattcaataaggtatgatagttagtttggaaggagaaagtacagacggatgataaacccatactagttctagatccgtcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttaaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgccctgttctctgtagttgcgctaagagaatggacctatgaactgatggttggtgaagaaaacaatattttggtgctgggattctttttttttctggatgccagcttaaaaagcgggctccattatatttagtggatgccaggaataaacctgttcacccaagcaccatcagtgttatatattctgtgtaacccgccccctattttggcatgtacgggttacagcagaattaaaaggctaattttttgactaaataaagttaggaaaatcactactattaattatttacgtattctttgaaatggcagtattgataatgataaactcgagagctccagcttttgttcagttgattgtatgcttggtatagcttgaaatattgtgcagaaaaagaaacaaggaagaaagggaacgagaacaatgacgaggaaacaaaagattaataattgcaggtctatttatacttgatagcaagacagcaaacttttttttatttcaaattcaagtaactggaaggaaggccgtataccgttgctcattagagagtagtgtgcgtgaatgaaggaaggaaaaagtttcgtgtgcttcgagatacccctcatcagctctggaacaacgacatctgttggtgctgtctttgtcgttaattttttcctttagtgtcttccatcatttttttgtcattgcggatatggtgagacaacaacgggggagagagaaaagaaaaaaaaagaaaagaagttgcatgcgcctattattacttcaatagatggcaaatggaaaaagggtagtgaaacttcgatatgatgatggctatcaagtctagggctacagtattagttcgttatgtaccaccatcaatgaggcagtgtaattggtgtagtcttgtttagcccattatgtcttgtctggtatctgttctattgtatatctcccctccgccacctacatgttagggagaccaacgaaggtattataggaatcccgatgtatgggtttggttgccagaaaagaggaagtccatattgtacacccggaaacaacaaaaggatgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 4 - DNA, 9505 bp, synthetic construct
  misc_feature 1..9505: note = Codon Optimized
  source 1..9505: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaatagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgacttacaaatcttcttcacttattaatttttgttcgtgtctatgtctaggtaaaggtggaatggattgttcgtttctaaagaagttgtttgggtcaaccaatgtcttaacctttactaatctatcgaaatttttaccgaagtatttttcaccccaaattctagcttgggtatagttgttaggattctttggatcgttaataccgatgtccaaatctctgtagttcaaatatgccaatctagggtttttagaaacgtatggagtcatgaagttatagatgtttctaatccagtttaagtgcttttcgttatcttcttgcttttcccatgaacaaatgtaccacaattcgtataagataccagctctatgaggaaatggaatggcagattcactgatttcgtccattataccaccgtatggatacaaggcgtacatgcctgcaccaatatcttcttcgtacaatttttctaagatttggacgaaaactgattcaggtattggctttttaacgtagtctaacttaattttaaaggcaccgttttgacctgcggatctatccaataatatttctttgttgaagttgtctgtatcgtagttgacaacacctgaataaaagatgatggtgtcgatccaagacaattgtctacaatcagttttcttaatacctaattctggaaaagacttattcatcaagtctactaaggaatcgacaccacccaagaaaactgaagaaaagtatgtgtggatagcagtcttatttttaccttggttatcggtgatgtttcttgtgatgaaatgagtcatcaacaacaagtccttatcgtacttgtatgcgatgttttgccacttattgaccaatttaactaattcatggatttccattatctttttgactgagaacatagtagactttggtactgcgactaatcttatcttccaagcaactatgataccgaatgattctgcaccaccacctctcaaagcccaaaataagtcttcacccatagactttctatccaaaactttaccgtgaacatttaccaaatgagcgtcgattatgttatcagcggccaaaccgtagtttctcattaaaggaccataaccaccaccaccaaaatgaccacctgcgcaaactgttggacagtaaccagcagccaatgataagttttcattcttttcgttaacccagtagtatacttcacccaatgttgcaccagcttcaacccaagcagtttgtgagtgtacgtctattttaattgatctcatgtttctcaaatcaacgataacgaatggaacttgggagatgtatgacatgccttcactatcatgaccaccggatctagttctaatttgcaaaccaacctttttagaacataagatagtaccttggatgtgagatacatgactaggggttacaatgaccaaaggttttggagtggtatcagaagtgaatctcaaattatggattgtactgttcaagacggacatgtacaatgggttgttttgagtgtaaaccaacttcaaattggtggcgttattaggtatgtattgtgagaagcacttcaaaaagttttctcttgggtttgcgatacttgtttggatgttaaaggaaaagaaaaagaagatgatcttgcatacgaaccaaaaggagaaagttgaacatttcataggacctggattttcttcaacgtcaccacaggtcaacaaagaacctctaccttcaataaaaacgtataccaaatattcagcgtagtacaatttccacataaactcgtagaatcttctacctgcttcagggtcataatttgtcaaagcgaaatctctagtttgcaagatcaaccagaaagccaagatggcatgtgacaacaacataacgttagaattaaaggcttgtggccaaatgatacctgccaaaatggctgcgacgtaacttaacaaaacgataccggagcagaacaaagtcaaatttcttgaaccgtacttagaagccaaggtactaataccgaactttgtgtcaccttcaacgtcagaggcatccttgatcaaggctaatgcagaacccatacttttcatgaatgccaacaaaaatgtgaatgaaggtctcaattcgaatggcaaacctaaagcagctcttgaagcgtagtagaaggtgaagtttgtgatgatatgagctaagaaattcaacaaaaaggcagtactagggttttgtttccatctaaaaggtggtacggaatagacaataccaccgaagataccgaaacagtaaccgaagatgtacaatggaccacccttcattttaattgtgatgatcaaaccgaacaaggctactatgatagacatgatccatgcagtattgacggatatttcacctgaagccaaaggcaaatctggtttgttaattctgtcgatgtgcaaatcgtatatttgattaattgtagtggtgaatgaagcgatgcacaagatggcaactaaaaagaaaaatgccttgaacatcaaggaccatgaaattaagttagtgttatgcaacaattctttaccgaataaaccgcatgcacaagaagtaaaagcgattatggtgtatggtctttgcaacttccaacatgctttaccgaagttcaaaatttttgtggcaacagagtgattatcactttcaggtggttcagtttgatttgtagttgcagctctgatagagttcttagctatagacaaactttcggagcacttattttgtaagtggaaggacttggttgaacaatgttttgatggaaagttgttgtaagagtacttaataggtgtctttggatgtctgtaacacaacaatgatgtttttggattgttgttgtgaggattcaataaggtatgatagttagtttggaaggagaaagtacagacggatgataaacccatactagttctagatccgtcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttaaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgccctgttctctgtagttgcgctaagagaatggacctatgaactgatggttggtgaagaaaacaatattttggtgctgggattctttttttttctggatgccagcttaaaaagcgggctccattatatttagtggatgccaggaataaacctgttcacccaagcaccatcagtgttatatattctgtgtaacccgccccctattttggcatgtacgggttacagcagaattaaaaggctaattttttgactaaataaagttaggaaaatcactactattaattatttacgtattctttgaaatggcagtattgataatgataaactcgagagctccagcttttgttcagttgattgtatgcttggtatagcttgaaatattgtgcagaaaaagaaacaaggaagaaagggaacgagaacaatgacgaggaaacaaaagattaataattgcaggtctatttatacttgatagcaagacagcaaacttttttttatttcaaattcaagtaactggaaggaaggccgtataccgttgctcattagagagtagtgtgcgtgaatgaaggaaggaaaaagtttcgtgtgcttcgagatacccctcatcagctctggaacaacgacatctgttggtgctgtctttgtcgttaattttttcctttagtgtcttccatcatttttttgtcattgcggatatggtgagacaacaacgggggagagagaaaagaaaaaaaaagaaaagaagttgcatgcgcctattattacttcaatagatggcaaatggaaaaagggtagtgaaacttcgatatgatgatggctatcaagtctagggctacagtattagttcgttatgtaccaccatcaatgaggcagtgtaattggtgtagtcttgtttagcccattatgtcttgtctggtatctgttctattgtatatctcccctccgccacctacatgttagggagaccaacgaaggtattataggaatcccgatgtatgggtttggttgccagaaaagaggaagtccatattgtacacccggaaacaacaaaaggatgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 5 - DNA, 8696 bp, synthetic construct
  misc_feature 1..8696: note = Codon Optimized
  source 1..8696: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaatgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgacttatgcatagtctggaacatcgtaagggtactttcttggggtgtaatcgaagatcaacaatttttcccagaaggatctgtaaacgtcaccaaaaccaacgtgagctggatgaatgatgtaatcttggatagtttcaactgattcgaaggttacttcgacaatgtgtgtataaccttcttctttcttttgtgtaacgtctttaccccagtatacatctttcatagcaggtataatgttgaccaaattaacgtaggtcttgaaaaattcttccttttgagcttctgtgatttcatctttaaacttcaatactatcaaatgcttgacggccataggacctgggttttcttcaacgtcaccacaagttaacaaggaacctctaccttcatatttaattggtactgatctgacaactactctttcgacggtcaaaccaggaccgaaaccaaataagacaccccattcaaaaccgtcaccagtagtagatttaccctcttctaatgatctctttctcaattcatccattacgaacaagacagtggatgaagacatgttaccgtgttcagataaaacatgtctactatctacaaacttttctttcttcaaatccaatttttcttcaaccttatccaaaatggctttaccacctggatgtgttatccagaaaatagagttccaatctgagatacctataggagtgaatgcttctatcaaacacttttctatgttgttagagattaacattggaacgtctttgtgcaaatcgaagatcaaacctgcttctcttatatgaccaccaattgtaccttcagaattaggcaagatggtttgacctgtactgactaattcaaatattggtctttcaccaacagattcgtcaggttctgcaccaacaataacagcagcagcaccgtcaccgaagatagcttgaccaactaacaattccaagtcagaatcacttggacctctaaacaagcaagccataatgtcgcaacaaacagctaatactctggcacccttgttgttttctgcaatatccttagcgattctcaaaacagtaccaccaccgtagcaacctaattgatacatcatgactctcttaacggatggtgacaaacctaacaatttggcacagtggtagtctgcaccaggcatatctgtagtagatgcacttgtaaaaatcaaatgagtgatctttgactttggttgaccccattccttaatggcttttgcacaagcatctttacccaatttaggaacttcgacaactaacatgtcttgtctggcatccaatgtttgcatttcgtgttctaccaatcttggattttgcttcaaatgttcttcgttcaagaagcagtttctctttctgatcatagacttatcacatatttttctaaacttttccttcaattgagtcatgtgttcactcttggtaactctgaagtaataatcaggaaattcatcttggatcaatatgttttctgggttggctgtacctatggctaatacggaggcaggaccttcggctctcaaatggttcatactagttctagatccgtcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttaaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgccctgttctctgtagttgcgctaagagaatggacctatgaactgatggttggtgaagaaaacaatattttggtgctgggattctttttttttctggatgccagcttaaaaagcgggctccattatatttagtggatgccaggaataaacctgttcacccaagcaccatcagtgttatatattctgtgtaacccgccccctattttggcatgtacgggttacagcagaattaaaaggctaattttttgactaaataaagttaggaaaatcactactattaattatttacgtattctttgaaatggcagtattgataatgataaactcgagagctccagcttttgttcagttgattgtatgcttggtatagcttgaaatattgtgcagaaaaagaaacaaggaagaaagggaacgagaacaatgacgaggaaacaaaagattaataattgcaggtctatttatacttgatagcaagacagcaaacttttttttatttcaaattcaagtaactggaaggaaggccgtataccgttgctcattagagagtagtgtgcgtgaatgaaggaaggaaaaagtttcgtgtgcttcgagatacccctcatcagctctggaacaacgacatctgttggtgctgtctttgtcgttaattttttcctttagtgtcttccatcatttttttgtcattgcggatatggtgagacaacaacgggggagagagaaaagaaaaaaaaagaaaagaagttgcatgcgcctattattacttcaatagatggcaaatggaaaaagggtagtgaaacttcgatatgatgatggctatcaagtctagggctacagtattagttcgttatgtaccaccatcaatgaggcagtgtaattggtgtagtcttgtttagcccattatgtcttgtctggtatctgttctattgtatatctcccctccgccacctacatgttagggagaccaacgaaggtattataggaatcccgatgtatgggtttggttgccagaaaagaggaagtccatattgtacacccggaaacaacaaaaggatgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 6 - DNA, 4322 bp, synthetic construct
  misc_feature 1..4322: note = Codon Optimized
  source 1..4322: mol_type = other DNA
aggaaacgaagataaatctcgagtttatcattatcaatactgccatttcaaagaatacgtaaataattaatagtagtgattttcctaactttatttagtcaaaaaattagccttttaattctgctgtaacccgtacatgccaaaatagggggcgggttacacagaatatataacactgatggtgcttgggtgaacaggtttattcctggcatccactaaatataatggagcccgctttttaagctggcatccagaaaaaaaaagaatcccagcaccaaaatattgttttcttcaccaaccatcagttcataggtccattctcttagcgcaactacagagaacagggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggtttaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatctagaactagtatgggtaaaaactataagtccttggattcagtcgttgcctcagatttcatcgcattgggtatcacctcagaagtagcagaaacattacatggtagattggcagaaatcgtttgtaattatggtgctgcaacccctcaaacttggatcaacatcgctaaccatatcttgtcaccagatttgcctttctccttacaccaaatgttgttttatggttgctacaaggatttcggtccagccccacctgcttggattccagaccctgaaaaagtcaagtcaactaatttgggtgctttgttggaaaagagaggtaaagaatttttgggtgtaaagtacaaagatccaatttcttctttttctcacttccaagaattttctgttagaaaccctgaagtctattggagaacagtattgatggatgaaatgaaaattagtttctctaaggacccagaatgtatcttgagaagagatgacatcaacaacccaggtggttctgaatggttacctggtggttacttgaactcagctaaaaattgcttgaacgtaaactccaataagaaattgaacgatactatgatcgtttggagagacgagggtaacgatgacttgcctttgaataagttgacattagatcaattgagaaagagagtttggttggttggttatgcattggaagaaatgggtttagaaaaaggttgtgcaatagccatcgatatgccaatgcatgttgatgctgttgttatatatttggccatagtattggctggttacgtagttgtctctatagcagattcattttccgcccctgaaatctcaactagattgagattatccaaagctaaggcaattttcacacaagatcacatcatcagaggtaaaaagagaataccattgtattcaagagtagttgaagctaaatccccaatggcaatagttatcccttgtagtggttctaacattggtgcagaattgagagatggtgacatatcttgggattactttttagaaagagccaaggagtttaaaaactgcgagtttactgccagagaacaacctgttgatgcttatactaacatcttattctccagtggtactacaggtgaaccaaaagcaattccttggacacaagccaccccattgaaggctgctgctgatggttggtctcatttggatattagaaaaggtgacgttatagtatggccaactaatttgggttggatgatgggtccttggttggtttatgctagtttgttaaatggtgcatctattgccttgtacaacggtagtcctttagtctctggtttcgctaaatttgttcaagatgcaaaggtcacaatgttgggtgtcgtaccatctattgtaagatcatggaaatccacaaattgtgtttcaggttacgattggtccaccataagatgcttttcttcatccggtgaagcctctaatgtagacgaatatttgtggttaatgggtagagctaactacaagccagttatagaaatgtgtggtggtacagaaatcggtggtgctttttctgctggttcatttttgcaagctcaatctttaagttctttttcatcccaatgtatgggttgcaccttgtacatattagataagaacggttacccaatgcctaaaaataagccaggtatcggtgaattggcattaggtcctgttatgtttggtgcctcaaaaacattgttaaacggtaatcatcacgatgtctatttcaagggtatgccaaccttgaatggtgaagtattgagaagacatggtgacattttcgaattgacctctaacggttactaccatgcacacggtagagccgatgacactatgaacatcggtggtatcaaaattagttctatcgaaatcgaaagagtctgtaatgaagtagatgacagagtttttgaaaccactgctattggtgttccacctttgggtggtggtccagaacaattggtcatatttttcgtattgaaggattcaaacgacacaaccattgatttgaaccaattgagattatcctttaacttgggtttgcaaaagaaattgaacccattattcaaagttactagagttgtcccattgtcatccttacctagaactgcaacaaacaagatcatgagaagagttttgagacaacaattcagtcatttcgaagaaggtagaggttctttgttaacatgtggtgacgttgaagaaaatccaggtcctatggcttcagaaaaggaaataagaagagaaagattcttgaacgtattcccaaagttagttgaagaattgaacgctagtttgttagcttatggtatgcctaaagaagcctgcgattggtatgctcactctttaaactacaatactccaggtggtaaattgaatagaggtttgagtgtagttgatacttatgctatcttgtctaacaaaaccgttgaacaattaggtcaagaagaatacgaaaaggtcgctatcttgggttggtgtattgaattgttgcaagcatactttttggttgccgatgacatgatggataagtctataacaagaagaggtcaaccatgctggtacaaagttccagaagttggtgaaatagccataaatgatgcttttatgttggaagccgctatctataaattgttgaagtcacatttcagaaacgaaaagtactacatcgatattaccgaattattccacgaagttactttccaaacagaattgggtcaattgatggatttgataactgcacctgaagataaagttgacttgtcaaagttttccttgaagaaacattcattcatcgtcacctttgaaactgcttattactccttctatttgccagtcgccttggctatgtacgtagctggtattactgatgaaaaagacttgaagcaagcaagagatgttttgatacctttgggtgaatacttccaaatccaagatgactacttagactgtttcggtactccagaacaaataggtaaaatcggtacagatattcaagacaataagtgcagttgggttattaacaaggctttggaattagcatctgccgaacaaagaaagactttggatgaaaactacggtaaaaaggactcagttgctgaagcaaagtgtaagaaaatttttaatgatttgaagattgaacaattgtaccatgaatacgaagaatccatcgctaaagacttaaaggcaaagattagtcaagttgatgaatcaagaggttttaaagccgacgttttgacagctttcttgaataaggtctacaagagatcaaaggattacaaggatcatgacggtgactataaagaccacgatattgactacaaagatgacgatgacaagtaagtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgtgacataactaattacatgacttgactgatttttcc

SEQ ID NO: 7 - DNA, 2522 bp, synthetic construct
  misc_feature 1..2522: note = Codon Optimized
  source 1..2522: mol_type = other DNA
aggaaacgaagataaatctcgagtttatcattatcaatactgccatttcaaagaatacgtaaataattaatagtagtgattttcctaactttatttagtcaaaaaattagccttttaattctgctgtaacccgtacatgccaaaatagggggcgggttacacagaatatataacactgatggtgcttgggtgaacaggtttattcctggcatccactaaatataatggagcccgctttttaagctggcatccagaaaaaaaaagaatcccagcaccaaaatattgttttcttcaccaaccatcagttcataggtccattctcttagcgcaactacagagaacagggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggtttaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatctagaactagtatgaaccatttgagagccgaaggtcctgcctccgtattagccataggtacagccaacccagaaaacatattgatccaagatgaatttcctgattattacttcagagttaccaagagtgaacacatgactcaattgaaggaaaagtttagaaaaatatgtgataagtctatgatcagaaagagaaactgcttcttgaacgaagaacatttgaagcaaaatccaagattggtagaacacgaaatgcaaacattggatgccagacaagacatgttagttgtcgaagttcctaaattgggtaaagatgcttgtgcaaaagccattaaggaatggggtcaaccaaagtcaaagatcactcatttgatttttacaagtgcatctactacagatatgcctggtgcagactaccactgtgccaaattgttaggtttgtcaccatccgttaagagagtcatgatgtatcaattaggttgctacggtggtggtactgttttgagaatcgctaaggatattgcagaaaacaacaagggtgccagagtattagctgtttgttgcgacattatggcttgcttgtttagaggtccaagtgattctgacttggaattgttagttggtcaagctatcttcggtgacggtgctgctgctgttattgttggtgcagaacctgacgaatctgttggtgaaagaccaatatttgaattagtcagtacaggtcaaaccatcttgcctaattctgaaggtacaattggtggtcatataagagaagcaggtttgatcttcgatttgcacaaagacgttccaatgttaatctctaacaacatagaaaagtgtttgatagaagcattcactcctataggtatctcagattggaactctattttctggataacacatccaggtggtaaagccattttggataaggttgaagaaaaattggatttgaagaaagaaaagtttgtagatagtagacatgttttatctgaacacggtaacatgtcttcatccactgtcttgttcgtaatggatgaattgagaaagagatcattagaagagggtaaatctactactggtgacggttttgaatggggtgtcttatttggtttcggtcctggtttgaccgtcgaaagagtagttgtcagatcagtaccaattaaatatgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtacccttacgatgttccagactatgcataagtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgtgacataactaattacatgacttgactgatttttcc

SEQ ID NO: 8 - DNA, 3887 bp, synthetic construct
  misc_feature 1..3887: note = Codon Optimized
  source 1..3887: mol_type = other DNA
aggaaacgaagataaatctcgagtttatcattatcaatactgccatttcaaagaatacgtaaataattaatagtagtgattttcctaactttatttagtcaaaaaattagccttttaattctgctgtaacccgtacatgccaaaatagggggcgggttacacagaatatataacactgatggtgcttgggtgaacaggtttattcctggcatccactaaatataatggagcccgctttttaagctggcatccagaaaaaaaaagaatcccagcaccaaaatattgttttcttcaccaaccatcagttcataggtccattctcttagcgcaactacagagaacagggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggtttaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatctagaactagtatgggtttatcatccgtctgtactttctccttccaaactaactatcataccttattgaatcctcacaacaacaatccaaaaacatcattgttgtgttacagacatccaaagacacctattaagtactcttacaacaactttccatcaaaacattgttcaaccaagtccttccacttacaaaataagtgctccgaaagtttgtctatagctaagaactctatcagagctgcaactacaaatcaaactgaaccacctgaaagtgataatcactctgttgccacaaaaattttgaacttcggtaaagcatgttggaagttgcaaagaccatacaccataatcgcttttacttcttgtgcatgcggtttattcggtaaagaattgttgcataacactaacttaatttcatggtccttgatgttcaaggcatttttctttttagttgccatcttgtgcatcgcttcattcaccactacaattaatcaaatatacgatttgcacatcgacagaattaacaaaccagatttgcctttggcttcaggtgaaatatccgtcaatactgcatggatcatgtctatcatagtagccttgttcggtttgatcatcacaattaaaatgaagggtggtccattgtacatcttcggttactgtttcggtatcttcggtggtattgtctattccgtaccaccttttagatggaaacaaaaccctagtactgcctttttgttgaatttcttagctcatatcatcacaaacttcaccttctactacgcttcaagagctgctttaggtttgccattcgaattgagaccttcattcacatttttgttggcattcatgaaaagtatgggttctgcattagccttgatcaaggatgcctctgacgttgaaggtgacacaaagttcggtattagtaccttggcttctaagtacggttcaagaaatttgactttgttctgctccggtatcgttttgttaagttacgtcgcagccattttggcaggtatcatttggccacaagcctttaattctaacgttatgttgttgtcacatgccatcttggctttctggttgatcttgcaaactagagatttcgctttgacaaattatgaccctgaagcaggtagaagattctacgagtttatgtggaaattgtactacgctgaatatttggtatacgtttttattgaaggtagaggttctttgttgacctgtggtgacgttgaagaaaatccaggtcctatgaaatgttcaactttctccttttggttcgtatgcaagatcatcttctttttcttttcctttaacatccaaacaagtatcgcaaacccaagagaaaactttttgaagtgcttctcacaatacatacctaataacgccaccaatttgaagttggtttacactcaaaacaacccattgtacatgtccgtcttgaacagtacaatccataatttgagattcacttctgataccactccaaaacctttggtcattgtaacccctagtcatgtatctcacatccaaggtactatcttatgttctaaaaaggttggtttgcaaattagaactagatccggtggtcatgatagtgaaggcatgtcatacatctcccaagttccattcgttatcgttgatttgagaaacatgagatcaattaaaatagacgtacactcacaaactgcttgggttgaagctggtgcaacattgggtgaagtatactactgggttaacgaaaagaatgaaaacttatcattggctgctggttactgtccaacagtttgcgcaggtggtcattttggtggtggtggttatggtcctttaatgagaaactacggtttggccgctgataacataatcgacgctcatttggtaaatgttcacggtaaagttttggatagaaagtctatgggtgaagacttattttgggctttgagaggtggtggtgcagaatcattcggtatcatagttgcttggaagataagattagtcgcagtaccaaagtctactatgttctcagtcaaaaagataatggaaatccatgaattagttaaattggtcaataagtggcaaaacatcgcatacaagtacgataaggacttgttgttgatgactcatttcatcacaagaaacatcaccgataaccaaggtaaaaataagactgctatccacacatacttttcttcagttttcttgggtggtgtcgattccttagtagacttgatgaataagtcttttccagaattaggtattaagaaaactgattgtagacaattgtcttggatcgacaccatcatcttttattcaggtgttgtcaactacgatacagacaacttcaacaaagaaatattattggatagatccgcaggtcaaaacggtgcctttaaaattaagttagactacgttaaaaagccaatacctgaatcagttttcgtccaaatcttagaaaaattgtacgaagaagatattggtgcaggcatgtacgccttgtatccatacggtggtataatggacgaaatcagtgaatctgccattccatttcctcatagagctggtatcttatacgaattgtggtacatttgttcatgggaaaagcaagaagataacgaaaagcacttaaactggattagaaacatctataacttcatgactccatacgtttctaaaaaccctagattggcatatttgaactacagagatttggacatcggtattaacgatccaaagaatcctaacaactatacccaagctagaatttggggtgaaaaatacttcggtaaaaatttcgatagattagtaaaggttaagacattggttgacccaaacaacttctttagaaacgaacaatccattccacctttacctagacatagacacgaacaaaaattaataagtgaagaagatttgtaagtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgtgacataactaattacatgacttgactgatttttcc

SEQ ID NO: 9 - DNA, 8963 bp, synthetic construct
  misc_feature 1..8963: note = Codon Optimized
  source 1..8963: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgacttacttgtcatcgtcatctttgtagtcaatatcgtggtctttatagtcaccgtcatgatccttgtaatcctttgatctcttgtagaccttattcaagaaagctgtcaaaacgtcggctttaaaacctcttgattcatcaacttgactaatctttgcctttaagtctttagcgatggattcttcgtattcatggtacaattgttcaatcttcaaatcattaaaaattttcttacactttgcttcagcaactgagtcctttttaccgtagttttcatccaaagtctttctttgttcggcagatgctaattccaaagccttgttaataacccaactgcacttattgtcttgaatatctgtaccgattttacctatttgttctggagtaccgaaacagtctaagtagtcatcttggatttggaagtattcacccaaaggtatcaaaacatctcttgcttgcttcaagtctttttcatcagtaataccagctacgtacatagccaaggcgactggcaaatagaaggagtaataagcagtttcaaaggtgacgatgaatgaatgtttcttcaaggaaaactttgacaagtcaactttatcttcaggtgcagttatcaaatccatcaattgacccaattctgtttggaaagtaacttcgtggaataattcggtaatatcgatgtagtacttttcgtttctgaaatgtgacttcaacaatttatagatagcggcttccaacataaaagcatcatttatggctatttcaccaacttctggaactttgtaccagcatggttgacctcttcttgttatagacttatccatcatgtcatcggcaaccaaaaagtatgcttgcaacaattcaatacaccaacccaagatagcgaccttttcgtattcttcttgacctaattgttcaacggttttgttagacaagatagcataagtatcaactacactcaaacctctattcaatttaccacctggagtattgtagtttaaagagtgagcataccaatcgcaggcttctttaggcataccataagctaacaaactagcgttcaattcttcaactaactttgggaatacgttcaagaatctttctcttcttatttccttttctgaagccataggacctggattttcttcaacgtcaccacatgttaacaaagaacctctaccttcttcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccatactagttctagatccgtcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttaaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgccctgttctctgtagttgcgctaagagaatggacctatgaactgatggttggtgaagaaaacaatattttggtgctgggattctttttttttctggatgccagcttaaaaagcgggctccattatatttagtggatgccaggaataaacctgttcacccaagcaccatcagtgttatatattctgtgtaacccgccccctattttggcatgtacgggttacagcagaattaaaaggctaattttttgactaaataaagttaggaaaatcactactattaattatttacgtattctttgaaatggcagtattgataatgataaactcgagagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 10 - DNA, 7163 bp, synthetic construct
  misc_feature 1..7163: note = Codon Optimized
  source 1..7163: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgacttatgcatagtctggaacatcgtaagggtactttcttggggtgtaatcgaagatcaacaatttttcccagaaggatctgtaaacgtcaccaaaaccaacgtgagctggatgaatgatgtaatcttggatagtttcaactgattcgaaggttacttcgacaatgtgtgtataaccttcttctttcttttgtgtaacgtctttaccccagtatacatctttcatagcaggtataatgttgaccaaattaacgtaggtcttgaaaaattcttccttttgagcttctgtgatttcatctttaaacttcaatactatcaaatgcttgacggccataggacctgggttttcttcaacgtcaccacaagttaacaaggaacctctaccttcatatttaattggtactgatctgacaactactctttcgacggtcaaaccaggaccgaaaccaaataagacaccccattcaaaaccgtcaccagtagtagatttaccctcttctaatgatctctttctcaattcatccattacgaacaagacagtggatgaagacatgttaccgtgttcagataaaacatgtctactatctacaaacttttctttcttcaaatccaatttttcttcaaccttatccaaaatggctttaccacctggatgtgttatccagaaaatagagttccaatctgagatacctataggagtgaatgcttctatcaaacacttttctatgttgttagagattaacattggaacgtctttgtgcaaatcgaagatcaaacctgcttctcttatatgaccaccaattgtaccttcagaattaggcaagatggtttgacctgtactgactaattcaaatattggtctttcaccaacagattcgtcaggttctgcaccaacaataacagcagcagcaccgtcaccgaagatagcttgaccaactaacaattccaagtcagaatcacttggacctctaaacaagcaagccataatgtcgcaacaaacagctaatactctggcacccttgttgttttctgcaatatccttagcgattctcaaaacagtaccaccaccgtagcaacctaattgatacatcatgactctcttaacggatggtgacaaacctaacaatttggcacagtggtagtctgcaccaggcatatctgtagtagatgcacttgtaaaaatcaaatgagtgatctttgactttggttgaccccattccttaatggcttttgcacaagcatctttacccaatttaggaacttcgacaactaacatgtcttgtctggcatccaatgtttgcatttcgtgttctaccaatcttggattttgcttcaaatgttcttcgttcaagaagcagtttctctttctgatcatagacttatcacatatttttctaaacttttccttcaattgagtcatgtgttcactcttggtaactctgaagtaataatcaggaaattcatcttggatcaatatgttttctgggttggctgtacctatggctaatacggaggcaggaccttcggctctcaaatggttcatactagttctagatccgtcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttaaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgccctgttctctgtagttgcgctaagagaatggacctatgaactgatggttggtgaagaaaacaatattttggtgctgggattctttttttttctggatgccagcttaaaaagcgggctccattatatttagtggatgccaggaataaacctgttcacccaagcaccatcagtgttatatattctgtgtaacccgccccctattttggcatgtacgggttacagcagaattaaaaggctaattttttgactaaataaagttaggaaaatcactactattaattatttacgtattctttgaaatggcagtattgataatgataaactcgagagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 11 - DNA, 8710 bp, synthetic construct
  misc_feature 1..8710: note = Codon Optimized
  source 1..8710: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgacttacaaatcttcttcacttattaatttttgttcgtgtctatgtctaggtaaaggtggaatggattgttcgtttctaaagaagttgtttgggtcaaccaatgtcttaacctttactaatctatcgaaatttttaccgaagtatttttcaccccaaattctagcttgggtatagttgttaggattctttggatcgttaataccgatgtccaaatctctgtagttcaaatatgccaatctagggtttttagaaacgtatggagtcatgaagttatagatgtttctaatccagtttaagtgcttttcgttatcttcttgcttttcccatgaacaaatgtaccacaattcgtataagataccagctctatgaggaaatggaatggcagattcactgatttcgtccattataccaccgtatggatacaaggcgtacatgcctgcaccaatatcttcttcgtacaatttttctaagatttggacgaaaactgattcaggtattggctttttaacgtagtctaacttaattttaaaggcaccgttttgacctgcggatctatccaataatatttctttgttgaagttgtctgtatcgtagttgacaacacctgaataaaagatgatggtgtcgatccaagacaattgtctacaatcagttttcttaatacctaattctggaaaagacttattcatcaagtctactaaggaatcgacaccacccaagaaaactgaagaaaagtatgtgtggatagcagtcttatttttaccttggttatcggtgatgtttcttgtgatgaaatgagtcatcaacaacaagtccttatcgtacttgtatgcgatgttttgccacttattgaccaatttaactaattcatggatttccattatctttttgactgagaacatagtagactttggtactgcgactaatcttatcttccaagcaactatgataccgaatgattctgcaccaccacctctcaaagcccaaaataagtcttcacccatagactttctatccaaaactttaccgtgaacatttaccaaatgagcgtcgattatgttatcagcggccaaaccgtagtttctcattaaaggaccataaccaccaccaccaaaatgaccacctgcgcaaactgttggacagtaaccagcagccaatgataagttttcattcttttcgttaacccagtagtatacttcacccaatgttgcaccagcttcaacccaagcagtttgtgagtgtacgtctattttaattgatctcatgtttctcaaatcaacgataacgaatggaacttgggagatgtatgacatgccttcactatcatgaccaccggatctagttctaatttgcaaaccaacctttttagaacataagatagtaccttggatgtgagatacatgactaggggttacaatgaccaaaggttttggagtggtatcagaagtgaatctcaaattatggattgtactgttcaagacggacatgtacaatgggttgttttgagtgtaaaccaacttcaaattggtggcgttattaggtatgtattgtgagaagcacttcaaaaagttttctcttgggtttgcgatacttgtttggatgttaaaggaaaagaaaaagaagatgatcttgcatacgaaccaaaaggagaaagttgaacatttcataggacctggattttcttcaacgtcaccacaggtcaacaaagaacctctaccttcaataaaaacgtataccaaatattcagcgtagtacaatttccacataaactcgtagaatcttctacctgcttcagggtcataatttgtcaaagcgaaatctctagtttgcaagatcaaccagaaagccaagatggcatgtgacaacaacataacgttagaattaaaggcttgtggccaaatgatacctgccaaaatggctgcgacgtaacttaacaaaacgataccggagcagaacaaagtcaaatttcttgaaccgtacttagaagccaaggtactaataccgaactttgtgtcaccttcaacgtcagaggcatccttgatcaaggctaatgcagaacccatacttttcatgaatgccaacaaaaatgtgaatgaaggtctcaattcgaatggcaaacctaaagcagctcttgaagcgtagtagaaggtgaagtttgtgatgatatgagctaagaaattcaacaaaaaggcagtactagggttttgtttccatctaaaaggtggtacggaatagacaataccaccgaagataccgaaacagtaaccgaagatgtacaatggaccacccttcattttaattgtgatgatcaaaccgaacaaggctactatgatagacatgatccatgcagtattgacggatatttcacctgaagccaaaggcaaatctggtttgttaattctgtcgatgtgcaaatcgtatatttgattaattgtagtggtgaatgaagcgatgcacaagatggcaactaaaaagaaaaatgccttgaacatcaaggaccatgaaattaagttagtgttatgcaacaattctttaccgaataaaccgcatgcacaagaagtaaaagcgattatggtgtatggtctttgcaacttccaacatgctttaccgaagttcaaaatttttgtggcaacagagtgattatcactttcaggtggttcagtttgatttgtagttgcagctctgatagagttcttagctatagacaaactttcggagcacttattttgtaagtggaaggacttggttgaacaatgttttgatggaaagttgttgtaagagtacttaataggtgtctttggatgtctgtaacacaacaatgatgtttttggattgttgttgtgaggattcaataaggtatgatagttagtttggaaggagaaagtacagacggatgataaacccatactagttctagatccgtcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttaaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgccctgttctctgtagttgcgctaagagaatggacctatgaactgatggttggtgaagaaaacaatattttggtgctgggattctttttttttctggatgccagcttaaaaagcgggctccattatatttagtggatgccaggaataaacctgttcacccaagcaccatcagtgttatatattctgtgtaacccgccccctattttggcatgtacgggttacagcagaattaaaaggctaattttttgactaaataaagttaggaaaatcactactattaattatttacgtattctttgaaatggcagtattgataatgataaactcgagagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 12 - DNA, 9617 bp, synthetic construct
  misc_feature 1..9617: note = Codon Optimized
  source 1..9617: mol_type = other DNA
ttatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatgaaccatttgagagccgaaggtcctgcctccgtattagccataggtacagccaacccagaaaacatattgatccaagatgaatttcctgattattacttcagagttaccaagagtgaacacatgactcaattgaaggaaaagtttagaaaaatatgtgataagtctatgatcagaaagagaaactgcttcttgaacgaagaacatttgaagcaaaatccaagattggtagaacacgaaatgcaaacattggatgccagacaagacatgttagttgtcgaagttcctaaattgggtaaagatgcttgtgcaaaagccattaaggaatggggtcaaccaaagtcaaagatcactcatttgatttttacaagtgcatctactacagatatgcctggtgcagactaccactgtgccaaattgttaggtttgtcaccatccgttaagagagtcatgatgtatcaattaggttgctacggtggtggtactgttttgagaatcgctaaggatattgcagaaaacaacaagggtgccagagtattagctgtttgttgcgacattatggcttgcttgtttagaggtccaagtgattctgacttggaattgttagttggtcaagctatcttcggtgacggtgctgctgctgttattgttggtgcagaacctgacgaatctgttggtgaaagaccaatatttgaattagtcagtacaggtcaaaccatcttgcctaattctgaaggtacaattggtggtcatataagagaagcaggtttgatcttcgatttgcacaaagacgttccaatgttaatctctaacaacatagaaaagtgtttgatagaagcattcactcctataggtatctcagattggaactctattttctggataacacatccaggtggtaaagccattttggataaggttgaagaaaaattggatttgaagaaagaaaagtttgtagatagtagacatgttttatctgaacacggtaacatgtcttcatccactgtcttgttcgtaatggatgaattgagaaagagatcattagaagagggtaaatctactactggtgacggttttgaatggggtgtcttatttggtttcggtcctggtttgaccgtcgaaagagtagttgtcagatcagtaccaattaaatatgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagttaaagccaaaataatgataacgagaataatatcaagaataccttagaacaacatcgacaacaacaacaggcattttcggatatgagtcacgtggagtattccagaattacaaaattttttcaagaacaaccactggagggatatacccttttctctcacaggtctgcgccatgggtttatcatccgtctgtactttctccttccaaactaactatcataccttattgaatcctcacaacaacaatccaaaaacatcattgttgtgttacagacatccaaagacacctattaagtactcttacaacaactttccatcaaaacattgttcaaccaagtccttccacttacaaaataagtgctccgaaagtttgtctatagctaagaactctatcagagctgcaactacaaatcaaactgaaccacctgaaagtgataatcactctgttgccacaaaaattttgaacttcggtaaagcatgttggaagttgcaaagaccatacaccataatcgcttttacttcttgtgcatgcggtttattcggtaaagaattgttgcataacactaacttaatttcatggtccttgatgttcaaggcatttttctttttagttgccatcttgtgcatcgcttcattcaccactacaattaatcaaatatacgatttgcacatcgacagaattaacaaaccagatttgcctttggcttcaggtgaaatatccgtcaatactgcatggatcatgtctatcatagtagccttgttcggtttgatcatcacaattaaaatgaagggtggtccattgtacatcttcggttactgtttcggtatcttcggtggtattgtctattccgtaccaccttttagatggaaacaaaaccctagtactgcctttttgttgaatttcttagctcatatcatcacaaacttcaccttctactacgcttcaagagctgctttaggtttgccattcgaattgagaccttcattcacatttttgttggcattcatgaaaagtatgggttctgcattagccttgatcaaggatgcctctgacgttgaaggtgacacaaagttcggtattagtaccttggcttctaagtacggttcaagaaatttgactttgttctgctccggtatcgttttgttaagttacgtcgcagccattttggcaggtatcatttggccacaagcctttaattctaacgttatgttgttgtcacatgccatcttggctttctggttgatcttgcaaactagagatttcgctttgacaaattatgaccctgaagcaggtagaagattctacgagtttatgtggaaattgtactacgctgaatatttggtatacgtttttattgaaggtagaggttctttgttgacctgtggtgacgttgaagaaaatccaggtcctatgaaatgttcaactttctccttttggttcgtatgcaagatcatcttctttttcttttcctttaacatccaaacaagtatcgcaaacccaagagaaaactttttgaagtgcttctcacaatacatacctaataacgccaccaatttgaagttggtttacactcaaaacaacccattgtacatgtccgtcttgaacagtacaatccataatttgagattcacttctgataccactccaaaacctttggtcattgtaacccctagtcatgtatctcacatccaaggtactatcttatgttctaaaaaggttggtttgcaaattagaactagatccggtggtcatgatagtgaaggcatgtcatacatctcccaagttccattcgttatcgttgatttgagaaacatgagatcaattaaaatagacgtacactcacaaactgcttgggttgaagctggtgcaacattgggtgaagtatactactgggttaacgaaaagaatgaaaacttatcattggctgctggttactgtccaacagtttgcgcaggtggtcattttggtggtggtggttatggtcctttaatgagaaactacggtttggccgctgataacataatcgacgctcatttggtaaatgttcacggtaaagttttggatagaaagtctatgggtgaagacttattttgggctttgagaggtggtggtgcagaatcattcggtatcatagttgcttggaagataagattagtcgcagtaccaaagtctactatgttctcagtcaaaaagataatggaaatccatgaattagttaaattggtcaataagtggcaaaacatcgcatacaagtacgataaggacttgttgttgatgactcatttcatcacaagaaacatcaccgataaccaaggtaaaaataagactgctatccacacatacttttcttcagttttcttgggtggtgtcgattccttagtagacttgatgaataagtcttttccagaattaggtattaagaaaactgattgtagacaattgtcttggatcgacaccatcatcttttattcaggtgttgtcaactacgatacagacaacttcaacaaagaaatattattggatagatccgcaggtcaaaacggtgcctttaaaattaagttagactacgttaaaaagccaatacctgaatcagttttcgtccaaatcttagaaaaattgtacgaagaagatattggtgcaggcatgtacgccttgtatccatacggtggtataatggacgaaatcagtgaatctgccattccatttcctcatagagctggtatcttatacgaattgtggtacatttgttcatgggaaaagcaagaagataacgaaaagcacttaaactggattagaaacatctataacttcatgactccatacgtttctaaaaaccctagattggcatatttgaactacagagatttggacatcggtattaacgatccaaagaatcctaacaactatacccaagctagaatttggggtgaaaaatacttcggtaaaaatttcgatagattagtaaaggttaagacattggttgacccaaacaacttctttagaaacgaacaatccattccacctttacctagacatagacactgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccatta

SEQ ID NO: 13 - DNA, 7879 bp, synthetic construct
  misc_feature 1..7879: note = Codon Optimized
  source 1..7879: mol_type = other DNA
aacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcatgaaccatttgagagccgaaggtcctgcctccgtattagccataggtacagccaacccagaaaacatattgatccaagatgaatttcctgattattacttcagagttaccaagagtgaacacatgactcaattgaaggaaaagtttagaaaaatatgtgataagtctatgatcagaaagagaaactgcttcttgaacgaagaacatttgaagcaaaatccaagattggtagaacacgaaatgcaaacattggatgccagacaagacatgttagttgtcgaagttcctaaattgggtaaagatgcttgtgcaaaagccattaaggaatggggtcaaccaaagtcaaagatcactcatttgatttttacaagtgcatctactacagatatgcctggtgcagactaccactgtgccaaattgttaggtttgtcaccatccgttaagagagtcatgatgtatcaattaggttgctacggtggtggtactgttttgagaatcgctaaggatattgcagaaaacaacaagggtgccagagtattagctgtttgttgcgacattatggcttgcttgtttagaggtccaagtgattctgacttggaattgttagttggtcaagctatcttcggtgacggtgctgctgctgttattgttggtgcagaacctgacgaatctgttggtgaaagaccaatatttgaattagtcagtacaggtcaaaccatcttgcctaattctgaaggtacaattggtggtcatataagagaagcaggtttgatcttcgatttgcacaaagacgttccaatgttaatctctaacaacatagaaaagtgtttgatagaagcattcactcctataggtatctcagattggaactctattttctggataacacatccaggtggtaaagccattttggataaggttgaagaaaaattggatttgaagaaagaaaagtttgtagatagtagacatgttttatctgaacacggtaacatgtcttcatccactgtcttgttcgtaatggatgaattgagaaagagatcattagaagagggtaaatctactactggtgacggttttgaatggggtgtcttatttggtttcggtcctggtttgaccgtcgaaagagtagttgtcagatcagtaccaattaaatatgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgataacgagaataatatcaagaataccttagaacaacatcgacaacaacaacaggcattttcggatatgagtcacgtggagtattccagaattacaaaattttttcaagaacaaccactggagggatatacccttttctctcacaggtctgcgccatgggtttatcatccgtctgtactttctccttccaaactaactatcataccttattgaatcctcacaacaacaatccaaaaacatcattgttgtgttacagacatccaaagacacctattaagtactcttacaacaactttccatcaaaacattgttcaaccaagtccttccacttacaaaataagtgctccgaaagtttgtctatagctaagaactctatcagagctgcaactacaaatcaaactgaaccacctgaaagtgataatcactctgttgccacaaaaattttgaacttcggtaaagcatgttggaagttgcaaagaccatacaccataatcgcttttacttcttgtgcatgcggtttattcggtaaagaattgttgcataacactaacttaatttcatggtccttgatgttcaaggcatttttctttttagttgccatcttgtgcatcgcttcattcaccactacaattaatcaaatatacgatttgcacatcgacagaattaacaaaccagatttgcctttggcttcaggtgaaatatccgtcaatactgcatggatcatgtctatcatagtagccttgttcggtttgatcatcacaattaaaatgaagggtggtccattgtacatcttcggttactgtttcggtatcttcggtggtattgtctattccgtaccaccttttagatggaaacaaaaccctagtactgcctttttgttgaatttcttagctcatatcatcacaaacttcaccttctactacgcttcaagagctgctttaggtttgccattcgaattgagaccttcattcacatttttgttggcattcatgaaaagtatgggttctgcattagccttgatcaaggatgcctctgacgttgaaggtgacacaaagttcggtattagtaccttggcttctaagtacggttcaagaaatttgactttgttctgctccggtatcgttttgttaagttacgtcgcagccattttggcaggtatcatttggccacaagcctttaattctaacgttatgttgttgtcacatgccatcttggctttctggttgatcttgcaaactagagatttcgctttgacaaattatgaccctgaagcaggtagaagattctacgagtttatgtggaaattgtactacgctgaatatttggtatacgtttttatttaacgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacatt

SEQ ID NO: 14 - DNA, 3353 bp, synthetic construct
  misc_feature 1..3353: note = Codon Optimized
  source 1..3353: mol_type = other DNA
actagtatgggtaaaaactataagtccttggattcagtcgttgcctcagatttcatcgcattgggtatcacctcagaagtagcagaaacattacatggtagattggcagaaatcgtttgtaattatggtgctgcaacccctcaaacttggatcaacatcgctaaccatatcttgtcaccagatttgcctttctccttacaccaaatgttgttttatggttgctacaaggatttcggtccagccccacctgcttggattccagaccctgaaaaagtcaagtcaactaatttgggtgctttgttggaaaagagaggtaaagaatttttgggtgtaaagtacaaagatccaatttcttctttttctcacttccaagaattttctgttagaaaccctgaagtctattggagaacagtattgatggatgaaatgaaaattagtttctctaaggacccagaatgtatcttgagaagagatgacatcaacaacccaggtggttctgaatggttacctggtggttacttgaactcagctaaaaattgcttgaacgtaaactccaataagaaattgaacgatactatgatcgtttggagagacgagggtaacgatgacttgcctttgaataagttgacattagatcaattgagaaagagagtttggttggttggttatgcattggaagaaatgggtttagaaaaaggttgtgcaatagccatcgatatgccaatgcatgttgatgctgttgttatatatttggccatagtattggctggttacgtagttgtctctatagcagattcattttccgcccctgaaatctcaactagattgagattatccaaagctaaggcaattttcacacaagatcacatcatcagaggtaaaaagagaataccattgtattcaagagtagttgaagctaaatccccaatggcaatagttatcccttgtagtggttctaacattggtgcagaattgagagatggtgacatatcttgggattactttttagaaagagccaaggagtttaaaaactgcgagtttactgccagagaacaacctgttgatgcttatactaacatcttattctccagtggtactacaggtgaaccaaaagcaattccttggacacaagccaccccattgaaggctgctgctgatggttggtctcatttggatattagaaaaggtgacgttatagtatggccaactaatttgggttggatgatgggtccttggttggtttatgctagtttgttaaatggtgcatctattgccttgtacaacggtagtcctttagtctctggtttcgctaaatttgttcaagatgcaaaggtcacaatgttgggtgtcgtaccatctattgtaagatcatggaaatccacaaattgtgtttcaggttacgattggtccaccataagatgcttttcttcatccggtgaagcctctaatgtagacgaatatttgtggttaatgggtagagctaactacaagccagttatagaaatgtgtggtggtacagaaatcggtggtgctttttctgctggttcatttttgcaagctcaatctttaagttctttttcatcccaatgtatgggttgcaccttgtacatattagataagaacggttacccaatgcctaaaaataagccaggtatcggtgaattggcattaggtcctgttatgtttggtgcctcaaaaacattgttaaacggtaatcatcacgatgtctatttcaagggtatgccaaccttgaatggtgaagtattgagaagacatggtgacattttcgaattgacctctaacggttactaccatgcacacggtagagccgatgacactatgaacatcggtggtatcaaaattagttctatcgaaatcgaaagagtctgtaatgaagtagatgacagagtttttgaaaccactgctattggtgttccacctttgggtggtggtccagaacaattggtcatatttttcgtattgaaggattcaaacgacacaaccattgatttgaaccaattgagattatcctttaacttgggtttgcaaaagaaattgaacccattattcaaagttactagagttgtcccattgtcatccttacctagaactgcaacaaacaagatcatgagaagagttttgagacaacaattcagtcatttcgaagaaggtagaggttctttgttaacatgtggtgacgttgaagaaaatccaggtcctatggcttcagaaaaggaaataagaagagaaagattcttgaacgtattcccaaagttagttgaagaattgaacgctagtttgttagcttatggtatgcctaaagaagcctgcgattggtatgctcactctttaaactacaatactccaggtggtaaattgaatagaggtttgagtgtagttgatacttatgctatcttgtctaacaaaaccgttgaacaattaggtcaagaagaatacgaaaaggtcgctatcttgggttggtgtattgaattgttgcaagcatactttttggttgccgatgacatgatggataagtctataacaagaagaggtcaaccatgctggtacaaagttccagaagttggtgaaatagccataaatgatgcttttatgttggaagccgctatctataaattgttgaagtcacatttcagaaacgaaaagtactacatcgatattaccgaattattccacgaagttactttccaaacagaattgggtcaattgatggatttgataactgcacctgaagataaagttgacttgtcaaagttttccttgaagaaacattcattcatcgtcacctttgaaactgcttattactccttctatttgccagtcgccttggctatgtacgtagctggtattactgatgaaaaagacttgaagcaagcaagagatgttttgatacctttgggtgaatacttccaaatccaagatgactacttagactgtttcggtactccagaacaaataggtaaaatcggtacagatattcaagacaataagtgcagttgggttattaacaaggctttggaattagcatctgccgaacaaagaaagactttggatgaaaactacggtaaaaaggactcagttgctgaagcaaagtgtaagaaaatttttaatgatttgaagattgaacaattgtaccatgaatacgaagaatccatcgctaaagacttaaaggcaaagattagtcaagttgatgaatcaagaggttttaaagccgacgttttgacagctttcttgaataaggtctacaagagatcaaaggattacaaggatcatgacggtgactataaagaccacgatattgactacaaagatgacgatgacaagtaagcggccgc

SEQ ID NO: 15 - DNA, 1553 bp, synthetic construct
  misc_feature 1..1553: note = Codon Optimized
  source 1..1553: mol_type = other DNA
actagtatgaaccatttgagagccgaaggtcctgcctccgtattagccataggtacagccaacccagaaaacatattgatccaagatgaatttcctgattattacttcagagttaccaagagtgaacacatgactcaattgaaggaaaagtttagaaaaatatgtgataagtctatgatcagaaagagaaactgcttcttgaacgaagaacatttgaagcaaaatccaagattggtagaacacgaaatgcaaacattggatgccagacaagacatgttagttgtcgaagttcctaaattgggtaaagatgcttgtgcaaaagccattaaggaatggggtcaaccaaagtcaaagatcactcatttgatttttacaagtgcatctactacagatatgcctggtgcagactaccactgtgccaaattgttaggtttgtcaccatccgttaagagagtcatgatgtatcaattaggttgctacggtggtggtactgttttgagaatcgctaaggatattgcagaaaacaacaagggtgccagagtattagctgtttgttgcgacattatggcttgcttgtttagaggtccaagtgattctgacttggaattgttagttggtcaagctatcttcggtgacggtgctgctgctgttattgttggtgcagaacctgacgaatctgttggtgaaagaccaatatttgaattagtcagtacaggtcaaaccatcttgcctaattctgaaggtacaattggtggtcatataagagaagcaggtttgatcttcgatttgcacaaagacgttccaatgttaatctctaacaacatagaaaagtgtttgatagaagcattcactcctataggtatctcagattggaactctattttctggataacacatccaggtggtaaagccattttggataaggttgaagaaaaattggatttgaagaaagaaaagtttgtagatagtagacatgttttatctgaacacggtaacatgtcttcatccactgtcttgttcgtaatggatgaattgagaaagagatcattagaagagggtaaatctactactggtgacggttttgaatggggtgtcttatttggtttcggtcctggtttgaccgtcgaaagagtagttgtcagatcagtaccaattaaatatgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtacccttacgatgttccagactatgcataagcggccgc

SEQ ID NO: 16 - DNA, 2918 bp, synthetic construct
  misc_feature 1..2918: note = Codon Optimized
  source 1..2918: mol_type = other DNA
actagtatgggtttatcatccgtctgtactttctccttccaaactaactatcataccttattgaatcctcacaacaacaatccaaaaacatcattgttgtgttacagacatccaaagacacctattaagtactcttacaacaactttccatcaaaacattgttcaaccaagtccttccacttacaaaataagtgctccgaaagtttgtctatagctaagaactctatcagagctgcaactacaaatcaaactgaaccacctgaaagtgataatcactctgttgccacaaaaattttgaacttcggtaaagcatgttggaagttgcaaagaccatacaccataatcgcttttacttcttgtgcatgcggtttattcggtaaagaattgttgcataacactaacttaatttcatggtccttgatgttcaaggcatttttctttttagttgccatcttgtgcatcgcttcattcaccactacaattaatcaaatatacgatttgcacatcgacagaattaacaaaccagatttgcctttggcttcaggtgaaatatccgtcaatactgcatggatcatgtctatcatagtagccttgttcggtttgatcatcacaattaaaatgaagggtggtccattgtacatcttcggttactgtttcggtatcttcggtggtattgtctattccgtaccaccttttagatggaaacaaaaccctagtactgcctttttgttgaatttcttagctcatatcatcacaaacttcaccttctactacgcttcaagagctgctttaggtttgccattcgaattgagaccttcattcacatttttgttggcattcatgaaaagtatgggttctgcattagccttgatcaaggatgcctctgacgttgaaggtgacacaaagttcggtattagtaccttggcttctaagtacggttcaagaaatttgactttgttctgctccggtatcgttttgttaagttacgtcgcagccattttggcaggtatcatttggccacaagcctttaattctaacgttatgttgttgtcacatgccatcttggctttctggttgatcttgcaaactagagatttcgctttgacaaattatgaccctgaagcaggtagaagattctacgagtttatgtggaaattgtactacgctgaatatttggtatacgtttttattgaaggtagaggttctttgttgacctgtggtgacgttgaagaaaatccaggtcctatgaaatgttcaactttctccttttggttcgtatgcaagatcatcttctttttcttttcctttaacatccaaacaagtatcgcaaacccaagagaaaactttttgaagtgcttctcacaatacatacctaataacgccaccaatttgaagttggtttacactcaaaacaacccattgtacatgtccgtcttgaacagtacaatccataatttgagattcacttctgataccactccaaaacctttggtcattgtaacccctagtcatgtatctcacatccaaggtactatcttatgttctaaaaaggttggtttgcaaattagaactagatccggtggtcatgatagtgaaggcatgtcatacatctcccaagttccattcgttatcgttgatttgagaaacatgagatcaattaaaatagacgtacactcacaaactgcttgggttgaagctggtgcaacattgggtgaagtatactactgggttaacgaaaagaatgaaaacttatcattggctgctggttactgtccaacagtttgcgcaggtggtcattttggtggtggtggttatggtcctttaatgagaaactacggtttggccgctgataacataatcgacgctcatttggtaaatgttcacggtaaagttttggatagaaagtctatgggtgaagacttattttgggctttgagaggtggtggtgcagaatcattcggtatcatagttgcttggaagataagattagtcgcagtaccaaagtctactatgttctcagtcaaaaagataatggaaatccatgaattagttaaattggtcaataagtggcaaaacatcgcatacaagtacgataaggacttgttgttgatgactcatttcatcacaagaaacatcaccgataaccaaggtaaaaataagactgctatccacacatacttttcttcagttttcttgggtggtgtcgattccttagtagacttgatgaataagtcttttccagaattaggtattaagaaaactgattgtagacaattgtcttggatcgacaccatcatcttttattcaggtgttgtcaactacgatacagacaacttcaacaaagaaatattattggatagatccgcaggtcaaaacggtgcctttaaaattaagttagactacgttaaaaagccaatacctgaatcagttttcgtccaaatcttagaaaaattgtacgaagaagatattggtgcaggcatgtacgccttgtatccatacggtggtataatggacgaaatcagtgaatctgccattccatttcctcatagagctggtatcttatacgaattgtggtacatttgttcatgggaaaagcaagaagataacgaaaagcacttaaactggattagaaacatctataacttcatgactccatacgtttctaaaaaccctagattggcatatttgaactacagagatttggacatcggtattaacgatccaaagaatcctaacaactatacccaagctagaatttggggtgaaaaatacttcggtaaaaatttcgatagattagtaaaggttaagacattggttgacccaaacaacttctttagaaacgaacaatccattccacctttacctagacatagacacgaacaaaaattaataagtgaagaagatttgtaagcggccgc

SEQ ID NO: 17 - DNA, 167 bp, synthetic construct
  misc_feature 1..167: note = Codon Optimized
  source 1..167: mol_type = other DNA
agccaaaataatgataacgagaataatatcaagaataccttagaacaacatcgacaacaacaacaggcattttcggatatgagtcacgtggagtattccagaattacaaaattttttcaagaacaaccactggagggatatacccttttctctcacaggtctgcgcc

SEQ ID NO: 18 - DNA, 1518 bp, synthetic construct
  misc_feature 1..1518: note = Codon Optimized
  source 1..1518: mol_type = other DNA
atggtttccaatcacttgtttgacgcaatgagagccgctgcccctggtaacgcccctttcataagaatagataatactagaacttggacatacgatgacgcctttgctttatctggtagaatagcatcagctatggatgctttgggtatcagaccaggtgacagagtcgcagttcaagtagaaaaatccgctgaagcattgatcttgtatttggcttgtttgagaagtggtgcagtttatttgccattgaatactgcctacacattagctgaattggattacttcataggtgacgcagaacctagattggttgtagtcgcctcttcagccagagctggtgtagaaacaattgctaaaccaagaggtgcaatagtcgaaaccttagatgctgctggttctggtagtttgttagatttggccagagacgaacctgctgattttgttgacgcttcaagatcagccgatgacttagccgctattttgtacacctctggtactacaggtagatcaaagggtgctatgttgactcatggtaatttgttgtcaaacgcattaaccttgagagatttctggagagttactgccggtgacagattaatccacgctttgccaatttttcatactcacggtttattcgttgctaccaacgtaactttgttagcaggtgcctccatgttcttgttgagtaagttcgatccagaagaaatattatctttgatgcctcaagctactatgttgatgggtgtcccaacattctacgttagattgttacaatcacctagattagataagcaagctgttgcaaacatcagattgtttatatccggtagtgctccattgttagcagaaacccatactgaatttcaagcaagaacaggtcacgccattttagaaagatacggtatgacagaaaccaatatgaacacttctaacccttatgaaggtaaaagaatagctggtacagttggttttccattgcctgatgtcacagttagagtaaccgacccagccactggtttagctttgccacctgaacaaactggtatgatcgaaattaaaggtccaaacgtttttaagggttactggagaatgcctgaaaagactgctgctgagtttactgctgatggtttctttatctctggtgacttaggtaaaattgatagagacggttatgtccatattgttggtcgtggtaaagatttggttatatccggtggttataacatctaccctaaggaagtagaaggtgaaatagatcaaatcgaaggtgttgtagaatcagctgtaataggtgtcccacatcctgattttggtgaaggtgttacagcagtcgttgtaagaaaaccaggtgctgcattagatgaaaaggcaattgtttctgccttacaagacagattggctagatacaagcaaccaaagagaataatcttcgcagaagatttgcctagaaatactatgggtaaagtacaaaagaacatcttgagacaacaatacgccgacttatacaccagaacctga

SEQ ID NO: 19 - DNA, 2092 bp, synthetic construct
  misc_feature 1..2092: note = Codon Optimized
  source 1..2092: mol_type = other DNA
actagtatgggtttatcatccgtctgtactttctccttccaaactaactatcataccttattgaatcctcacaacaacaatccaaaaacatcattgttgtgttacagacatccaaagacacctattaagtactcttacaacaactttccatcaaaacattgttcaaccaagtccttccacttacaaaataagtgctccgaaagtttgtctatagctaagaactctatcagagctgcaactacaaatcaaactgaaccacctgaaagtgataatcactctgttgccacaaaaattttgaacttcggtaaagcatgttggaagttgcaaagaccatacaccataatcgcttttacttcttgtgcatgcggtttattcggtaaagaattgttgcataacactaacttaatttcatggtcctatgaactgctccgcattctctttctggttcgtctgtaaaataatcttcttcttcttgtccttcaacatccaaatctccatcgcaaatccacaagaaaactttttgaagtgtttctccgaatacatcccaaacaaccctgctaacccaaagtttatatatactcaacatgatcaattgtacatgtccgttttgaacagtaccatccaaaatttgagattcacttctgacactacaccaaaacctttagtcattgttacaccttccaatgttagtcacattcaagcttctatattgtgctctaagaaagtaggtttgcaaatcagaactagatcaggtggtcatgatgcagaaggcatgtcttacatctcacaagttccattcgttgtagtcgatttgagaaatatgcattccataaagatcgacgttcacagtcaaacagcatgggtagaagcaggtgccaccttgggtgaagtttactactggatcaacgaaaagaatgaaaacttttctttccctggtggttactgtccaacagtaggtgtcggtggtcacttttctggtggtggttatggtgcattgatgagaaactacggtttagctgcagataatattatagacgcccatttggttaacgtagatggtaaagttttggacagaaagtctatgggtgaagatttgttttgggccataagaggtggtggtggtgaaaatttcggtatcattgccgcttggaaaattaagttagtcgctgttccttccaaaagtactattttctctgtcaaaaagaacatggaaatccacggtttggttaagttgtttaataagtggcaaaacatcgcttacaagtacgataaggacttggttttgatgacccatttcatcactaaaaatattacagataaccatggtaaaaataagaccactgttcacggttatttttcttcaattttccatggtggtgtagattctttggttgatttgatgaataagtcattcccagaattgggtattaaaaagacagattgcaaggaattttcttggatagacacaaccatcttctattcaggtgttgtaaacttcaacaccgctaacttcaaaaaggaaatcttgttggatagatccgctggtaaaaagaccgctttttctattaaattggactacgttaagaaaccaatccctgaaactgcaatggtcaagatattggaaaagttgtacgaagaagatgtaggtgtcggcatgtacgttttgtatccatacggtggtattatggaagaaatatctgaatcagccataccatttcctcacagagctggtatcatgtatgaattatggtacacagcctcatgggaaaagcaagaagataacgaaaagcatatcaactgggtcagatccgtttacaacttcactacaccttacgttagtcaaaacccaagattggcatatttgaactacagagatttggacttaggtaaaactaaccctgaatctccaaataactatacacaagcaagaatttggggtgaaaagtactttggtaaaaatttcaacagattagttaaagtaaagactaaagccgaccctaacaactttttcagaaacgaacaatccatcccacctttgccacctcaccaccacgaacaaaaattaataagtgaagaagatttgtaagtcgac

SEQ ID NO: 20 - DNA, 7904 bp, synthetic construct
  misc_feature 1..7904: note = Codon Optimized
  source 1..7904: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgccgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - DNA, 6773 bp, synthetic construct
  misc_feature 1..6773: note = Codon Optimized
  source 1..6773: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgccgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 22 - DNA, 7690 bp, synthetic construct
  misc_feature 1..7690: note = Codon Optimized
  source 1..7690: mol_type = other DNA
ttatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatggtttccaatcacttgtttgacgcaatgagagccgctgcccctggtaacgcccctttcataagaatagataatactagaacttggacatacgatgacgcctttgctttatctggtagaatagcatcagctatggatgctttgggtatcagaccaggtgacagagtcgcagttcaagtagaaaaatccgctgaagcattgatcttgtatttggcttgtttgagaagtggtgcagtttatttgccattgaatactgcctacacattagctgaattggattacttcataggtgacgcagaacctagattggttgtagtcgcctcttcagccagagctggtgtagaaacaattgctaaaccaagaggtgcaatagtcgaaaccttagatgctgctggttctggtagtttgttagatttggccagagacgaacctgctgattttgttgacgcttcaagatcagccgatgacttagccgctattttgtacacctctggtactacaggtagatcaaagggtgctatgttgactcatggtaatttgttgtcaaacgcattaaccttgagagatttctggagagttactgccggtgacagattaatccacgctttgccaatttttcatactcacggtttattcgttgctaccaacgtaactttgttagcaggtgcctccatgttcttgttgagtaagttcgatccagaagaaatattatctttgatgcctcaagctactatgttgatgggtgtcccaacattctacgttagattgttacaatcacctagattagataagcaagctgttgcaaacatcagattgtttatatccggtagtgctccattgttagcagaaacccatactgaatttcaagcaagaacaggtcacgccattttagaaagatacggtatgacagaaaccaatatgaacacttctaacccttatgaaggtaaaagaatagctggtacagttggttttccattgcctgatgtcacagttagagtaaccgacccagccactggtttagctttgccacctgaacaaactggtatgatcgaaattaaaggtccaaacgtttttaagggttactggagaatgcctgaaaagactgctgctgagtttactgctgatggtttctttatctctggtgacttaggtaaaattgatagagacggttatgtccatattgttggtcgtggtaaagatttggttatatccggtggttataacatctaccctaaggaagtagaaggtgaaatagatcaaatcgaaggtgttgtagaatcagctgtaataggtgtcccacatcctgattttggtgaaggtgttacagcagtcgttgtaagaaaaccaggtgctgcattagatgaaaaggcaattgtttctgccttacaagacagattggctagatacaagcaaccaaagagaataatcttcgcagaagatttgcctagaaatactatgggtaaagtacaaaagaacatcttgagacaacaatacgccgacttatacaccagaaccgaaggtagaggttctttgttaacatgtggtgacgttgaagaaaatccaggtcctatggcttcagaaaaggaaataagaagagaaagattcttgaacgtattcccaaagttagttgaagaattgaacgctagtttgttagcttatggtatgcctaaagaagcctgcgattggtatgctcactctttaaactacaatactccaggtggtaaattgaatagaggtttgagtgtagttgatacttatgctatcttgtctaacaaaaccgttgaacaattaggtcaagaagaatacgaaaaggtcgctatcttgggttggtgtattgaattgttgcaagcatactttttggttgccgatgacatgatggataagtctataacaagaagaggtcaaccatgctggtacaaagttccagaagttggtgaaatagccataaatgatgcttttatgttggaagccgctatctataaattgttgaagtcacatttcagaaacgaaaagtactacatcgatattaccgaattattccacgaagttactttccaaacagaattgggtcaattgatggatttgataactgcacctgaagataaagttgacttgtcaaagttttccttgaagaaacattcattcatcgtcacctttgaaactgcttattactccttctatttgccagtcgccttggctatgtacgtagctggtattactgatgaaaaagacttgaagcaagcaagagatgttttgatacctttgggtgaatacttccaaatccaagatgactacttagactgtttcggtactccagaacaaataggtaaaatcggtacagatattcaagacaataagtgcagttgggttattaacaaggctttggaattagcatctgccgaacaaagaaagactttggatgaaaactacggtaaaaaggactcagttgctgaagcaaagtgtaagaaaatttttaatgatttgaagattgaacaattgtaccatgaatacgaagaatccatcgctaaagacttaaaggcaaagattagtcaagttgatgaatcaagaggttttaaagccgacgttttgacagctttcttgaataaggtctacaagagatcaaagtgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccatt

SEQ ID NO: 23 - DNA, 2163 bp, synthetic construct
  misc_feature 1..2163: note = Codon Optimized
  source 1..2163: mol_type = other DNA
atgggtaaaaactataagtccttggattcagtcgttgcctcagatttcatcgcattgggtatcacctcagaagtagcagaaacattacatggtagattggcagaaatcgtttgtaattatggtgctgcaacccctcaaacttggatcaacatcgctaaccatatcttgtcaccagatttgcctttctccttacaccaaatgttgttttatggttgctacaaggatttcggtccagccccacctgcttggattccagaccctgaaaaagtcaagtcaactaatttgggtgctttgttggaaaagagaggtaaagaatttttgggtgtaaagtacaaagatccaatttcttctttttctcacttccaagaattttctgttagaaaccctgaagtctattggagaacagtattgatggatgaaatgaaaattagtttctctaaggacccagaatgtatcttgagaagagatgacatcaacaacccaggtggttctgaatggttacctggtggttacttgaactcagctaaaaattgcttgaacgtaaactccaataagaaattgaacgatactatgatcgtttggagagacgagggtaacgatgacttgcctttgaataagttgacattagatcaattgagaaagagagtttggttggttggttatgcattggaagaaatgggtttagaaaaaggttgtgcaatagccatcgatatgccaatgcatgttgatgctgttgttatatatttggccatagtattggctggttacgtagttgtctctatagcagattcattttccgcccctgaaatctcaactagattgagattatccaaagctaaggcaattttcacacaagatcacatcatcagaggtaaaaagagaataccattgtattcaagagtagttgaagctaaatccccaatggcaatagttatcccttgtagtggttctaacattggtgcagaattgagagatggtgacatatcttgggattactttttagaaagagccaaggagtttaaaaactgcgagtttactgccagagaacaacctgttgatgcttatactaacatcttattctccagtggtactacaggtgaaccaaaagcaattccttggacacaagccaccccattgaaggctgctgctgatggttggtctcatttggatattagaaaaggtgacgttatagtatggccaactaatttgggttggatgatgggtccttggttggtttatgctagtttgttaaatggtgcatctattgccttgtacaacggtagtcctttagtctctggtttcgctaaatttgttcaagatgcaaaggtcacaatgttgggtgtcgtaccatctattgtaagatcatggaaatccacaaattgtgtttcaggttacgattggtccaccataagatgcttttcttcatccggtgaagcctctaatgtagacgaatatttgtggttaatgggtagagctaactacaagccagttatagaaatgtgtggtggtacagaaatcggtggtgctttttctgctggttcatttttgcaagctcaatctttaagttctttttcatcccaatgtatgggttgcaccttgtacatattagataagaacggttacccaatgcctaaaaataagccaggtatcggtgaattggcattaggtcctgttatgtttggtgcctcaaaaacattgttaaacggtaatcatcacgatgtctatttcaagggtatgccaaccttgaatggtgaagtattgagaagacatggtgacattttcgaattgacctctaacggttactaccatgcacacggtagagccgatgacactatgaacatcggtggtatcaaaattagttctatcgaaatcgaaagagtctgtaatgaagtagatgacagagtttttgaaaccactgctattggtgttccacctttgggtggtggtccagaacaattggtcatatttttcgtattgaaggattcaaacgacacaaccattgatttgaaccaattgagattatcctttaacttgggtttgcaaaagaaattgaacccattattcaaagttactagagttgtcccattgtcatccttacctagaactgcaacaaacaagatcatgagaagagttttgagacaacaattcagtcatttcgaatga

SEQ ID NO: 24 - DNA, 1059 bp, synthetic construct
  misc_feature 1..1059: note = Codon Optimized
  source 1..1059: mol_type = other DNA
atggcttcagaaaaggaaataagaagagaaagattcttgaacgtattcccaaagttagttgaagaattgaacgctagtttgttagcttatggtatgcctaaagaagcctgcgattggtatgctcactctttaaactacaatactccaggtggtaaattgaatagaggtttgagtgtagttgatacttatgctatcttgtctaacaaaaccgttgaacaattaggtcaagaagaatacgaaaaggtcgctatcttgggttggtgtattgaattgttgcaagcatactttttggttgccgatgacatgatggataagtctataacaagaagaggtcaaccatgctggtacaaagttccagaagttggtgaaatagccataaatgatgcttttatgttggaagccgctatctataaattgttgaagtcacatttcagaaacgaaaagtactacatcgatattaccgaattattccacgaagttactttccaaacagaattgggtcaattgatggatttgataactgcacctgaagataaagttgacttgtcaaagttttccttgaagaaacattcattcatcgtcacctttgaaactgcttattactccttctatttgccagtcgccttggctatgtacgtagctggtattactgatgaaaaagacttgaagcaagcaagagatgttttgatacctttgggtgaatacttccaaatccaagatgactacttagactgtttcggtactccagaacaaataggtaaaatcggtacagatattcaagacaataagtgcagttgggttattaacaaggctttggaattagcatctgccgaacaaagaaagactttggatgaaaactacggtaaaaaggactcagttgctgaagcaaagtgtaagaaaatttttaatgatttgaagattgaacaattgtaccatgaatacgaagaatccatcgctaaagacttaaaggcaaagattagtcaagttgatgaatcaagaggttttaaagccgacgttttgacagctttcttgaataaggtctacaagagatcaaagtag

SEQ ID NO: 25 - DNA, 1158 bp, synthetic construct
  misc_feature 1..1158: note = Codon Optimized
  source 1..1158: mol_type = other DNA
atgaaccatttgagagccgaaggtcctgcctccgtattagccataggtacagccaacccagaaaacatattgatccaagatgaatttcctgattattacttcagagttaccaagagtgaacacatgactcaattgaaggaaaagtttagaaaaatatgtgataagtctatgatcagaaagagaaactgcttcttgaacgaagaacatttgaagcaaaatccaagattggtagaacacgaaatgcaaacattggatgccagacaagacatgttagttgtcgaagttcctaaattgggtaaagatgcttgtgcaaaagccattaaggaatggggtcaaccaaagtcaaagatcactcatttgatttttacaagtgcatctactacagatatgcctggtgcagactaccactgtgccaaattgttaggtttgtcaccatccgttaagagagtcatgatgtatcaattaggttgctacggtggtggtactgttttgagaatcgctaaggatattgcagaaaacaacaagggtgccagagtattagctgtttgttgcgacattatggcttgcttgtttagaggtccaagtgattctgacttggaattgttagttggtcaagctatcttcggtgacggtgctgctgctgttattgttggtgcagaacctgacgaatctgttggtgaaagaccaatatttgaattagtcagtacaggtcaaaccatcttgcctaattctgaaggtacaattggtggtcatataagagaagcaggtttgatcttcgatttgcacaaagacgttccaatgttaatctctaacaacatagaaaagtgtttgatagaagcattcactcctataggtatctcagattggaactctattttctggataacacatccaggtggtaaagccattttggataaggttgaagaaaaattggatttgaagaaagaaaagtttgtagatagtagacatgttttatctgaacacggtaacatgtcttcatccactgtcttgttcgtaatggatgaattgagaaagagatcattagaagagggtaaatctactactggtgacggttttgaatggggtgtcttatttggtttcggtcctggtttgaccgtcgaaagagtagttgtcagatcagtaccaattaaatattag

SEQ ID NO: 26 - DNA, 315 bp, synthetic construct
  misc_feature 1..315: note = Codon Optimized
  source 1..315: mol_type = other DNA
atggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagttaaagccaaaataa

SEQ ID NO: 27 - DNA, 1188 bp, synthetic construct
  misc_feature 1..1188: note = Codon Optimized
  source 1..1188: mol_type = other DNA
atgggtttatcatccgtctgtactttctccttccaaactaactatcataccttattgaatcctcacaacaacaatccaaaaacatcattgttgtgttacagacatccaaagacacctattaagtactcttacaacaactttccatcaaaacattgttcaaccaagtccttccacttacaaaataagtgctccgaaagtttgtctatagctaagaactctatcagagctgcaactacaaatcaaactgaaccacctgaaagtgataatcactctgttgccacaaaaattttgaacttcggtaaagcatgttggaagttgcaaagaccatacaccataatcgcttttacttcttgtgcatgcggtttattcggtaaagaattgttgcataacactaacttaatttcatggtccttgatgttcaaggcatttttctttttagttgccatcttgtgcatcgcttcattcaccactacaattaatcaaatatacgatttgcacatcgacagaattaacaaaccagatttgcctttggcttcaggtgaaatatccgtcaatactgcatggatcatgtctatcatagtagccttgttcggtttgatcatcacaattaaaatgaagggtggtccattgtacatcttcggttactgtttcggtatcttcggtggtattgtctattccgtaccaccttttagatggaaacaaaaccctagtactgcctttttgttgaatttcttagctcatatcatcacaaacttcaccttctactacgcttcaagagctgctttaggtttgccattcgaattgagaccttcattcacatttttgttggcattcatgaaaagtatgggttctgcattagccttgatcaaggatgcctctgacgttgaaggtgacacaaagttcggtattagtaccttggcttctaagtacggttcaagaaatttgactttgttctgctccggtatcgttttgttaagttacgtcgcagccattttggcaggtatcatttggccacaagcctttaattctaacgttatgttgttgtcacatgccatcttggctttctggttgatcttgcaaactagagatttcgctttgacaaattatgaccctgaagcaggtagaagattctacgagtttatgtggaaattgtactacgctgaatatttggtatacgtttttatttag

SEQ ID NO: 28 - DNA, 1635 bp, synthetic construct
  misc_feature 1..1635: note = Codon Optimized
  source 1..1635: mol_type = other DNA
atgaaatgttcaactttctccttttggttcgtatgcaagatcatcttctttttcttttcctttaacatccaaacaagtatcgcaaacccaagagaaaactttttgaagtgcttctcacaatacatacctaataacgccaccaatttgaagttggtttacactcaaaacaacccattgtacatgtccgtcttgaacagtacaatccataatttgagattcacttctgataccactccaaaacctttggtcattgtaacccctagtcatgtatctcacatccaaggtactatcttatgttctaaaaaggttggtttgcaaattagaactagatccggtggtcatgatagtgaaggcatgtcatacatctcccaagttccattcgttatcgttgatttgagaaacatgagatcaattaaaatagacgtacactcacaaactgcttgggttgaagctggtgcaacattgggtgaagtatactactgggttaacgaaaagaatgaaaacttatcattggctgctggttactgtccaacagtttgcgcaggtggtcattttggtggtggtggttatggtcctttaatgagaaactacggtttggccgctgataacataatcgacgctcatttggtaaatgttcacggtaaagttttggatagaaagtctatgggtgaagacttattttgggctttgagaggtggtggtgcagaatcattcggtatcatagttgcttggaagataagattagtcgcagtaccaaagtctactatgttctcagtcaaaaagataatggaaatccatgaattagttaaattggtcaataagtggcaaaacatcgcatacaagtacgataaggacttgttgttgatgactcatttcatcacaagaaacatcaccgataaccaaggtaaaaataagactgctatccacacatacttttcttcagttttcttgggtggtgtcgattccttagtagacttgatgaataagtcttttccagaattaggtattaagaaaactgattgtagacaattgtcttggatcgacaccatcatcttttattcaggtgttgtcaactacgatacagacaacttcaacaaagaaatattattggatagatccgcaggtcaaaacggtgcctttaaaattaagttagactacgttaaaaagccaatacctgaatcagttttcgtccaaatcttagaaaaattgtacgaagaagatattggtgcaggcatgtacgccttgtatccatacggtggtataatggacgaaatcagtgaatctgccattccatttcctcatagagctggtatcttatacgaattgtggtacatttgttcatgggaaaagcaagaagataacgaaaagcacttaaactggattagaaacatctataacttcatgactccatacgtttctaaaaaccctagattggcatatttgaactacagagatttggacatcggtattaacgatccaaagaatcctaacaactatacccaagctagaatttggggtgaaaaatacttcggtaaaaatttcgatagattagtaaaggttaagacattggttgacccaaacaacttctttagaaacgaacaatccattccacctttacctagacatagacactga

SEQ ID NO: 29 - DNA, 1638 bp, synthetic construct
  misc_feature 1..1638: note = Codon Optimized
  source 1..1638: mol_type = other DNA
atgaactgctccgcattctctttctggttcgtctgtaaaataatcttcttcttcttgtccttcaacatccaaatctccatcgcaaatccacaagaaaactttttgaagtgtttctccgaatacatcccaaacaaccctgctaacccaaagtttatatatactcaacatgatcaattgtacatgtccgttttgaacagtaccatccaaaatttgagattcacttctgacactacaccaaaacctttagtcattgttacaccttccaatgttagtcacattcaagcttctatattgtgctctaagaaagtaggtttgcaaatcagaactagatcaggtggtcatgatgcagaaggcatgtcttacatctcacaagttccattcgttgtagtcgatttgagaaatatgcattccataaagatcgacgttcacagtcaaacagcatgggtagaagcaggtgccaccttgggtgaagtttactactggatcaacgaaaagaatgaaaacttttctttccctggtggttactgtccaacagtaggtgtcggtggtcacttttctggtggtggttatggtgcattgatgagaaactacggtttagctgcagataatattatagacgcccatttggttaacgtagatggtaaagttttggacagaaagtctatgggtgaagatttgttttgggccataagaggtggtggtggtgaaaatttcggtatcattgccgcttggaaaattaagttagtcgctgttccttccaaaagtactattttctctgtcaaaaagaacatggaaatccacggtttggttaagttgtttaataagtggcaaaacatcgcttacaagtacgataaggacttggttttgatgacccatttcatcactaaaaatattacagataaccatggtaaaaataagaccactgttcacggttatttttcttcaattttccatggtggtgtagattctttggttgatttgatgaataagtcattcccagaattgggtattaaaaagacagattgcaaggaattttcttggatagacacaaccatcttctattcaggtgttgtaaacttcaacaccgctaacttcaaaaaggaaatcttgttggatagatccgctggtaaaaagaccgctttttctattaaattggactacgttaagaaaccaatccctgaaactgcaatggtcaagatattggaaaagttgtacgaagaagatgtaggtgtcggcatgtacgttttgtatccatacggtggtattatggaagaaatatctgaatcagccataccatttcctcacagagctggtatcatgtatgaattatggtacacagcctcatgggaaaagcaagaagataacgaaaagcatatcaactgggtcagatccgtttacaacttcactacaccttacgttagtcaaaacccaagattggcatatttgaactacagagatttggacttaggtaaaactaaccctgaatctccaaataactatacacaagcaagaatttggggtgaaaagtactttggtaaaaatttcaacagattagttaaagtaaagactaaagccgaccctaacaactttttcagaaacgaacaatccatcccacctttgccacctcaccaccactaa

SEQ ID NO: 30 - DNA, 960 bp, synthetic construct
  misc_feature 1..960: note = Codon Optimized
  source 1..960: mol_type = other DNA
atggctaatcaaacagaacctccagaatctaatacgaaatatagtgtagttaccaaaatcctaagttttggccacacttgttggaaattgcagagaccgtatactttcattggagtgattagttgcgcctgtggattgttcggtagagagttattccataatactaatttgctatcatggtctctgatgttgaaagctttcagctcattgatggtaatactgtcagtgaatctatgtaccaatatcataaaccagatcactgacctggacatagacagaatcaataagccggacttgccattggcgagcggggaaatgtccattgaaacagcatggattatgagtattatagttgcactaactggattgatacttacgataaagcttaattgcggccctttgtttatatctctatattgtgtcagcatactagtcggggcactatattcagtaccgccattcagatggaagcaaaatcccaataccgcattctcaagttattttatgggactggtgatcgtcaattttacctgctattacgcaagcagggccgcctttggactgccattcgagatgtcacccccgttcacattcattcttgcctttgtcaagtcaatgggtagcgcactttttttgtgtaaagatgtctctgacattgaaggagattctaagcacggtatatctacccttgcgacgaggtatggagcaaaaaacattactttcctttgctcaggaatcgtactgctaacctacgtaagcgcgatattggctgcgattatttggccacaagccttcaagtccaacgtgatgctgttgagtcacgcaaccctggccttttggcttatctttcagactagagagttcgcgttaactaattacaatccagaggcagggaggaagttttacgagttcatgtggaagctgcactacgctgaatacttagtctatgtatttatatag

SEQ ID NO: 31 - DNA, 960 bp, synthetic construct; identical to SEQ ID NO: 30
  misc_feature 1..960: note = Codon Optimized
  source 1..960: mol_type = other DNA

SEQ ID NO: 32 - DNA, 1023 bp, synthetic construct
  misc_feature 1..1023: note = Codon Optimized
  source 1..1023: mol_type = other DNA
atgagattcccatctattttcactgctgttttgttcgctgcttcttctgctttggctgctccagttgctaatcaaacagaacctccagaatctaatacgaaatatagtgtagttaccaaaatcctaagttttggccacacttgttggaaattgcagagaccgtatactttcattggagtgattagttgcgcctgtggattgttcggtagagagttattccataatactaatttgctatcatggtctctgatgttgaaagctttcagctcattgatggtaatactgtcagtgaatctatgtaccaatatcataaaccagatcactgacctggacatagacagaatcaataagccggacttgccattggcgagcggggaaatgtccattgaaacagcatggattatgagtattatagttgcactaactggattgatacttacgataaagcttaattgcggccctttgtttatatctctatattgtgtcagcatactagtcggggcactatattcagtaccgccattcagatggaagcaaaatcccaataccgcattctcaagttattttatgggactggtgatcgtcaattttacctgctattacgcaagcagggccgcctttggactgccattcgagatgtcacccccgttcacattcattcttgcctttgtcaagtcaatgggtagcgcactttttttgtgtaaagatgtctctgacattgaaggagattctaagcacggtatatctacccttgcgacgaggtatggagcaaaaaacattactttcctttgctcaggaatcgtactgctaacctacgtaagcgcgatattggctgcgattatttggccacaagccttcaagtccaacgtgatgctgttgagtcacgcaaccctggccttttggcttatctttcagactagagagttcgcgttaactaattacaatccagaggcagggaggaagttttacgagttcatgtggaagctgcactacgctgaatacttagtctatgtatttatatag

SEQ ID NO: 33 - DNA, 6025 bp, synthetic construct
  misc_feature 1..6025: note = Codon Optimized
  source 1..6025: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatggctaatcaaacagaacctccagaatctaatacgaaatatagtgtagttaccaaaatcctaagttttggccacacttgttggaaattgcagagaccgtatactttcattggagtgattagttgcgcctgtggattgttcggtagagagttattccataatactaatttgctatcatggtctctgatgttgaaagctttcagctcattgatggtaatactgtcagtgaatctatgtaccaatatcataaaccagatcactgacctggacatagacagaatcaataagccggacttgccattggcgagcggggaaatgtccattgaaacagcatggattatgagtattatagttgcactaactggattgatacttacgataaagcttaattgcggccctttgtttatatctctatattgtgtcagcatactagtcggggcactatattcagtaccgccattcagatggaagcaaaatcccaataccgcattctcaagttattttatgggactggtgatcgtcaattttacctgctattacgcaagcagggccgcctttggactgccattcgagatgtcacccccgttcacattcattcttgcctttgtcaagtcaatgggtagcgcactttttttgtgtaaagatgtctctgacattgaaggagattctaagcacggtatatctacccttgcgacgaggtatggagcaaaaaacattactttcctttgctcaggaatcgtactgctaacctacgtaagcgcgatattggctgcgattatttggccacaagccttcaagtccaacgtgatgctgttgagtcacgcaaccctggccttttggcttatctttcagactagagagttcgcgttaactaattacaatccagaggcagggaggaagttttacgagttcatgtggaagctgcactacgctgaatacttagtctatgtatttatataggatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 34 - DNA, 6025 bp, synthetic construct; identical to SEQ ID NO: 33
  misc_feature 1..6025: note = Codon Optimized
  source 1..6025: mol_type = other DNA

SEQ ID NO: 35 - DNA, 6088 bp, synthetic construct
  misc_feature 1..6088: note = Codon Optimized
  source 1..6088: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatgagattcccatctattttcactgctgttttgttcgctgcttcttctgctttggctgctccagttgctaatcaaacagaacctccagaatctaatacgaaatatagtgtagttaccaaaatcctaagttttggccacacttgttggaaattgcagagaccgtatactttcattggagtgattagttgcgcctgtggattgttcggtagagagttattccataatactaatttgctatcatggtctctgatgttgaaagctttcagctcattgatggtaatactgtcagtgaatctatgtaccaatatcataaaccagatcactgacctggacatagacagaatcaataagccggacttgccattggcgagcggggaaatgtccattgaaacagcatggattatgagtattatagttgcactaactggattgatacttacgataaagcttaattgcggccctttgtttatatctctatattgtgtcagcatactagtcggggcactatattcagtaccgccattcagatggaagcaaaatcccaataccgcattctcaagttattttatgggactggtgatcgtcaattttacctgctattacgcaagcagggccgcctttggactgccattcgagatgtcacccccgttcacattcattcttgcctttgtcaagtcaatgggtagcgcactttttttgtgtaaagatgtctctgacattgaaggagattctaagcacggtatatctacccttgcgacgaggtatggagcaaaaaacattactttcctttgctcaggaatcgtactgctaacctacgtaagcgcgatattggctgcgattatttggccacaagccttcaagtccaacgtgatgctgttgagtcacgcaaccctggccttttggcttatctttcagactagagagttcgcgttaactaattacaatccagaggcagggaggaagttttacgagttcatgtggaagctgcactacgctgaatacttagtctatgtatttatataggatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 36 - DNA, 1197 bp, synthetic construct
  misc_feature 1..1197: note = Codon Optimized
  source 1..1197: mol_type = other DNA
atgggccttagtctagtttgtacgttttcttttcagacgaactaccacacgttgctgaatccgcacaataagaacccaaaaaactctctattgagctatcagcatcctaagaccccgataataaagtcttcttacgataactttccttctaaatactgtttaacaaagaactttcatttactgggactgaactctcataaccgtataagtagccaatccaggagcattcgtgcgggctcagatcagattgaaggttccccccaccacgaaagtgacaactccatagcgaccaaaattcttaattttgggcacacttgttggaagttacaaagaccctacgtagtgaaaggtatgatatccatagcgtgtggtctgtttggccgtgaattgtttaataacagacacttattcagttgggggttaatgtggaaggctttttttgctctagttcccatcttgagttttaacttcttcgcggctataatgaaccaaatctacgacgtcgacatcgacaggattaataaaccggatcttccacttgtgtccggagaaatgtccattgaaacggcttggatccttagtattattgttgcccttactggtcttattgtgaccattaagctaaaatcagccccactttttgtttttatctatatatttggcatctttgccggattcgcgtattcagttccccctatccgttggaaacaatacccctttacgaacttccttataacaatttcctcccatgttgggttggccttcacatcatactcagcgacaacttcagcactaggattgcccttcgtgtggaggcccgcatttagctttatcattgcttttatgacggttatgggcatgacaatagcattcgccaaagacattagtgatattgaaggtgatgcaaaatacggtgtgtcaacggtcgccactaagttaggagcaagaaatatgacattcgtggtgtcaggtgttttattgcttaattatcttgtgtccattagcatcggtataatctggcctcaggttttcaaatcaaatatcatgatcctatctcacgcaattctagctttctgcttgatctttcagacgagagaattggctctagccaactacgcatcagcacctagtaggcagttcttcgaatttatttggctattgtattacgcagagtatttcgtgtatgtgttcatttaa

SEQ ID NO: 37 - DNA, 969 bp, synthetic construct
  misc_feature 1..969: note = Codon Optimized
  source 1..969: mol_type = other DNA
atggcgggctcagatcagattgaaggttccccccaccacgaaagtgacaactccatagcgaccaaaattcttaattttgggcacacttgttggaagttacaaagaccctacgtagtgaaaggtatgatatccatagcgtgtggtctgtttggccgtgaattgtttaataacagacacttattcagttgggggttaatgtggaaggctttttttgctctagttcccatcttgagttttaacttcttcgcggctataatgaaccaaatctacgacgtcgacatcgacaggattaataaaccggatcttccacttgtgtccggagaaatgtccattgaaacggcttggatccttagtattattgttgcccttactggtcttattgtgaccattaagctaaaatcagccccactttttgtttttatctatatatttggcatctttgccggattcgcgtattcagttccccctatccgttggaaacaatacccctttacgaacttccttataacaatttcctcccatgttgggttggccttcacatcatactcagcgacaacttcagcactaggattgcccttcgtgtggaggcccgcatttagctttatcattgcttttatgacggttatgggcatgacaatagcattcgccaaagacattagtgatattgaaggtgatgcaaaatacggtgtgtcaacggtcgccactaagttaggagcaagaaatatgacattcgtggtgtcaggtgttttattgcttaattatcttgtgtccattagcatcggtataatctggcctcaggttttcaaatcaaatatcatgatcctatctcacgcaattctagctttctgcttgatctttcagacgagagaattggctctagccaactacgcatcagcacctagtaggcagttcttcgaatttatttggctattgtattacgcagagtatttcgtgtatgtgttcatttaa

SEQ ID NO: 38 - DNA, 1032 bp, synthetic construct
  misc_feature 1..1032: note = Codon Optimized
  source 1..1032: mol_type = other DNA
atgagattcccatctattttcactgctgttttgttcgctgcttcttctgctttggctgctccagttgcgggctcagatcagattgaaggttccccccaccacgaaagtgacaactccatagcgaccaaaattcttaattttgggcacacttgttggaagttacaaagaccctacgtagtgaaaggtatgatatccatagcgtgtggtctgtttggccgtgaattgtttaataacagacacttattcagttgggggttaatgtggaaggctttttttgctctagttcccatcttgagttttaacttcttcgcggctataatgaaccaaatctacgacgtcgacatcgacaggattaataaaccggatcttccacttgtgtccggagaaatgtccattgaaacggcttggatccttagtattattgttgcccttactggtcttattgtgaccattaagctaaaatcagccccactttttgtttttatctatatatttggcatctttgccggattcgcgtattcagttccccctatccgttggaaacaatacccctttacgaacttccttataacaatttcctcccatgttgggttggccttcacatcatactcagcgacaacttcagcactaggattgcccttcgtgtggaggcccgcatttagctttatcattgcttttatgacggttatgggcatgacaatagcattcgccaaagacattagtgatattgaaggtgatgcaaaatacggtgtgtcaacggtcgccactaagttaggagcaagaaatatgacattcgtggtgtcaggtgttttattgcttaattatcttgtgtccattagcatcggtataatctggcctcaggttttcaaatcaaatatcatgatcctatctcacgcaattctagctttctgcttgatctttcagacgagagaattggctctagccaactacgcatcagcacctagtaggcagttcttcgaatttatttggctattgtattacgcagagtatttcgtgtatgtgttcatttaa

SEQ ID NO: 39 - DNA, 6262 bp, synthetic construct
  misc_feature 1..6262: note = Codon Optimized
  source 1..6262: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatgggccttagtctagtttgtacgttttcttttcagacgaactaccacacgttgctgaatccgcacaataagaacccaaaaaactctctattgagctatcagcatcctaagaccccgataataaagtcttcttacgataactttccttctaaatactgtttaacaaagaactttcatttactgggactgaactctcataaccgtataagtagccaatccaggagcattcgtgcgggctcagatcagattgaaggttccccccaccacgaaagtgacaactccatagcgaccaaaattcttaattttgggcacacttgttggaagttacaaagaccctacgtagtgaaaggtatgatatccatagcgtgtggtctgtttggccgtgaattgtttaataacagacacttattcagttgggggttaatgtggaaggctttttttgctctagttcccatcttgagttttaacttcttcgcggctataatgaaccaaatctacgacgtcgacatcgacaggattaataaaccggatcttccacttgtgtccggagaaatgtccattgaaacggcttggatccttagtattattgttgcccttactggtcttattgtgaccattaagctaaaatcagccccactttttgtttttatctatatatttggcatctttgccggattcgcgtattcagttccccctatccgttggaaacaatacccctttacgaacttccttataacaatttcctcccatgttgggttggccttcacatcatactcagcgacaacttcagcactaggattgcccttcgtgtggaggcccgcatttagctttatcattgcttttatgacggttatgggcatgacaatagcattcgccaaagacattagtgatattgaaggtgatgcaaaatacggtgtgtcaacggtcgccactaagttaggagcaagaaatatgacattcgtggtgtcaggtgttttattgcttaattatcttgtgtccattagcatcggtataatctggcctcaggttttcaaatcaaatatcatgatcctatctcacgcaattctagctttctgcttgatctttcagacgagagaattggctctagccaactacgcatcagcacctagtaggcagttcttcgaatttatttggctattgtattacgcagagtatttcgtgtatgtgttcatttaagatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 40 - DNA, 6034 bp, synthetic construct
  misc_feature 1..6034: note = Codon Optimized
  source 1..6034: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatggcgggctcagatcagattgaaggttccccccaccacgaaagtgacaactccatagcgaccaaaattcttaattttgggcacacttgttggaagttacaaagaccctacgtagtgaaaggtatgatatccatagcgtgtggtctgtttggccgtgaattgtttaataacagacacttattcagttgggggttaatgtggaaggctttttttgctctagttcccatcttgagttttaacttcttcgcggctataatgaaccaaatctacgacgtcgacatcgacaggattaataaaccggatcttccacttgtgtccggagaaatgtccattgaaacggcttggatccttagtattattgttgcccttactggtcttattgtgaccattaagctaaaatcagccccactttttgtttttatctatatatttggcatctttgccggattcgcgtattcagttccccctatccgttggaaacaatacccctttacgaacttccttataacaatttcctcccatgttgggttggccttcacatcatactcagcgacaacttcagcactaggattgcccttcgtgtggaggcccgcatttagctttatcattgcttttatgacggttatgggcatgacaatagcattcgccaaagacattagtgatattgaaggtgatgcaaaatacggtgtgtcaacggtcgccactaagttaggagcaagaaatatgacattcgtggtgtcaggtgttttattgcttaattatcttgtgtccattagcatcggtataatctggcctcaggttttcaaatcaaatatcatgatcctatctcacgcaattctagctttctgcttgatctttcagacgagagaattggctctagccaactacgcatcagcacctagtaggcagttcttcgaatttatttggctattgtattacgcagagtatttcgtgtatgtgttcatttaagatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 41 - DNA, 6088 bp, synthetic construct; identical to SEQ ID NO: 35
  misc_feature 1..6088: note = Codon Optimized
  source 1..6088: mol_type = other DNA

SEQ ID NO: 42 - DNA, 1203 bp, synthetic construct
  misc_feature 1..1203: note = Codon Optimized
  source 1..1203: mol_type = other DNA
atggtattttcctcagtgtgtagttttccgtcctctcttggtacaaactttaagctggtgcctagatctaattttaaggcttcaagttcacattaccacgaaatcaacaatttcattaacaacaaacccattaaatttagttatttctcttcaaggttgtattgcagtgccaagccaatagtacacagagaaaacaagttcacaaaatcattctcactatcacacttacaacgtaaatcttctatcaaggcccatggagagatagaggctgatggaagtaacgggacttctgagttcaacgtaatgaagtccggaaatgctatctggagatttgtgaggccgtatgccgctaaaggtgtcctgtttaactccgcggcaatgttcgctaaggaacttgttggaaatctgaacttatttagctggccgttgatgttcaagatcctttcatttactcttgtcattctgtgtatctttgtatctacatcaggcataaatcagatatatgatctagacatcgatagactgaacaaaccgaacttgcccgtggcaagcggggaaattagcgtagaattggcatggttacttactatagtatgtacgattagtggacttaccttaaccattataactaatagtggccccttttttccgttcctttactcagcctccatattctttggtttcctatactccgcccccccgttccgttggaagaaaaacccctttaccgcctgcttttgcaatgtgatgttatacgtgggaaccagtgttggggtttattatgcctgcaaagccagtttgggccttcctgccaattggtctccagcattctgccttttattttggtttattagtctgctttccatacctatcagcatagctaaggatttatctgatattgaaggtgataggaagtttggaatcattactttctctactaagttcggggcaaaaccgatcgcgtacatatgtcacgggcttatgcttttgaattacgtgagtgttatggccgcggccataatatggcctcaattcttcaactcctcagtaatactgttatcacatgccttcatggcgatctgggttttgtaccaagcgtggatactggagaaaagtaactatgcaacggaaacttgccagaaatattacatcttcttatggataatattctcccttgagcacgctttttacctattcatgtag

SEQ ID NO: 43 - DNA, 1128 bp, synthetic construct
  misc_feature 1..1128: note = Codon Optimized
  source 1..1128: mol_type = other DNA
atggcttcaagttcacattaccacgaaatcaacaatttcattaacaacaaacccattaaatttagttatttctcttcaaggttgtattgcagtgccaagccaatagtacacagagaaaacaagttcacaaaatcattctcactatcacacttacaacgtaaatcttctatcaaggcccatggagagatagaggctgatggaagtaacgggacttctgagttcaacgtaatgaagtccggaaatgctatctggagatttgtgaggccgtatgccgctaaaggtgtcctgtttaactccgcggcaatgttcgctaaggaacttgttggaaatctgaacttatttagctggccgttgatgttcaagatcctttcatttactcttgtcattctgtgtatctttgtatctacatcaggcataaatcagatatatgatctagacatcgatagactgaacaaaccgaacttgcccgtggcaagcggggaaattagcgtagaattggcatggttacttactatagtatgtacgattagtggacttaccttaaccattataactaatagtggccccttttttccgttcctttactcagcctccatattctttggtttcctatactccgcccccccgttccgttggaagaaaaacccctttaccgcctgcttttgcaatgtgatgttatacgtgggaaccagtgttggggtttattatgcctgcaaagccagtttgggccttcctgccaattggtctccagcattctgccttttattttggtttattagtctgctttccatacctatcagcatagctaaggatttatctgatattgaaggtgataggaagtttggaatcattactttctctactaagttcggggcaaaaccgatcgcgtacatatgtcacgggcttatgcttttgaattacgtgagtgttatggccgcggccataatatggcctcaattcttcaactcctcagtaatactgttatcacatgccttcatggcgatctgggttttgtaccaagcgtggatactggagaaaagtaactatgcaacggaaacttgccagaaatattacatcttcttatggataatattctcccttgagcacgctttttacctattcatgtag

SEQ ID NO: 44 - DNA, 1191 bp, synthetic construct
  misc_feature 1..1191: note = Codon Optimized
  source 1..1191: mol_type = other DNA
atgagattcccatctattttcactgctgttttgttcgctgcttcttctgctttggctgctccagttgcttcaagttcacattaccacgaaatcaacaatttcattaacaacaaacccattaaatttagttatttctcttcaaggttgtattgcagtgccaagccaatagtacacagagaaaacaagttcacaaaatcattctcactatcacacttacaacgtaaatcttctatcaaggcccatggagagatagaggctgatggaagtaacgggacttctgagttcaacgtaatgaagtccggaaatgctatctggagatttgtgaggccgtatgccgctaaaggtgtcctgtttaactccgcggcaatgttcgctaaggaacttgttggaaatctgaacttatttagctggccgttgatgttcaagatcctttcatttactcttgtcattctgtgtatctttgtatctacatcaggcataaatcagatatatgatctagacatcgatagactgaacaaaccgaacttgcccgtggcaagcggggaaattagcgtagaattggcatggttacttactatagtatgtacgattagtggacttaccttaaccattataactaatagtggccccttttttccgttcctttactcagcctccatattctttggtttcctatactccgcccccccgttccgttggaagaaaaacccctttaccgcctgcttttgcaatgtgatgttatacgtgggaaccagtgttggggtttattatgcctgcaaagccagtttgggccttcctgccaattggtctccagcattctgccttttattttggtttattagtctgctttccatacctatcagcatagctaaggatttatctgatattgaaggtgataggaagtttggaatcattactttctctactaagttcggggcaaaaccgatcgcgtacatatgtcacgggcttatgcttttgaattacgtgagtgttatggccgcggccataatatggcctcaattcttcaactcctcagtaatactgttatcacatgccttcatggcgatctgggttttgtaccaagcgtggatactggagaaaagtaactatgcaacggaaacttgccagaaatattacatcttcttatggataatattctcccttgagcacgctttttacctattcatgtag

SEQ ID NO: 45 - DNA, 6268 bp, synthetic construct
  misc_feature 1..6268: note = Codon Optimized
  source 1..6268: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatggtattttcctcagtgtgtagttttccgtcctctcttggtacaaactttaagctggtgcctagatctaattttaaggcttcaagttcacattaccacgaaatcaacaatttcattaacaacaaacccattaaatttagttatttctcttcaaggttgtattgcagtgccaagccaatagtacacagagaaaacaagttcacaaaatcattctcactatcacacttacaacgtaaatcttctatcaaggcccatggagagatagaggctgatggaagtaacgggacttctgagttcaacgtaatgaagtccggaaatgctatctggagatttgtgaggccgtatgccgctaaaggtgtcctgtttaactccgcggcaatgttcgctaaggaacttgttggaaatctgaacttatttagctggccgttgatgttcaagatcctttcatttactcttgtcattctgtgtatctttgtatctacatcaggcataaatcagatatatgatctagacatcgatagactgaacaaaccgaacttgcccgtggcaagcggggaaattagcgtagaattggcatggttacttactatagtatgtacgattagtggacttaccttaaccattataactaatagtggccccttttttccgttcctttactcagcctccatattctttggtttcctatactccgcccccccgttccgttggaagaaaaacccctttaccgcctgcttttgcaatgtgatgttatacgtgggaaccagtgttggggtttattatgcctgcaaagccagtttgggccttcctgccaattggtctccagcattctgccttttattttggtttattagtctgctttccatacctatcagcatagctaaggatttatctgatattgaaggtgataggaagtttggaatcattactttctctactaagttcggggcaaaaccgatcgcgtacatatgtcacgggcttatgcttttgaattacgtgagtgttatggccgcggccataatatggcctcaattcttcaactcctcagtaatactgttatcacatgccttcatggcgatctgggttttgtaccaagcgtggatactggagaaaagtaactatgcaacggaaacttgccagaaatattacatcttcttatggataatattctcccttgagcacgctttttacctattcatgtaggatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 46 - DNA, 6268 bp, synthetic construct; identical to SEQ ID NO: 45
  misc_feature 1..6268: note = Codon Optimized
  source 1..6268: mol_type = other DNA

SEQ ID NO: 47 - DNA, 6256 bp, synthetic construct
  misc_feature 1..6256: note = Codon Optimized
  source 1..6256: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatgagattcccatctattttcactgctgttttgttcgctgcttcttctgctttggctgctccagttgcttcaagttcacattaccacgaaatcaacaatttcattaacaacaaacccattaaatttagttatttctcttcaaggttgtattgcagtgccaagccaatagtacacagagaaaacaagttcacaaaatcattctcactatcacacttacaacgtaaatcttctatcaaggcccatggagagatagaggctgatggaagtaacgggacttctgagttcaacgtaatgaagtccggaaatgctatctggagatttgtgaggccgtatgccgctaaaggtgtcctgtttaactccgcggcaatgttcgctaaggaacttgttggaaatctgaacttatttagctggccgttgatgttcaagatcctttcatttactcttgtcattctgtgtatctttgtatctacatcaggcataaatcagatatatgatctagacatcgatagactgaacaaaccgaacttgcccgtggcaagcggggaaattagcgtagaattggcatggttacttactatagtatgtacgattagtggacttaccttaaccattataactaatagtggccccttttttccgttcctttactcagcctccatattctttggtttcctatactccgcccccccgttccgttggaagaaaaacccctttaccgcctgcttttgcaatgtgatgttatacgtgggaaccagtgttggggtttattatgcctgcaaagccagtttgggccttcctgccaattggtctccagcattctgccttttattttggtttattagtctgctttccatacctatcagcatagctaaggatttatctgatattgaaggtgataggaagtttggaatcattactttctctactaagttcggggcaaaaccgatcgcgtacatatgtcacgggcttatgcttttgaattacgtgagtgttatggccgcggccataatatggcctcaattcttcaactcctcagtaatactgttatcacatgccttcatggcgatctgggttttgtaccaagcgtggatactggagaaaagtaactatgcaacggaaacttgccagaaatattacatcttcttatggataatattctcccttgagcacgctttttacctattcatgtaggatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 48 - DNA, 648 bp, synthetic construct
  misc_feature 1..648: note = Codon Optimized
  source 1..648: mol_type = other DNA
atgagcatcgaaatggcctgggtcctgaccatattctgtgctatcagtgggttaatacttacaatcactatgaacagcggccctctatttccattcttgtactgtggatctatatttgttgctggctttctatatagtgctccgcccttcagatttaagaataaccacttcactgccctgctgtgtaattacgtaatgtttgtcagcacaacccttcagatatactgcgcatacaaggcgggccttggccttccactgaattggagccccgcgttctgcctattagtgtggttcttgtcattaatcgctgtcactatatgtattggcaaagatttgtcagacattgaaggcgatagaaagttcggcgtaacaaccttcccgacagaatacggggcaaagcccatagcgctaatttgccacggcctgattctattagactacgtgggtctgatggcagccgccataatctggccgcagttattcaactctaagctaatcctactgtctcatgcgtttatggccgtgtgggtcgtttatcaggcttggattttggaaaagagcaattatacgaccgaggcatgtcaaaagtactatatgtacttatggacgatctattctgtcgagcacatcttatatctgttcatgtag

SEQ ID NO: 49 - DNA, 423 bp, synthetic construct
  misc_feature 1..423: note = Codon Optimized
  source 1..423: mol_type = other DNA
atggcatacaaggcgggccttggccttccactgaattggagccccgcgttctgcctattagtgtggttcttgtcattaatcgctgtcactatatgtattggcaaagatttgtcagacattgaaggcgatagaaagttcggcgtaacaaccttcccgacagaatacggggcaaagcccatagcgctaatttgccacggcctgattctattagactacgtgggtctgatggcagccgccataatctggccgcagttattcaactctaagctaatcctactgtctcatgcgtttatggccgtgtgggtcgtttatcaggcttggattttggaaaagagcaattatacgaccgaggcatgtcaaaagtactatatgtacttatggacgatctattctgtcgagcacatcttatatctgttcatgtag

SEQ ID NO: 50 - DNA, 485 bp, synthetic construct
  misc_feature 1..485: note = Codon Optimized
  source 1..485: mol_type = other DNA
atgagattcccatctattttcactgctgttttgttcgctgcttcttctgctttggctgctccagttcatacaaggcgggccttggccttccactgaattggagccccgcgttctgcctattagtgtggttcttgtcattaatcgctgtcactatatgtattggcaaagatttgtcagacattgaaggcgatagaaagttcggcgtaacaaccttcccgacagaatacggggcaaagcccatagcgctaatttgccacggcctgattctattagactacgtgggtctgatggcagccgccataatctggccgcagttattcaactctaagctaatcctactgtctcatgcgtttatggccgtgtgggtcgtttatcaggcttggattttggaaaagagcaattatacgaccgaggcatgtcaaaagtactatatgtacttatggacgatctattctgtcgagcacatcttatatctgttcatgtag

SEQ ID NO: 51 - DNA, 5713 bp, synthetic construct
  misc_feature 1..5713: note = Codon Optimized
  source 1..5713: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatgagcatcgaaatggcctgggtcctgaccatattctgtgctatcagtgggttaatacttacaatcactatgaacagcggccctctatttccattcttgtactgtggatctatatttgttgctggctttctatatagtgctccgcccttcagatttaagaataaccacttcactgccctgctgtgtaattacgtaatgtttgtcagcacaacccttcagatatactgcgcatacaaggcgggccttggccttccactgaattggagccccgcgttctgcctattagtgtggttcttgtcattaatcgctgtcactatatgtattggcaaagatttgtcagacattgaaggcgatagaaagttcggcgtaacaaccttcccgacagaatacggggcaaagcccatagcgctaatttgccacggcctgattctattagactacgtgggtctgatggcagccgccataatctggccgcagttattcaactctaagctaatcctactgtctcatgcgtttatggccgtgtgggtcgtttatcaggcttggattttggaaaagagcaattatacgaccgaggcatgtcaaaagtactatatgtacttatggacgatctattctgtcgagcacatcttatatctgttcatgtaggatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 52 - DNA, 5488 bp, synthetic construct
  misc_feature 1..5488: note = Codon Optimized
  source 1..5488: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatggcatacaaggcgggccttggccttccactgaattggagccccgcgttctgcctattagtgtggttcttgtcattaatcgctgtcactatatgtattggcaaagatttgtcagacattgaaggcgatagaaagttcggcgtaacaaccttcccgacagaatacggggcaaagcccatagcgctaatttgccacggcctgattctattagactacgtgggtctgatggcagccgccataatctggccgcagttattcaactctaagctaatcctactgtctcatgcgtttatggccgtgtgggtcgtttatcaggcttggattttggaaaagagcaattatacgaccgaggcatgtcaaaagtactatatgtacttatggacgatctattctgtcgagcacatcttatatctgttcatgtaggatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 53 - DNA, 5550 bp, synthetic construct
  misc_feature 1..5550: note = Codon Optimized
  source 1..5550: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatgagattcccatctattttcactgctgttttgttcgctgcttcttctgctttggctgctccagttcatacaaggcgggccttggccttccactgaattggagccccgcgttctgcctattagtgtggttcttgtcattaatcgctgtcactatatgtattggcaaagatttgtcagacattgaaggcgatagaaagttcggcgtaacaaccttcccgacagaatacggggcaaagcccatagcgctaatttgccacggcctgattctattagactacgtgggtctgatggcagccgccataatctggccgcagttattcaactctaagctaatcctactgtctcatgcgtttatggccgtgtgggtcgtttatcaggcttggattttggaaaagagcaattatacgaccgaggcatgtcaaaagtactatatgtacttatggacgatctattctgtcgagcacatcttatatctgttcatgtaggatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 54 - DNA, 1155 bp, synthetic construct
  misc_feature 1..1155: note = Codon Optimized
  source 1..1155: mol_type = other DNA
atggaattatcattatctctgggcgggccgacgatattccccagatatagagcaagctatacttccactaaactgaccactcatttctctaattttccgtccaaattcagcacaaaaaatttccaccagacgctatctttctacggaccaacgagaggcagcaaatcattgttgaatacccatcagtggaggaactccataagagcctgcgccgaggcgggggctgccgggtcaaacccggtgctaaacaaggtctctgactttagggacgcatgctggcgtttcttaaggccgcatactataagggggaccaccctgggtagtatagccctagtagcaagggcgcttatagaaaatcccaacttaatcaagtggagtcttttactgaaggctttctctggcttactagccttaatttgcgggaacggctacattgtaggaatcaaccaaatctatgacataggtattgataaggtcaacaaaccgtaccttcccatagccgcgggtgatttgtcagtccagagtgcttggtaccttgtaatattgtttgccgttgcgggtctactaactgttggcttcaattttgggccgttcattacgtctctatactgcttaggacttgttctggggacaatatatagcgttccaccctttaggatgaaaagatttccggttgcagcattcttaatcattgcgaccgtgaggggttttctattaaactttggtgtatactacgccacaagagctgcattgggcttaacctttgaatggagtagtgcggttgcgttcattacgaccttcgttacattattcgctttggtgatagctataacgaaagatctgccagacgtggagggagaccgtaaatttcagatcagcacattcgcaacaaagcttggtgtgagaaacatcgcgtatcttggatctggactattattattaaactacattggggcaattgcggcggccatttatatgcctcaggcttttaagagaaatttaatgcttcctatccacaccatcttggcgttgagccttgtcttccaggcctgggtcttggaacaagcgaattacaccaaggaggctattgctgggttctatagattcatttggaatttgttctatgtagaatacattatattcccctttatatag

SEQ ID NO: 55 - DNA, 933 bp, synthetic construct
  misc_feature 1..933: note = Codon Optimized
  source 1..933: mol_type = other DNA
atggccgggtcaaacccggtgctaaacaaggtctctgactttagggacgcatgctggcgtttcttaaggccgcatactataagggggaccaccctgggtagtatagccctagtagcaagggcgcttatagaaaatcccaacttaatcaagtggagtcttttactgaaggctttctctggcttactagccttaatttgcgggaacggctacattgtaggaatcaaccaaatctatgacataggtattgataaggtcaacaaaccgtaccttcccatagccgcgggtgatttgtcagtccagagtgcttggtaccttgtaatattgtttgccgttgcgggtctactaactgttggcttcaattttgggccgttcattacgtctctatactgcttaggacttgttctggggacaatatatagcgttccaccctttaggatgaaaagatttccggttgcagcattcttaatcattgcgaccgtgaggggttttctattaaactttggtgtatactacgccacaagagctgcattgggcttaacctttgaatggagtagtgcggttgcgttcattacgaccttcgttacattattcgctttggtgatagctataacgaaagatctgccagacgtggagggagaccgtaaatttcagatcagcacattcgcaacaaagcttggtgtgagaaacatcgcgtatcttggatctggactattattattaaactacattggggcaattgcggcggccatttatatgcctcaggcttttaagagaaatttaatgcttcctatccacaccatcttggcgttgagccttgtcttccaggcctgggtcttggaacaagcgaattacaccaaggaggctattgctgggttctatagattcatttggaatttgttctatgtagaatacattatattcccctttatatag

SEQ ID NO: 56 - DNA, 996 bp, synthetic construct
  misc_feature 1..996: note = Codon Optimized
  source 1..996: mol_type = other DNA
atgagattcccatctattttcactgctgttttgttcgctgcttcttctgctttggctgctccagttgccgggtcaaacccggtgctaaacaaggtctctgactttagggacgcatgctggcgtttcttaaggccgcatactataagggggaccaccctgggtagtatagccctagtagcaagggcgcttatagaaaatcccaacttaatcaagtggagtcttttactgaaggctttctctggcttactagccttaatttgcgggaacggctacattgtaggaatcaaccaaatctatgacataggtattgataaggtcaacaaaccgtaccttcccatagccgcgggtgatttgtcagtccagagtgcttggtaccttgtaatattgtttgccgttgcgggtctactaactgttggcttcaattttgggccgttcattacgtctctatactgcttaggacttgttctggggacaatatatagcgttccaccctttaggatgaaaagatttccggttgcagcattcttaatcattgcgaccgtgaggggttttctattaaactttggtgtatactacgccacaagagctgcattgggcttaacctttgaatggagtagtgcggttgcgttcattacgaccttcgttacattattcgctttggtgatagctataacgaaagatctgccagacgtggagggagaccgtaaatttcagatcagcacattcgcaacaaagcttggtgtgagaaacatcgcgtatcttggatctggactattattattaaactacattggggcaattgcggcggccatttatatgcctcaggcttttaagagaaatttaatgcttcctatccacaccatcttggcgttgagccttgtcttccaggcctgggtcttggaacaagcgaattacaccaaggaggctattgctgggttctatagattcatttggaatttgttctatgtagaatacattatattcccctttatatag

SEQ ID NO: 57 - DNA, 6220 bp, synthetic construct
  misc_feature 1..6220: note = Codon Optimized
  source 1..6220: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatggaattatcattatctctgggcgggccgacgatattccccagatatagagcaagctatacttccactaaactgaccactcatttctctaattttccgtccaaattcagcacaaaaaatttccaccagacgctatctttctacggaccaacgagaggcagcaaatcattgttgaatacccatcagtggaggaactccataagagcctgcgccgaggcgggggctgccgggtcaaacccggtgctaaacaaggtctctgactttagggacgcatgctggcgtttcttaaggccgcatactataagggggaccaccctgggtagtatagccctagtagcaagggcgcttatagaaaatcccaacttaatcaagtggagtcttttactgaaggctttctctggcttactagccttaatttgcgggaacggctacattgtaggaatcaaccaaatctatgacataggtattgataaggtcaacaaaccgtaccttcccatagccgcgggtgatttgtcagtccagagtgcttggtaccttgtaatattgtttgccgttgcgggtctactaactgttggcttcaattttgggccgttcattacgtctctatactgcttaggacttgttctggggacaatatatagcgttccaccctttaggatgaaaagatttccggttgcagcattcttaatcattgcgaccgtgaggggttttctattaaactttggtgtatactacgccacaagagctgcattgggcttaacctttgaatggagtagtgcggttgcgttcattacgaccttcgttacattattcgctttggtgatagctataacgaaagatctgccagacgtggagggagaccgtaaatttcagatcagcacattcgcaacaaagcttggtgtgagaaacatcgcgtatcttggatctggactattattattaaactacattggggcaattgcggcggccatttatatgcctcaggcttttaagagaaatttaatgcttcctatccacaccatcttggcgttgagccttgtcttccaggcctgggtcttggaacaagcgaattacaccaaggaggctattgctgggttctatagattcatttggaatttgttctatgtagaatacattatattcccctttatataggatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 58 - DNA, 6220 bp, synthetic construct; identical to SEQ ID NO: 57
  misc_feature 1..6220: note = Codon Optimized
  source 1..6220: mol_type = other DNA

SEQ ID NO: 59 - DNA, 6061 bp, synthetic construct
  misc_feature 1..6061: note = Codon Optimized
  source 1..6061: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccatcatgagattcccatctattttcactgctgttttgttcgctgcttcttctgctttggctgctccagttgccgggtcaaacccggtgctaaacaaggtctctgactttagggacgcatgctggcgtttcttaaggccgcatactataagggggaccaccctgggtagtatagccctagtagcaagggcgcttatagaaaatcccaacttaatcaagtggagtcttttactgaaggctttctctggcttactagccttaatttgcgggaacggctacattgtaggaatcaaccaaatctatgacataggtattgataaggtcaacaaaccgtaccttcccatagccgcgggtgatttgtcagtccagagtgcttggtaccttgtaatattgtttgccgttgcgggtctactaactgttggcttcaattttgggccgttcattacgtctctatactgcttaggacttgttctggggacaatatatagcgttccaccctttaggatgaaaagatttccggttgcagcattcttaatcattgcgaccgtgaggggttttctattaaactttggtgtatactacgccacaagagctgcattgggcttaacctttgaatggagtagtgcggttgcgttcattacgaccttcgttacattattcgctttggtgatagctataacgaaagatctgccagacgtggagggagaccgtaaatttcagatcagcacattcgcaacaaagcttggtgtgagaaacatcgcgtatcttggatctggactattattattaaactacattggggcaattgcggcggccatttatatgcctcaggcttttaagagaaatttaatgcttcctatccacaccatcttggcgttgagccttgtcttccaggcctgggtcttggaacaagcgaattacaccaaggaggctattgctgggttctatagattcatttggaatttgttctatgtagaatacattatattcccctttatataggatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 60 - DNA, 1161 bp, synthetic construct
  misc_feature 1..1161: note = Codon Optimized
  source 1..1161: mol_type = other DNA
atgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgattctgatttggaagcttctttgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattggatttgaaaaaagaaaaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttgttagatctgttccaattaaatat

SEQ ID NO: 61 - DNA, 1155 bp, synthetic construct
  misc_feature 1..1155: note = Codon Optimized
  source 1..1155: mol_type = other DNA
atgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgattctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttgttagatctgttccaattaaatat

SEQ ID NO: 62 - DNA, 1155 bp, synthetic construct
  misc_feature 1..1155: note = Codon Optimized
  source 1..1155: mol_type = other DNA
atgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgattctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaaaaagaaaaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaactgttgttttgagatctgttccaattaattat

SEQ ID NO: 63 - DNA, 1155 bp, synthetic construct
  misc_feature 1..1155: note = Codon Optimized
  source 1..1155: mol_type = other DNA
atgaatcatttgagagctgaaggtccagcttctgttttggctattggtactgctaatccagaaaatattttgttgcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgaatctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttgttagatctgttccaattaaatat

SEQ ID NO: 64 - DNA, 1155 bp, synthetic construct
  misc_feature 1..1155: note = Codon Optimized
  source 1..1155: mol_type = other DNA
atgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaatatttttttgaatgaagaacatttgaaacaaaatccaaaattggttgaacatgatgttcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgattctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttgttagatctgttccaattaaatat

SEQ ID NO: 65 - DNA, 1155 bp, synthetic construct
  misc_feature 1..1155: note = Codon Optimized
  source 1..1155: mol_type = other DNA
atgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatatgactgcttgtttgtttagaggtccatctgattctaatttggaattgttggttggtcaagctatttttggtgatggtgctgctgctgttattgttggtgctgaaccagatgaatctgttggtgaaagaccaatttttgaattggtttctactggtcaaacttttttgccaaattctgaaggtactattggtggtcatattagagaagctggtttgatgtttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttttgagatctgttccaattaattat

SEQ ID NO: 66 - DNA, 1173 bp, synthetic construct
  misc_feature 1..1173: note = Codon Optimized
  source 1..1173: mol_type = other DNA
atggaagaaattaaaggtgttttgaaagctaaagatgttggttgtgttgctactattttggctattggtactgctaatccattgaattgtgttaatcaagatgaatttttgcattcttattttaaattgactaataatcataataatacttcttttaaagaattgtttactagaatttgtaataattctatgattaaaaatagatatatgcatttgactgaagatattttgaaagaaaatccaaatttgtgtgattatgctgctcaatctttgaatactagacaagatattaaaattaaagaaattccaaaattggctgaaagagctgctatggttgctattaaagaatggggtaaaccaatttctaatttgactcatattatttttcattcttctactggtgctgctgatatgccaggtgctgattatcaattggttaaatctttgggtttgaatagatctattaaaagaattatgttgtataatttgggttgttttgctggtggtactgttttgagagttgctaaagatttggttgaaaataatttgggtgcttctgttttggctgtttgtgctgaaattacttctgctgatgctacttttggtagattgtctgaagatgataaaggtagattggttggtcatgctatttttggtgatggtgctgctgctttggttattggtaatgctgatgatccagaaaataaaggtttgtttcaaattgtttctacttctcaaactattttgccaaattctgaaggttgtattgaaggtcatattagagaagatggtgttacttttactttgtctccaagagttccaaaattgattggtgataatattgaaacttgtttgatggaagcttttactccatttaaaatttctgattggaattctttgttttgggttgttcatccaggtggtgctgctattttgagagaagttgaatctagagttggtttggaacaagaaaaattgagagcttcttggcatgttttgagagaatatggtaatatttcttctgcttctgttttgtttattttggatgaaatgagaaataaatctttggaagaaggtagaaaaactactggtgaaggtaaaaattggggtgttttgtttggttttggtccaggtttgactgttgaaactgttgttttgcattctattccaatt

SEQ ID NO: 67 - DNA, 1155 bp, synthetic construct; identical to SEQ ID NO: 61
  misc_feature 1..1155: note = Codon Optimized
  source 1..1155: mol_type = other DNA

SEQ ID NO: 68 - DNA, 1200 bp, synthetic construct
  misc_feature 1..1200: note = Codon Optimized
  source 1..1200: mol_type = other DNA
atgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgattctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagaggtagatggagaaaaggtaatttgccattggaaatggatttgtctggtgttttttttttgggtttggatcaagtt

SEQ ID NO: 69 - DNA, 1167 bp, synthetic construct
  misc_feature 1..1167: note = Codon Optimized
  source 1..1167: mol_type = other DNA
atggttactgttgaagaatttagaaaagctcaaagagctgaaggtccagctactattatggctattggtactgctactccagctaattgtgttttgcaatctgaatatccagattattattttagaattactaattctgaacataaaactgaattgaaagaaaaatttaaaagaatgtgtgataaatctatgattagaaaaagatatatgcatttgactgaagaaattttgaaagaaaatccaaatttgtgtgcttatgaagctccatctttggatgctagacaagatatggttgttgttgaagttccaaaattgggtaaagaagctgctactaaagctattaaagaatggggtcaaccaaaatctaaaattactcatttggttttttgtactacttctggtgttgatatgccaggtgctgattatcaattgactaaattgttgggtttgagaccatctgttaaaagattgatgatgtatcaacaaggttgttttgctggtggtactgttttgagattggctaaagatttggctgaaaataataaaggtgctagagttttggttgtttgttctgaaattactgctgttacttttagaggtccaaatgatactcatttggattctttggttggtcaagctttgtttggtgatggttctgctgctttgattgttggttctgatccaattccagaagttgaaaaaccaatttttgaattggtttctgctgctcaaactattttgccagattctgatggtgctattgatggtcatttgagagaagttggtttgacttttcatttgttgaaagatgttccaggtttgatttctaaaaatattgaaaaatctttgaatgaagcttttaaaccattgggtatttctgattggaattctttgttttggattgctcatccaggtggtccagctattttggatcaagttgaatctaaattggctttgaaaactgaaaaattgagagctactagacatgttttgtctgaatatggtaatatgtcttctgcttgtgttttgtttattttggatgaaatgagaagaaaatgtgttgaagatggtttgaatactactggtgaaggtttggaatggggtgttttgtttggttttggtccaggtttgactgttgaaactgttgttttgcattctgttgctatt

SEQ ID NO: 70 - DNA, 573 bp, synthetic construct
  misc_feature 1..573: note = Codon Optimized
  source 1..573: mol_type = other DNA
atgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggctgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggctttttttttt

SEQ ID NO: 71 - DNA, 597 bp, synthetic construct
  misc_feature 1..597: note = Codon Optimized
  source 1..597: mol_type = other DNA
atggcttgtttgtttagaggtccatctgaatctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttgttagatctgttccaattaaatat

SEQ ID NO: 72 - DNA, 936 bp, synthetic construct
  misc_feature 1..936: note = Codon Optimized
  source 1..936: mol_type = other DNA
atgaatttggaaaatattgataaagttaattctccaggtactgaagataaagattttgattctagagcttctggttctaaaactaatggttgtgaatcttctgataatgaagttgaatcttctattaatgctaatccaaattctatttctggttcttcttctggttttggtaatggtaaaagagaaggtgttaaaagagctgctccaggtgatattgctccaacttctagacattatagatctttgtctatggattcttatatgggttctttgcaatttgatgatgaatctttgaaattgttgccattgggtactggtgttggtttgcaatctccaaattctttggctgatggtaattctactaaatttggtatggaatttccaaatggtgaatttaatgctgttgaattgaaaaaaattatggaatctgaaaaattgactgaaattgctttgtctgatccaaaaagagctaaaagaattttggctaatagacaatctgctgctagatctaaagaaagaagatctagatatatttctgaattggaacataaagttcaaactttgcaaactgaagctactactttgtctgctcaagttactaaattgcaaagagattctgttggtttgacttctcaaaattctgaattgaaatttagagttcaagctatggaacaacaagctcaattgaaagatgctttgaatgatgctttgagagctgaagttcaaagattgaaattgactgctgctgaattgtctggtgaagctcatttgtctaattgtatggctcaacaattgtctattaatcaacaaatgtatcaaatgcaacatagacaaactgttcaattgaatttgtatcaaatgcaacaacaacaacaacataatgaaatgtcttctcaaccatgttctggtgaagttactgaacatgaatcttctaaa

SEQ ID NO: 73 - DNA, 10349 bp, synthetic construct
  misc_feature 1..10349: note = Codon Optimized
  source 1..10349: mol_type = other DNA
attcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtcgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccattcttttaatcgtggatccttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactataatgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgattctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttgttagatctgttccaattaaatatgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgatgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttc

SEQ ID NO: 74 - DNA, 10349 bp, synthetic construct
  misc_feature 1..10349: note = Codon Optimized
  source 1..10349: mol_type = other DNA
attcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtcgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccattcttttaatcgtggatccttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactataatgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgattctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaaaaagaaaaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaactgttgttttgagatctgttccaattaattatgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgatgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttc

SEQ ID NO: 75 - DNA, 10349 bp, synthetic construct
  misc_feature 1..10349: note = Codon Optimized
  source 1..10349: mol_type = other DNA
attcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtcgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccattcttttaatcgtggatccttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactataatgaatcatttgagagctgaaggtccagcttctgttttggctattggtactgctaatccagaaaatattttgttgcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgaatctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttgttagatctgttccaattaaatatgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgatgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttc

SEQ ID NO: 76 - DNA, 10349 bp, synthetic construct
  misc_feature 1..10349: note = Codon Optimized
  source 1..10349: mol_type = other DNA
attcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtcgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccattcttttaatcgtggatccttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactataatgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaatatttttttgaatgaagaacatttgaaacaaaatccaaaattggttgaacatgatgttcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgattctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttgttagatctgttccaattaaatatgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgatgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttc

SEQ ID NO: 77 - DNA, 10349 bp, synthetic construct
  misc_feature 1..10349: note = Codon Optimized
  source 1..10349: mol_type = other DNA
attcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtcgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccattcttttaatcgtggatccttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactataatgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatatgactgcttgtttgtttagaggtccatctgattctaatttggaattgttggttggtcaagctatttttggtgatggtgctgctgctgttattgttggtgctgaaccagatgaatctgttggtgaaagaccaatttttgaattggtttctactggtcaaacttttttgccaaattctgaaggtactattggtggtcatattagagaagctggtttgatgtttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttttgagatctgttccaattaattatgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgatgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttc

SEQ ID NO: 78 - DNA, 10367 bp, synthetic construct
  misc_feature 1..10367: note = Codon Optimized
  source 1..10367: mol_type = other DNA
attcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtcgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccattcttttaatcgtggatccttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactataatggaagaaattaaaggtgttttgaaagctaaagatgttggttgtgttgctactattttggctattggtactgctaatccattgaattgtgttaatcaagatgaatttttgcattcttattttaaattgactaataatcataataatacttcttttaaagaattgtttactagaatttgtaataattctatgattaaaaatagatatatgcatttgactgaagatattttgaaagaaaatccaaatttgtgtgattatgctgctcaatctttgaatactagacaagatattaaaattaaagaaattccaaaattggctgaaagagctgctatggttgctattaaagaatggggtaaaccaatttctaatttgactcatattatttttcattcttctactggtgctgctgatatgccaggtgctgattatcaattggttaaatctttgggtttgaatagatctattaaaagaattatgttgtataatttgggttgttttgctggtggtactgttttgagagttgctaaagatttggttgaaaataatttgggtgcttctgttttggctgtttgtgctgaaattacttctgctgatgctacttttggtagattgtctgaagatgataaaggtagattggttggtcatgctatttttggtgatggtgctgctgctttggttattggtaatgctgatgatccagaaaataaaggtttgtttcaaattgtttctacttctcaaactattttgccaaattctgaaggttgtattgaaggtcatattagagaagatggtgttacttttactttgtctccaagagttccaaaattgattggtgataatattgaaacttgtttgatggaagcttttactccatttaaaatttctgattggaattctttgttttgggttgttcatccaggtggtgctgctattttgagagaagttgaatctagagttggtttggaacaagaaaaattgagagcttcttggcatgttttgagagaatatggtaatatttcttctgcttctgttttgtttattttggatgaaatgagaaataaatctttggaagaaggtagaaaaactactggtgaaggtaaaaattggggtgttttgtttggttttggtccaggtttgactgttgaaactgttgttttgcattctattccaattgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgatgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttc

SEQ ID NO: 79 - DNA, 10349 bp, synthetic construct; identical to SEQ ID NO: 73
  misc_feature 1..10349: note = Codon Optimized
  source 1..10349: mol_type = other DNA

SEQ ID NO: 80 - DNA, 10394 bp, synthetic construct
  misc_feature 1..10394: note = Codon Optimized
  source 1..10394: mol_type = other DNA
attcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtcgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccattcttttaatcgtggatccttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactataatgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgattctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagaggtagatggagaaaaggtaatttgccattggaaatggatttgtctggtgttttttttttgggtttggatcaagttgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgatgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttc

SEQ ID NO: 81 - DNA, 10361 bp, synthetic construct
  misc_feature 1..10361: note = Codon Optimized
  source 1..10361: mol_type = other DNA
attcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtcgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccattcttttaatcgtggatccttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactataatggttactgttgaagaatttagaaaagctcaaagagctgaaggtccagctactattatggctattggtactgctactccagctaattgtgttttgcaatctgaatatccagattattattttagaattactaattctgaacataaaactgaattgaaagaaaaatttaaaagaatgtgtgataaatctatgattagaaaaagatatatgcatttgactgaagaaattttgaaagaaaatccaaatttgtgtgcttatgaagctccatctttggatgctagacaagatatggttgttgttgaagttccaaaattgggtaaagaagctgctactaaagctattaaagaatggggtcaaccaaaatctaaaattactcatttggttttttgtactacttctggtgttgatatgccaggtgctgattatcaattgactaaattgttgggtttgagaccatctgttaaaagattgatgatgtatcaacaaggttgttttgctggtggtactgttttgagattggctaaagatttggctgaaaataataaaggtgctagagttttggttgtttgttctgaaattactgctgttacttttagaggtccaaatgatactcatttggattctttggttggtcaagctttgtttggtgatggttctgctgctttgattgttggttctgatccaattccagaagttgaaaaaccaatttttgaattggtttctgctgctcaaactattttgccagattctgatggtgctattgatggtcatttgagagaagttggtttgacttttcatttgttgaaagatgttccaggtttgatttctaaaaatattgaaaaatctttgaatgaagcttttaaaccattgggtatttctgattggaattctttgttttggattgctcatccaggtggtccagctattttggatcaagttgaatctaaattggctttgaaaactgaaaaattgagagctactagacatgttttgtctgaatatggtaatatgtcttctgcttgtgttttgtttattttggatgaaatgagaagaaaatgtgttgaagatggtttgaatactactggtgaaggtttggaatggggtgttttgtttggttttggtccaggtttgactgttgaaactgttgttttgcattctgttgctattgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgatgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttc

SEQ ID NO: 82 - DNA, 9767 bp, synthetic construct
  misc_feature 1..9767: note = Codon Optimized
  source 1..9767: mol_type = other DNA
attcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtcgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccattcttttaatcgtggatccttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactataatgaatcatttgagagctgaaggtccagcttctgttttggctattggtactgctaatccagaaaatattttgattcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggctgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttttttttttgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgatgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttc

SEQ ID NO: 83 - DNA, 9791 bp, synthetic construct
  misc_feature 1..9791: note = Codon Optimized
  source 1..9791: mol_type = other DNA
attcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtcgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccattcttttaatcgtggatccttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactataatggcttgtttgtttagaggtccatctgaatctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttgttagatctgttccaattaaatatgaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgatgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttc

SEQ ID NO: 84 - DNA, 10130 bp, synthetic construct
  misc_feature 1..10130: note = Codon Optimized
  source 1..10130: mol_type = other DNA
attcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtcgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatcattcgaaatgactgaattgttgtctcaaaactcttctcatgatcttgtttgttgcagttctaggtaaggatgacaatgggacaactctagtaactttgaataatgggttcaatttcttttgcaaacccaagttaaaggataatctcaattggttcaaatcaatggttgtgtcgtttgaatccttcaatacgaaaaatatgaccaattgttctggaccaccacccaaaggtggaacaccaatagcagtggtttcaaaaactctgtcatctacttcattacagactctttcgatttcgatagaactaattttgataccaccgatgttcatagtgtcatcggctctaccgtgtgcatggtagtaaccgttagaggtcaattcgaaaatgtcaccatgtcttctcaatacttcaccattcaaggttggcatacccttgaaatagacatcgtgatgattaccgtttaacaatgtttttgaggcaccaaacataacaggacctaatgccaattcaccgatacctggcttatttttaggcattgggtaaccgttcttatctaatatgtacaaggtgcaacccatacattgggatgaaaaagaacttaaagattgagcttgcaaaaatgaaccagcagaaaaagcaccaccgatttctgtaccaccacacatttctataactggcttgtagttagctctacccattaaccacaaatattcgtctacattagaggcttcaccggatgaagaaaagcatcttatggtggaccaatcgtaacctgaaacacaatttgtggatttccatgatcttacaatagatggtacgacacccaacattgtgacctttgcatcttgaacaaatttagcgaaaccagagactaaaggactaccgttgtacaaggcaatagatgcaccatttaacaaactagcataaaccaaccaaggacccatcatccaacccaaattagttggccatactataacgtcaccttttctaatatccaaatgagaccaaccatcagcagcagccttcaatggggtggcttgtgtccaaggaattgcttttggttcacctgtagtaccactggagaataagatgttagtataagcatcaacaggttgttctctggcagtaaactcgcagtttttaaactccttggctctttctaaaaagtaatcccaagatatgtcaccatctctcaattctgcaccaatgttagaaccactacaagggataactattgccattggggatttagcttcaactactcttgaatacaatggtattctctttttacctctgatgatgtgatcttgtgtgaaaattgccttagctttggataatctcaatctagttgagatttcaggggcggaaaatgaatctgctatagagacaactacgtaaccagccaatactatggccaaatatataacaacagcatcaacatgcattggcatatcgatggctattgcacaacctttttctaaacccatttcttccaatgcataaccaaccaaccaaactctctttctcaattgatctaatgtcaacttattcaaaggcaagtcatcgttaccctcgtctctccaaacgatcatagtatcgttcaatttcttattggagtttacgttcaagcaatttttagctgagttcaagtaaccaccaggtaaccattcagaaccacctgggttgttgatgtcatctcttctcaagatacattctgggtccttagagaaactaattttcatttcatccatcaatactgttctccaatagacttcagggtttctaacagaaaattcttggaagtgagaaaaagaagaaattggatctttgtactttacacccaaaaattctttacctctcttttccaacaaagcacccaaattagttgacttgactttttcagggtctggaatccaagcaggtggggctggaccgaaatccttgtagcaaccataaaacaacatttggtgtaaggagaaaggcaaatctggtgacaagatatggttagcgatgttgatccaagtttgaggggttgcagcaccataattacaaacgatttctgccaatctaccatgtaatgtttctgctacttctgaggtgatacccaatgcgatgaaatctgaggcaacgactgaatccaaggacttatagtttttacccattcttttaatcgtggatccttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactataatgaatttggaaaatattgataaagttaattctccaggtactgaagataaagattttgattctagagcttctggttctaaaactaatggttgtgaatcttctgataatgaagttgaatcttctattaatgctaatccaaattctatttctggttcttcttctggttttggtaatggtaaaagagaaggtgttaaaagagctgctccaggtgatattgctccaacttctagacattatagatctttgtctatggattcttatatgggttctttgcaatttgatgatgaatctttgaaattgttgccattgggtactggtgttggtttgcaatctccaaattctttggctgatggtaattctactaaatttggtatggaatttccaaatggtgaatttaatgctgttgaattgaaaaaaattatggaatctgaaaaattgactgaaattgctttgtctgatccaaaaagagctaaaagaattttggctaatagacaatctgctgctagatctaaagaaagaagatctagatatatttctgaattggaacataaagttcaaactttgcaaactgaagctactactttgtctgctcaagttactaaattgcaaagagattctgttggtttgacttctcaaaattctgaattgaaatttagagttcaagctatggaacaacaagctcaattgaaagatgctttgaatgatgctttgagagctgaagttcaaagattgaaattgactgctgctgaattgtctggtgaagctcatttgtctaattgtatggctcaacaattgtctattaatcaacaaatgtatcaaatgcaacatagacaaactgttcaattgaatttgtatcaaatgcaacaacaacaacaacataatgaaatgtcttctcaaccatgttctggtgaagttactgaacatgaatcttctaaagaaggtagaggttccttgttaacttgtggtgacgttgaagaaaacccaggtcctatggccgtcaagcatttgatagtattgaagtttaaagatgaaatcacagaagctcaaaaggaagaatttttcaagacctacgttaatttggtcaacattatacctgctatgaaagatgtatactggggtaaagacgttacacaaaagaaagaagaaggttatacacacattgtcgaagtaaccttcgaatcagttgaaactatccaagattacatcattcatccagctcacgttggttttggtgacgtttacagatccttctgggaaaaattgttgatcttcgattacaccccaagaaagtgatgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttc

SEQ ID NO: 85 - DNA, 2274 bp, synthetic construct
  misc_feature 1..2274: note = Codon Optimized
  source 1..2274: mol_type = other DNA
atgtatatgtatcaagaagtttatttggttccaactttgtcttatttgtatttggttgttgttttgttgccatctatttttttttcttttagaagaatggcttttaaatctttggattctgttacttcttctgatattgctgctttgggtattgaaccacaattggctcattctttgcatggtagattggctgaaattgtttctaatcatggttctgctactccacatacttggagatgtatttcttctcatttgttgtctccagatttgccattttctttgcatcaaatgttgtattatggttgttataaagattttggtccagatccaccagcttggattccagatgctgaaaatgctatttctactaatgttggtaaattgttggaaaaaagaggtaaagaatttttgggtgttaaatataaagatccaatttctaatttttctgattttcaagaattttctgttactaatccagaagtttattggagaactattttggatgaaatgaatatttctttttctaaaccaccagaatgtattttgagagaaaatttttctagagatggtcaaattttgaatccaggtggtgaatggttgccaggtgcttttattaatccagctaaaaattgtttggatttgaattgtaaatctttggatgatactatgattttgtggagagatgaaggtaaagatgatttgccagttaataaaatgactttgaaagaattgagatctgaagtttggttggttgcttatgctttgaaagaattggaattggaaggtggttctgctattgctattgatatgccaatgaatgttcattctgttgttatttatttggctattgttttggctggttatgttgttgtttctattgctgattcttttgctgctccagaaatttctactagattgaaaatttctaaagctaaagctatttttactcaagatttgattgttagaggtgaaaaaactattccattgtattctagaattgttgaagctcaatctccattggctattgttattccatctaaaggtttttctgtttctgctcaattgagacatggtgatgtttcttggcatgattttttgaatagagctaataaatttaaaaattatgaatttgctgctgttgaacaaccaattgatgcttatactaatattttgttttcttctggtactactggtgaaccaaaagctattccatggactcaagctactccatttaaagctgctgctgatgcttggtgtcatatggatattcaaaaaggtgatgttgttgcttggccaactaatttgggttggatgatgggtccatggttggtttatgcttctttgttgaatggtgcttctattgctttgtataatggttctccattgggttctggttttgctaaatttgttcaagatgctaaagttactatgttgggtgttattccatctattgttagaacttggaaatctactaattgtgttgctggttatgattggtctactattagatgtttttcttctactggtgaagcttctaatattgatgaatatttgtggttgatgggtagagcttattataaaccagttattgaatattgtggtggtactgaaattggtggtggttttgttactggttctttgttgcaagctcaatctttggctgctttttctactccagctatgggttgttctttgtttattttgggttctgatggttatccaattccaaaacataaaccaggtattggtgaattggctttgggtccattgatgtttggtgcttctaaaactttgttgaatgctgatcattatgatgtttattttaaaagaatgccatctttgaatggtaaagttttgagaagacatggtgatatgtttgaattgacttctaaaggttattatcatgctcatggtagagctgatgatactatgaatttgggtggtattaaagtttcttctgttgaaattgaaagaatttgtaatgaagctgatgaaaaagttttggaaactgctgctattggtgttccaccattggctggtggtccagaacaattggttattgctgttgttttgaaaaattctgatagaactactgttgatttgaatcaattgagattgtcttttaattctgctgttcaaaaaaaattgaatccattgtttagagtttctagagttgttccattgtcttctttgccaagaactgctactaataaagttatgagaagaattttgagacaacaatttactcaattggataaatcttctaaaatttaa

SEQ ID NO: 86 - DNA, 2331 bp, synthetic construct
  misc_feature 1..2331: note = Codon Optimized
  source 1..2331: mol_type = other DNA
atggctttggaattgccacatttgttgccatataaattggttaaaggtcaaactttggttgctcaagctgctagagctgaattggcttcttcttcttcttcttctgttattttgaaatctaattttattaataataattatattaattattgtaataataataataataatgaaagaagattggttgttagaagagattgggaaactatggcttcttctccatctcattctagaaataataatgatattagaactattaatcatttgagacatgttgattctatggctactatgccatctggtgctggtaaaattccaagattgaatgctgttattttgggtgaagctttggctactgaagaaaatgatttggtttttccaactgatgaattttctcaacaagctcatgttccatctccacaaaaatatttggaaatgtataaaagatctattgaagatccagctggtttttggtctgaaattgcttctcaattttattggaaacaaaaatgggatgattctgtttattctgaaaatttggatgtttctaaaggtagagttaatattgaatggtttaaaggtggtattactaatatttgttataattgtttggataaaaatgttgaagctggtttgggtgataaaattgctttgtattgggaaggtaatgatactggttttgatgattctttgacttattctcaattgttgcataaagtttgtcaattggctaattatttgaaagatatgggtgttcaaaaaggtgatgctgttgttatttatttgccaatgttgttggaattgccaattactatgttggcttgtgctagaattggtgctgttcattctgttgtttttgctggtttttctgctgaatctttgtctcaaagaattattgattgtaaaccaaaagttgttattacttgtaatgctgttaaaagaggtccaaaaattattcatttgaaagatattgttgatgctgctttggttgaatctgctaaaactggtgttccaattgatacttgtttggtttatgaaaatcaattggctatgaaaagagatattactaaatggcaagatggtagagatatttggtggcaagatgttattccaaaatatccaactgaatgtgctgttgaatgggttgatgctgaagatccattgtttttgttgtatacttctggttctactggtaaaccaaaaggtgttttgcatactactggtggttatatggtttatactgctactacttttaaatatgcttttgattataaaccatctgatgtttattggtgtactgctgattgtggttggattactggtcattcttatgttacttatggtccattgttgaatggtgcttcttgtattgtttttgaaggtgctccaaattatccagattctggtagatgttgggatattgttgataaatataaagttactattttttatactgctccaactttggttagatctttgatgagagatggtgatgaatatgttactagatattctagaaaatctttgagaattttgggttctgttggtgaaccaattaatccatctgcttggagatggttttataatgttgttggtgattctagatgtccaatttctgatacttggtggcaaactgaaactggtggttttatgattactccattgccaggtgcttggccacaaaaaccaggttctgctacttttccattttttggtgttaaaccagttattgttgatgaaaaaggtgttgaaattgaaggtgaatgttctggttatttgtgtgttaaaggttcttggccaggtgcttttagaactttgtatggtgattatgaaagatatgaaactacttattttaaaccatttactggttattattttactggtgatggttgttctagagataaagatggttatcattggttgactggtagagttgatgatgttattaatgtttctggtcatagaattggtactgctgaagttgaatctgctttggtttctcatccaaaatgtgctgaagctgctgttgttggtattgaacatgaagttaaaggtcaagctatttatgcttttgttactttggttgaaggtgaaccatattctgaagaattgagaaaatctttgattttgtctgttagaaaacaaattggtgcttttgctgctccagaaagaattcattgggctccaggtttgccaaaaactagatctggtaaaattatgagaagaattttgagaaaaattgcttctggtcaattggatgaattgggtgatacttctactttggctgatccaaatgttgttgaacaattgatttctttgtctaattgttag

SEQ ID NO: 87 - DNA, 1662 bp, synthetic construct
  misc_feature 1..1662: note = Codon Optimized
  source 1..1662: mol_type = other DNA
atggaaaaaaaacaagataatcataataataataataataatggtgatgaacaagaagaatttatttttagatctaaattgccagatatttatattccaaatcatccaccattgcattcttattgttttgaaaatatttctcaatttaaagatagaccatgtttgattaatggtgctactggtgaaactattacttatgctgatgttgatttgacttctagaaaagttgctgctggtttggataaaattggtattaaacaaggtgatgttattatgttgttgttgagaaattgtccagaatttgtttattcttttttggctgcttctcatattggtgctgttgttactactgctaatccattttatactgctgctgaagttgctaaacaagctgctgcttctaatactaaattggttattactttgtctggttttattgataaagttagagattttactggtgatggtattaaagttgtttgtgttgatgctccaccagatgaatctgaatatttgcatttttctgttttgactcaagctgatgaatctgaaattccagatgttgaaattaaaccagatgatgttgttgctttgccatattcttctggtactactggtttgccaaaaggtgttatgttgactcatagagttatggttactggtgttgctcaacaagttgatggtgataatccaaattggcattttcatcaaaatgatgttattttgtgtgttttgccagtttttcatatttattgtttgaatgctattttgttgtgtggtttgagagttggtgcttctattttgattatggaaaaatttgaaatgaaaaaaatggttgaattgattgaaaaatttaaagttactattgctccagttgttccaccaattgttttgtctgttgttaaatttccagatttgcatagatatgatttgtcttctattagaactattatgtctggtggtgctccaatgggtaaagatttggaagaagctgttaaagaaaaatttccacatgttactttgggtcaaggttatggtatgactgaagctgaatgtttgtctttgtgtttgggttttgctaaagaaccatttccaactaaatttggtacttgtggtactgttgttagaaatgctgaaatgaaaattgttgatccaaatactggtgcttctttgccaagaaatcaatctggtgaaatttgtattagaggtaaacaaattatgaaaggttatattaatgattttgaagctactaaaggtactattgatgaagctggttggttgcatactggtgatattggttttgttgatgatgatgatgaattgtttattgttgatagattgaaagaattgattaaatataaaggttttcaagttgctccagctgaattggaatctttgttgattgctcatccaaatatttctgatgctgctgttgttccaatgaaagatgaagctgctggtgaagttccagttgcttttgttgttagatctaatggttctaaaatttctgaagaagatattaaacaatatatttctaaacaagttgttttttataaaagaattgctaaagttttttttattgaagaaattccaaaatctccagctggtaaaattttgagaaaatctttgagagctagattggttactgaacaagctatttaa

SEQ ID NO: 88 - DNA, 1719 bp, synthetic construct
  misc_feature 1..1719: note = Codon Optimized
  source 1..1719: mol_type = other DNA
atgatttctattgctccaccattgaaaactcaaaataaacaagaaatttctactattgatcataatgatcataatcaagaacatatttttaaatctaaattgccagaaattccaatttctaataatattccattgcattcttatatttttcaaaatttgccagaaaaatctaatagaccatgtttgattactggtactactactcaaactacttattcttattctcaaactcatcatattgctaaaaaaattgctaaaggtttgtctaaattgaatattaataaaaatgatgttattatgattttgttgccaaattgtccagaatttattttttctttttttggtgcttctatgattggtgctgctattactactgctaatccattttatacttctccagaaatttttaaacaattgcaaatttctaaagctaaattggttattactcaaactcaatttgtttctaaattgattgattttccagaaaaagttattggtagagattttactgttgttactgttgatggtgatgaaaatccatctccagaaaattgtttgccattttctattttgactggtgaagatgaaactgaagaaatttctattgatccaaatgatccaattgctattccattttcttctggtactactggtttgccaaaaggtgttgttttgactcataaaaatttgattacttctgttgctcaacaagttgatggtgataatccaaatatgtatttgagatctgatgatgttgttttgtgtgttttgccattgtttcatatttattctttgaattctgttttgttgtgtgctttgagagttggtgcttctgttttgttggttccaaaatttgaaattggtactttgttggaattgattcaaaaacatagagttactgttgctccagttgttccaccattggttttgggtttggctaaaaatccagttgtttctgaatttgatttgtcttctattagaatggttttgtctggtgctgctccattgggtatggaattggaagatgctttgagaagaagagttccacaagctgttattggtcaaggttatggtatgactgaagctggtccagttttgtctatgtgtttggcttttgctaaacaaccatttccaactaaatctggttcttgtggtactgttgttagaaatgctcaattgaaagttattgatccagaaactggtgcttctttgtcttataatcaaccaggtgaaatttgtattagaggtcatcaaattatgaaaggttatttggataattctgatgctactgctaatactattgatgttgatggttggttgcatactggtgatattggttatgttgatgatgatgatgaaatttttattgttgatagagttaaagaaattattaaatttaaaggttttcaagttccaccagctgaattggaagctttgttgatttctcatccatctattgctgatgctgctgttgttccacaaaaagatgaagttgctggtgaagttccagttgcttttgttgttaaatctaataataaagattttgatttgtctgaagatgctgttaaagaatttattgctaaacaagttgttttttataaaaaattgcataaagtttattttgttcattctattccaaaatctccatctggtaaaattttgagaaaagatttggttgctaaattggctttggcttctactttgattatttcttcttaa

SEQ ID NO: 89 - DNA, 2184 bp, synthetic construct
  misc_feature 1..2184: note = Condon Optimized
  source 1..2184: mol_type = other DNA
atgggtagaaaatctatttctgaagttggtgttgaagatttggttcaagctggtttgactactgaagaagctactggttttcaaagagttttgaaagattctttgtcttgtactaaaggttctgatccatctgaagtttggagacatttggttgctagaagagttttgaaaccatggcatccacatggtttgcatcaattggtttattattctgtttatgctcattgggatgtttcttctaaaggtccaccaccatattggtttccatctttgtatgaatctaaacatactaatatgggtggtattatggaaaaacatggttcttctttgttgggtccattgtataaagatccaattacttcttattctttgtttcaaaaattttctgctcaacatccagaagcttattggtctattgttttgaaagaattgtctgtttcttttcaagaagaaccaaaatgtattttggatagatctgatttgaaatctaaacatggtggttcttggttgccaggttctgttttgaatattgctgaatgttgtttgttgccaactgcttatccaagaaaagatgatgattctttggctattgtttggagagatgaaggttgtgatgattctggtattaatattattactttgaaacaattgagagaacaagttatttctgttgctaaagctttggatgctatgttttctaaaggtgatgctattgctattgatatgccaatgactgctaatgctgttattatttatttggctattattttgtctggtttggttgttgtttctattgctgattcttttgctccaaaagaaatttctattagattgagagtttctcaagctaaagctatttttactcaagattttattttgagaggttctagaaaatttccattgtattctagagttgttgaagctgctccagataaagttattgttttgccagctattggttctaatgttggtattcaattgagagaacaagatatgtcttggggtgattttttgtcttctgttggtactagatctagaaattattctccatgttatcaaccagttgatactttgattaatattttgttttcttctggtactactggtgaaccaaaagctattccatggactcaattgtctccaattagatgtgctgctgaatcttgggctcatatggatatgcaagttggtgatgttttttgttggccaactaatttgggttgggttatgggtccaattttgattttttcttcttttttgtctggtgctactttggctttgtatcatggttctccattgggttatggttttggtaaatttgttcaagatgctggtgttactaaattgggtactgttccatctttggttaaagcttggaaaaatactcaatgtatgaatggtttggattggactaaaattaaatgttttgcttctactggtgaaacttctaatgttgatgatgatttgtggttgtcttctagagcttattataaaccagttattgaatgttgtggtggtactgaattgtcttcttcttatattcaaggttctttgttgcaaccacaagcttttggtgctttttctactacttctatgactacttctttggttattttggatgaacatggtaatccatttccagatgatcaagcttgtattggtgaagttggtttgtttccattgtatttgggtgctactgatagattgttgaatgctgatcatgaagaagtttattttaaaggtatgccattgtataaaggtatgagattgagaagacatggtgatattattaaaagaactgttggtggttattttattgttcaaggtagagctgatgatactatgaatttgggtggtattaaaacttcttctgttgaaattgaaagagtttgtgatagagctgatgaatctattgttgaaactgctgctgtttctgtttctccagttgatggtggtccagaacaattggttatgtttgttgttttgaaaaatggttataattctgaagctgaaaatttgagaactaaattttctaaagctattcaatctaatttgaatccattgtttaaagttagatttgttaaaattgttccagaatttccaagaactgcttctaataaattgttgagaagagttttgagagatcaaattaaacatgaattgtctgctcattctagaatttaa

SEQ ID NO: 90 - DNA, 2172 bp, synthetic construct
  misc_feature 1..2172: note = Codon Optimized
  source 1..2172: mol_type = other DNA
atgtctatttctgaagttggtgttgaagatttggttcaagctggtttgactactgaagaagctactggttttcaaagagttttgaaagattctttgtcttgtactaaaggttctgatccatctgaagtttggagacatttggttgctagaagagctttgaaaccatggcatccacatggtttgcatcaattggtttattattctgtttatgctcattgggatgtttcttctaaaggtccaccaccatattggtttccatctttgtatgaatctaaacatactaatatgggtggtattatggaaaaacatggttcttctttgttgggtccattgtataaagatccaattacttcttattctttgtttcaaaaattttctgctcaacatccagaagcttattggtctattgttttgaaagaattgtctgtttcttttcaagaagaaccaaaatgtattttggatagatctgatttgaaatctaaacatggtggttcttggttgccaggttctgttttgaatgttgctgaatgttgtttgttgccaactgcttatccaagaaaagatgatgattctttggctattgtttggagagatgaaggttgtgatgattctggtattaatattattactttgaaacaattgagagaacaagttatttctgttgctaaagctttggatgctatgttttctaaaggtgatgctattgctattgatatgccaatgactgctaatgctgttattatttatttggctattattttgtctggtttggttgttgtttctattgctgattcttttgctccaaaagaaatttctattagattgagagtttctcaagctaaagctatttttactcaagattttattttgagaggttctagaaaatttccattgtattctagagttgttgaagctgctccagataaagttattgttttgccagctattggttctaatgttggtattcaattgagagaacaagatatgtcttggggtgattttttgtcttctgttggtactagatctagaaattattctccatgttatcaaccagttgatactttgattaatattttgttttcttctggtactactggtgaaccaaaagctattccatggactcaattgtctccaattagatgtgctgctgaatcttgggctcatatggatatgcaagttggtgatgttttttgttggccaactaatttgggttgggttatgggtccaattttgattttttcttcttttttgtctggtgctactttggctttgtatcatggttctccattgggttatggttttggtaaatttgttcaagatgctggtgttactaaattgggtactgttccatctttggttaaagcttggaaaaatactcaatgtatgaatggtttggattggactaaaattaaatgttttgcttctactggtgaaacttctaatgttgatgatgatttgtggttgtcttctagagcttattataaaccagttattgaatgttgtggtggtactgaattgtcttcttcttatattcaaggttctttgttgcaaccacaagcttttggtgctttttctactacttctatgactacttctttggttattttggatgaacatggtaatccatttccagatgatcaagcttgtattggtgaagttggtttgtttccattgtatttgggtgctactgatagattgttgaatgctgatcatgaagaagtttattttaaagaatgtcattatactaaagaatgtgcttctgaaacttggagatattatcaaagaactgttggtggttattttattgttcaaggtagagctgatgatactatgaatttgggtggtattaaaacttcttctgttgaaattgaaagagtttgtgatagagctgatgaatctattgttgaaactgctgctgtttctgtttctccagttgatggtggtccagaacaattggttatgtttgttgttttgaaaaatggttataattctgaagctgaaaatttgagaactaaattttctaaagctattcaatctaatttgaatccattgtttaaagttagatttgttaaaattgttccagaatttccaagaactgcttctaataaattgttgagaagagttttgagagatcaaattaaacatgaattgtctgctcattctagaatttaa

SEQ ID NO: 91 - DNA, 2148 bp, synthetic construct
  misc_feature 1..2148: note = Codon Optimized
  source 1..2148: mol_type = other DNA
atgggttctaaatctatttctgaagttggtgttgatgatttggttcaagctggtttgactactgaagaagctactggttttcaaagagttttgaaagattctttgtcttgtactaaaggttctgatccatctgaagtttggagacatttggttgctagaagagttttgaaaccatggtctgtttatgctcattgggatgtttcttctaaaggtccaccaccatattggtttccatctttgtatcattctaaagatactaatttgggtagattgatggaaaaacatggtccatctttgttgggtccattgtataaagatccaattacttcttattctttgtttcaaaaattttctgttgaacatccagaagtttattggtctattgctttgaaagaattgtctgtttcttttcaagaagaaccaaaatgtattttggataaatctgataaatctaaacatggtggttcttggttgccaggtgctgttttgaatattgctgaatgttgtttgttgccaacttcttatccaagaaaagatgatgattctttggctattgtttggagagatgaaggttctgatgattcttctgttaatttgattactttgaaacaattgagagaacaagttatttctgttgctaaatctttggatgctatgttttctaaaggtgatgctattgctatggatatgccaatgactgctaatgctgttattatttatttggctattattttgtctggtttggttgttgtttctattgctgattcttttgctccaaaagaaattgcttctagattgcatgtttctcaagctaaagctatttttactcaagattttattttgagaggtggtagaaaatttccattgtattctagagttgttgaagctgctccagatagagttattgttttgccagctactggttctaatattggtattcaattgagagaacaagatatgtcttggggtgattttttgtcttctgttggtactagatctagaaaatattctccatgttatcaaccagttgattctttgattaatattttgttttcttctggtactactggtgaaccaaaagctattccatggactcatttgtctccaattagatgttcttctgatttttgggcttatatggatattaaagttggtgatgttgtttgttggccaactaatttgggttgggctttgggtccatttattttgtttacttgttttttgtctggtgctgttttggctttgtatcatggttctccattgggtagaggttttggtaaatttgttcaagatgcttctgttactaaattgggtactgttccatctttggttaaaacttggaaaaatactcaatgtatgaaaggtttggattggactaaaattaaatcttttgcttctactggtgaaacttctaatgttgatgatgatttgtggttgtcttctcaagcttattataaaccagttattgaatgttgtggtggtactgaattggcttcttcttatattcaaggttctttgttgcaaccacaagcttttggtgcttttaatactgctactatgactacttcttttgttattattgatgaacatggtaatccatatccagatgatcaagcttgtactggtgaagttggtttgattccattgtatttgggtgcttctgatagattgttgaatgctgatcatgaagaagtttattttaaaggtatgccattgtataaaggtatgagattgagaagacatggtgatattattaatagaactgttggtggttattttattgttcaaggtagagctgatgatactatgaatttgggtggtattaaaacttcttcttttgaaattgaacatgtttgtgatagagctgatgattctattttggaaactgctgctgtttctgtttctccaattggtggtggtccagaacaattggttatgtttgttgttttgaaaaatggttatgatgctgaagctgaaaatttgagaactaaattttctaaagctattcaatctaatttgaatccattgtttaaagttactgctgttaaaattgttcatgaatttccaagaactatgtctaataaattgttgagaagagttttgagagatcaattgaatagagaattttctattcaatctaaaatttag

SEQ ID NO: 92 - DNA, 303 bp, synthetic construct
  misc_feature 1..303: note = Codon Optimized
  source 1..303: mol_type = other DNA
atggctgttaaacatttgattattttgaaatttaaagatgaaattactgaagctcaaaaagaagaattttttaaaacttatgttaatttggttaatattattccagctatgaaagatgtttattggggtaaagatgttactcaaaaaaataaagaagaaggttatactcatattgttgaagttacttttgaatctgttgaaactattcaagattatattattcatccagctcatgttggttttggtgatgtttatagatgtttttgggaaaaattgttgatttttgattatactccaagaaaa

SEQ ID NO: 93 - DNA, 330 bp, synthetic construct
  misc_feature 1..330: note = Codon Optimized
  source 1..330: mol_type = other DNA
atggaagaagctaaaggtgttgttaaacatgttttgttggctaaatttaaagaaggtacttctgatgatcaaattcaacaattgattaaaggttatgctaatttgttgaatttgattccatctatgaaatcttttcattggggtaaagatgtttcttttgaaaatttgcatcaaggttttactcatatttttgaatctacttttgaaaatactgaaggtgttgctgaatatgttgctcatccagctcatgttgaatttgctaatgtttttttgtctaatttggataaagttgttgtttttgattataaaccaactactgttttgttgcca

SEQ ID NO: 94 - DNA, 42 bp, synthetic construct
  misc_feature 1..42: note = Primer
  source 1..42: mol_type = other DNA
aggaaacgaagataaatctcgagtttatcattatcaatactg

SEQ ID NO: 95 - DNA, 37 bp, synthetic construct
  misc_feature 1..37: note = Primer
  source 1..37: mol_type = other DNA
ggaaaaatcagtcaaggcaaattaaagccttcgagcg

SEQ ID NO: 96 - DNA, 27 bp, synthetic construct
  misc_feature 1..27: note = Primer
  source 1..27: mol_type = other DNA
gatgggggatccactagttctagaatc

SEQ ID NO: 97 - DNA, 25 bp, synthetic construct
  misc_feature 1..25: note = Primer
  source 1..25: mol_type = other DNA
tgatgggctgcaggaattcgatatc

SEQ ID NO: 98 - DNA, 38 bp, synthetic construct
  misc_feature 1..38: note = Primer
  source 1..38: mol_type = other DNA
gaactagtggatcccccatcatgaaccatttgagagcc

SEQ ID NO: 99 - DNA, 32 bp, synthetic construct
  misc_feature 1..32: note = Primer
  source 1..32: mol_type = other DNA
tattttggctttaactttcttggggtgtaatc

SEQ ID NO: 100 - DNA, 43 bp, synthetic construct
  misc_feature 1..43: note = Primer
  source 1..43: mol_type = other DNA
agaaagttaaagccaaaataatgataacgagaataatatcaag

SEQ ID NO: 101 - DNA, 28 bp, synthetic construct
  misc_feature 1..28: note = Primer
  source 1..28: mol_type = other DNA
ataaacccatggcgcagacctgtgagag

SEQ ID NO: 102 - DNA, 28 bp, synthetic construct
  misc_feature 1..28: note = Primer
  source 1..28: mol_type = other DNA
ggtctgcgccatgggtttatcatccgtc

SEQ ID NO: 103 - DNA, 41 bp, synthetic construct
  misc_feature 1..41: note = Primer
  source 1..41: mol_type = other DNA
cgaattcctgcagcccatcagtgtctatgtctaggtaaagg

SEQ ID NO: 104 - DNA, 25 bp, synthetic construct
  misc_feature 1..25: note = Primer
  source 1..25: mol_type = other DNA
tgatgggctgcaggaattcgatatc

SEQ ID NO: 105 - DNA, 27 bp, synthetic construct
  misc_feature 1..27: note = Primer
  source 1..27: mol_type = other DNA
gatgggggatccactagttctagaatc

SEQ ID NO: 106 - DNA, 33 bp, synthetic construct
  misc_feature 1..33: note = Primer
  source 1..33: mol_type = other DNA
caccagaaccgaaggtagaggttctttgttaac

SEQ ID NO: 107 - DNA, 45 bp, synthetic construct
  misc_feature 1..45: note = Primer
  source 1..45: mol_type = other DNA
cgaattcctgcagcccatcactttgatctcttgtagaccttattc

SEQ ID NO: 108 - DNA, 42 bp, synthetic construct
  misc_feature 1..42: note = Primer
  source 1..42: mol_type = other DNA
gaactagtggatcccccatcatggtttccaatcacttgtttg

SEQ ID NO: 109 - DNA, 29 bp, synthetic construct
  misc_feature 1..29: note = Primer
  source 1..29: mol_type = other DNA
ctctaccttcggttctggtgtataagtcg

SEQ ID NO: 110 - DNA, 22 bp, synthetic construct
  misc_feature 1..22: note = Primer
  source 1..22: mol_type = other DNA
gatccactagttctagaatccg

SEQ ID NO: 111 - DNA, 35 bp, synthetic construct
  misc_feature 1..35: note = Primer
  source 1..35: mol_type = other DNA
tctagaactagtggatcatgaaccatttgagagcc

SEQ ID NO: 112 - DNA, 28 bp, synthetic construct
  misc_feature 1..28: note = Primer
  source 1..28: mol_type = other DNA
tcgttatcactttcttggggtgtaatcg

SEQ ID NO: 113 - DNA, 36 bp, synthetic construct
  misc_feature 1..36: note = Primer
  source 1..36: mol_type = other DNA
ccaagaaagtgataacgagaataatatcaagaatac

SEQ ID NO: 114 - DNA, 44 bp, synthetic construct
  misc_feature 1..44: note = Primer
  source 1..44: mol_type = other DNA
aggtcgacggtatcgttaaataaaaacgtataccaaatattcag

SEQ ID NO: 115 - DNA, 18 bp, synthetic construct
  misc_feature 1..18: note = Primer
  source 1..18: mol_type = other DNA
cgataccgtcgacctcga

SEQ ID NO: 116 - DNA, 32 bp, synthetic construct
  misc_feature 1..32: note = Primer
  source 1..32: mol_type = other DNA
ggttaaactagtatgggtaaaaactataagtc

SEQ ID NO: 117 - DNA, 33 bp, synthetic construct
  misc_feature 1..33: note = Primer
  source 1..33: mol_type = other DNA
gtgcccgtcgactcattcgaaatgactgaattg

SEQ ID NO: 118 - DNA, 3952 bp, synthetic construct
  misc_feature 1..3952: note = Codon Optimized
  source 1..3952: mol_type = other DNA
tcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgaaattgttaacatttccaggtcaaggtacttctatttctatttctatattgaaggctataataagaaacaagtcaagagaatttcaaacaatcttgagtcaaaatggtaaagaatctaacgatttgttgcaatacatcttccaaaatccatcttcacctggttcaatcgctgtttgttccaatttgttctatcaattgtaccaaatcttgtctaacccatcagatcctcaagaccaagcacctaaaaatatgacaaagatcgatagtcctgacaaaaaggataacgaacaatgttatttgttgggtcattctttgggtgaattaacttgcttgtccgttaatagtttattttctttgaaggacttatttgatattgcaaacttcagaaacaaattaatggtcacttcaacagaaaagtacttggttgcccacaacataaacagatccaataagttcgaaatgtgggctttatccagtccaagagcaaccgatttgcctcaagaagttcaaaagttgttaaattcaccaaacttgttatcttcatcccaaaacactatttcagtagccaatgctaactccgtcaaacaatgtgttgtaactggtttagtagatgacttggaatcattaagaacagaattgaacttaagattccctagattgagaatcacagaattgaccaatccatacaacattcctttccataattccactgtcttaagaccagttcaagaacctttgtacgactatatttgggatattttgaaaaagaatggtactcatactttgatggaattaaaccacccaatcattgccaatttggatggtaacatctcatactacatccatcacgctttggatagattcgttaagtgttcttcaagaactgtacaattcacaatgtgctatgacaccattaatagtggtactccagttgaaatcgataagtctatttgtttcggtcctggtaacgtaatatacaacttaatcagaagaaactgccctcaagttgataccatcgaatacacttctttggcaacaatcgatgcctaccataaagctgcagaagaaaataaggattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaa

SEQ ID NO: 119 - DNA, 3718 bp, synthetic construct
  misc_feature 1..3718: note = Codon Optimized
  source 1..3718: mol_type = other DNA
tcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgaaaaaggtttgtgtcatcggtgctggtactatgggttctggtattgctcaagcatttgctgcaaaaggtttcgaagttgtattgagagatataaaggacgaatttgttgatagaggtttagacttcatcaataagaacttgagtaaattggttaagaaaggtaaaatcgaagaagctactaaggttgaaatattgacaagaatatctggtactgtagatttgaacatggccgctgattgtgacttagtaattgaagcagccgtcgaaagaatggatattaagaaacaaatattcgctgatttggataatatttgcaagccagaaactatattggcatctaacacatcttcattatcaattaccgaagtcgcctccgctactaagacaaacgataaggttataggcatgcatttctttaacccagcccctgttatgaaattggttgaagtaataagaggtatcgcaacctcacaagaaactttcgatgcagttaaggaaacatccattgccattggtaaagatccagtcgaagttgcagaagcccctggtttcgtcgttaacagaatcttgataccaatgataaacgaagctgtaggtatattagctgaaggtatcgcatccgtcgaagatattgacaaagccatgaagttaggtgctaaccatccaatgggtcctttggaattaggtgactttataggtttggacatctgcttagctattatggatgttttgtattccgaaactggtgacagtaaatacagacctcacacattgttgaagaaatatgttagagcaggttggttaggtagaaagagtggtaaaggtttctacgattactctaaataatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaa

SEQ ID NO: 120 - DNA, 3655 bp, synthetic construct
  misc_feature 1..3655: note = Codon Optimized
  source 1..3655: mol_type = other DNA
tcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagatagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaa

SEQ ID NO: 121 - DNA, 4054 bp, synthetic construct
  misc_feature 1..4054: note = Codon Optimized
  source 1..4054: mol_type = other DNA
tcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgaaaaattgtgtcatagtttctgccgttagaactgctatcggttcttttaacggttcattggcatctacttcagctatcgatttgggtgctacagtaatcaaagctgcaattgaaagagcaaagatagattccttgcatatagacgaagttatcatgggtaatgtattgcaagctggtttaggtcaaaacccagctagacaagcattgttaaaatcaggtttagcagaaaccgtatgtggtttcactgtcaataaggtttgcggttccggtttgaagagtgtcgctttagccgctcaagccattcaagctggtcaagcacaatccatagttgccggtggtatggaaaacatgagtttggcaccttatttgttagatgccaaggctagatctggttatagattaggtgacggtcaagtatacgacgtcattttgagagatggtttaatgtgcgctactcatggttatcacatgggtataactgctgaaaacgttgcaaaggaatacggtatcacaagagaaatgcaagatgaattggctttacactctcaaagaaaggcagccgctgcaatcgaatcaggtgcctttaccgctgaaattgtaccagtcaacgttgtaactagaaagaaaactttcgttttctctcaagacgaatttccaaaagctgattcaactacagaagcattgggtgccttaagacctgccttcgacaaggctggtactgtaactgctggtaatgcatccggtataaacgatggtgccgctgcattggtcatcatggaagaaagtgccgctttagcagccggtttgacacctttagctagaattaaatcctacgcaagtggtggtgtcccacctgctttgatgggtatgggtccagttcctgcaacacaaaagaccttgcaattagcaggtttgcaattagccgatattgacttaatagaagccaatgaagcatttgctgcacaattcttggctgttggtaaaactttaggtttcgactctgaaaaggttaatgtaaacggtggtgcaatcgccttgggtcatccaattggtgcatcaggtgccagaattttagttacattgttacacgctatgcaagcaagagataaaacattgggtttagctaccttgtgtattggtggtggtcaaggtattgcaatggttatagaaagattaaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaa

SEQ ID NO: 122 - DNA, 4054 bp, synthetic construct
  misc_feature 1..4054: note = Codon Optimized
  source 1..4054: mol_type = other DNA
tcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgactagagaagttgtagtcgtttctggtgttagaacagctattggtacttttggtggttcattaaaagatgttgctccagcagaattgggtgcattagtagtcagagaagccttggctagagcacaagtatccggtgacgacgtcggtcatgttgtattcggtaatgttatccaaacagaaccaagagatatgtatttgggtagagtcgctgcagttaatggtggtgtaactattaacgcccctgctttaacagtcaacagattgtgtggttccggtttacaagctatagttagtgccgctcaaacaatcttgttaggtgacaccgacgttgctattggtggtggtgctgaatctatgtcaagagccccatacttagctcctgcagccagatggggtgccagaatgggtgacgctggtttggttgacatgatgttgggtgctttacatgatcctttccatagaatacacatgggtgttactgcagaaaacgtagccaaggaatacgatatttcaagagcacaacaagacgaagctgcattggaatctcacagaagagcatcagccgctatcaaagccggttactttaaggatcaaattgttccagtcgtttcaaaaggtagaaagggtgacgtcaccttcgatactgacgaacatgttagacacgacgctactattgatgacatgacaaaattgagacctgttttcgtaaaggaaaatggtactgttacagccggtaatgcttccggtttgaacgatgcagccgctgcagtagtcatgatggaaagagcagaagccgaaagaagaggtttgaaaccattagctagattggtttcttatggtcatgctggtgttgatccaaaagcaatgggtattggtccagttcctgctaccaagatagcattggaaagagccggtttacaagtctctgatttggacgttatagaagcaaatgaagcctttgccgctcaagcatgtgcagttacaaaagcattgggtttagatccagcaaaagtaaatcctaacggttccggtatcagtttaggtcatccaattggtgctaccggtgcattgattactgttaaggctttacacgaattgaacagagttcaaggtagatacgcattagtaacaatgtgcataggtggtggtcaaggtattgcagccatattcgaaagaatctaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaa

SEQ ID NO: 123 - DNA, 4063 bp, synthetic construct
  misc_feature 1..4063: note = Codon Optimized
  source 1..4063: mol_type = other DNA
tcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgatcgtcaagccaatggttagaaacaacatttgtttgaacgcccatcctcaaggttgcaaaaagggtgttgaagatcaaatcgaatacactaaaaagagaatcacagcagaagtaaaagctggtgcaaaggcccctaaaaatgttttggtattgggttgttccaacggttatggtttggctagtagaataaccgctgcatttggttacggtgccgctactatcggtgtttccttcgaaaaagcaggtagtgaaaccaagtatggtactccaggttggtacaataacttggcctttgatgaagcagccaagagagaaggtttgtactctgtcactatagatggtgacgctttctcagatgaaattaaagcacaagtaatagaagaagccaaaaagaaaggtatcaagttcgatttgatcgtttactctttggcatcaccagtaagaacagatcctgacaccggtataatgcacaagtcagtattgaagccattcggtaaaactttcacaggtaaaaccgtcgatcctttcactggtgaattgaaggaaatatctgcagaaccagccaatgatgaagaagctgcagccactgtcaaagttatgggtggtgaagactgggaaagatggattaaacaattgtccaaggaaggtttgttagaagaaggttgtatcacattggcttactcttacataggtcctgaagctacacaagcattgtatagaaaaggtactatcggtaaagctaaagaacatttggaagctactgcacacagattgaataaggaaaacccatctatcagagcattcgtatcagtcaataagggtttagttacaagagcctccgctgttatcccagtaattcctttgtacttagctagtttgtttaaagttatgaaggaaaagggtaaccatgaaggttgcatagaacaaatcacaagattgtacgcagaaagattgtacagaaaggatggtactattccagttgacgaagaaaacagaattagaatcgatgactgggaattggaagaagacgtacaaaaagccgtctctgctttaatggaaaaggttactggtgaaaacgctgaatcattgacagatttggcaggttatagacacgactttttagcctctaatggtttcgatgtcgaaggtattaactacgaagctgaagttgaaagatttgacagaatataatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaa

SEQ ID NO: 124 - DNA, 5497 bp, synthetic construct
  misc_feature 1..5497: note = Codon Optimized
  source 1..5497: mol_type = other DNA
tcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggtttccaatcacttgtttgacgcaatgagagccgctgcccctggtaacgcccctttcataagaatagataatactagaacttggacatacgatgacgcctttgctttatctggtagaatagcatcagctatggatgctttgggtatcagaccaggtgacagagtcgcagttcaagtagaaaaatccgctgaagcattgatcttgtatttggcttgtttgagaagtggtgcagtttatttgccattgaatactgcctacacattagctgaattggattacttcataggtgacgcagaacctagattggttgtagtcgcctcttcagccagagctggtgtagaaacaattgctaaaccaagaggtgcaatagtcgaaaccttagatgctgctggttctggtagtttgttagatttggccagagacgaacctgctgattttgttgacgcttcaagatcagccgatgacttagccgctattttgtacacctctggtactacaggtagatcaaagggtgctatgttgactcatggtaatttgttgtcaaacgcattaaccttgagagatttctggagagttactgccggtgacagattaatccacgctttgccaatttttcatactcacggtttattcgttgctaccaacgtaactttgttagcaggtgcctccatgttcttgttgagtaagttcgatccagaagaaatattatctttgatgcctcaagctactatgttgatgggtgtcccaacattctacgttagattgttacaatcacctagattagataagcaagctgttgcaaacatcagattgtttatatccggtagtgctccattgttagcagaaacccatactgaatttcaagcaagaacaggtcacgccattttagaaagatacggtatgacagaaaccaatatgaacacttctaacccttatgaaggtaaaagaatagctggtacagttggttttccattgcctgatgtcacagttagagtaaccgacccagccactggtttagctttgccacctgaacaaactggtatgatcgaaattaaaggtccaaacgtttttaagggttactggagaatgcctgaaaagactgctgctgagtttactgctgatggtttctttatctctggtgacttaggtaaaattgatagagacggttatgtccatattgttggtcgtggtaaagatttggttatatccggtggttataacatctaccctaaggaagtagaaggtgaaatagatcaaatcgaaggtgttgtagaatcagctgtaataggtgtcccacatcctgattttggtgaaggtgttacagcagtcgttgtaagaaaaccaggtgctgcattagatgaaaaggcaattgtttctgccttacaagacagattggctagatacaagcaaccaaagagaataatcttcgcagaagatttgcctagaaatactatgggtaaagtacaaaagaacatcttgagacaacaatacgccgacttatacaccagaaccgaaggtagaggttctttgttaacatgtggtgacgttgaagaaaatccaggtcctatggcttcagaaaaggaaataagaagagaaagattcttgaacgtattcccaaagttagttgaagaattgaacgctagtttgttagcttatggtatgcctaaagaagcctgcgattggtatgctcactctttaaactacaatactccaggtggtaaattgaatagaggtttgagtgtagttgatacttatgctatcttgtctaacaaaaccgttgaacaattaggtcaagaagaatacgaaaaggtcgctatcttgggttggtgtattgaattgttgcaagcatactttttggttgccgatgacatgatggataagtctataacaagaagaggtcaaccatgctggtacaaagttccagaagttggtgaaatagccataaatgatgcttttatgttggaagccgctatctataaattgttgaagtcacatttcagaaacgaaaagtactacatcgatattaccgaattattccacgaagttactttccaaacagaattgggtcaattgatggatttgataactgcacctgaagataaagttgacttgtcaaagttttccttgaagaaacattcattcatcgtcacctttgaaactgcttattactccttctatttgccagtcgccttggctatgtacgtagctggtattactgatgaaaaagacttgaagcaagcaagagatgttttgatacctttgggtgaatacttccaaatccaagatgactacttagactgtttcggtactccagaacaaataggtaaaatcggtacagatattcaagacaataagtgcagttgggttattaacaaggctttggaattagcatctgccgaacaaagaaagactttggatgaaaactacggtaaaaaggactcagttgctgaagcaaagtgtaagaaaatttttaatgatttgaagattgaacaattgtaccatgaatacgaagaatccatcgctaaagacttaaaggcaaagattagtcaagttgatgaatcaagaggttttaaagccgacgttttgacagctttcttgaataaggtctacaagagatcaaagtgatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaa

(19) United States
(12) Patent Application Publication
(10) Pub. No.: US 2025/0092427 A1
(43) Pub. Date: Mar. 20, 2025

(54) Fungal Cannabinoids

(71) Applicant: Lygos, Inc., Berkeley, CA (US)

(72) Inventors: Jason L. Poulos, Los Angeles, CA (US); Anthony N. Farina, Pasadena, CA (US)

(21) Appl. No.: 18/759,470

(22) Filed: Jun. 28, 2024

Related U.S. Application Data
(63) Continuation of application No. 17/711,993, filed on Apr. 1, 2022, now Pat. No. 12,043,860.
Application No. 17/711,993 is a continuation of application No. 16/679,637, filed on Nov. 11, 2019, now Pat. No. 11,293,038.
Application No. 16/679,637 is a continuation-in-part of application No. 16/539,436, filed on Aug. 13, 2019, now Pat. No. 10,954,534.
Application No. 16/539,436 is a continuation-in-part of application No. 16/122,702, filed on Sep. 5, 2018, now Pat. No. 10,392,635.
Application No. 16/122,702 is a continuation of application No. 15/815,651, filed on Nov. 16, 2017, now Pat. No. 10,093,949.
Application No. 15/815,651 is a continuation of application No. 14/795,816, filed on Jul. 9, 2015, now Pat. No. 9,822,384.
(60) Provisional application No. 62/024,099, filed on Jul. 14, 2014.

(51) Int. Cl. C12P 7/42; C12N 9/00; C12N 9/10; C12N 9/88; C12N 15/81

(52) U.S. Cl. CPC C12P 7/42; C12N 9/1029; C12N 9/1085; C12N 9/88; C12N 9/93; C12N 15/81; C12Y 203/01206; C12Y 205/01; C12Y 504/99; C12Y 602/01

(57) ABSTRACT

Exemplary embodiments provided herein include genetically engineering microorganisms, such as yeast or bacteria, to produce cannabinoids by inserting genes that produce the appropriate enzymes for the metabolic production of a desired compound.

### CROSS-REFERENCE TO RELATED APPLICATIONS

The present continuation application claims the priority benefit of U.S. Non-Provisional patent application Ser. No. 17/711,993, filed on Apr. 1, 2022, titled “Advanced Production of Cannabinoids in Yeast,” which is a continuation of U.S. Non-Provisional patent application Ser. No. 16/679,637, filed on Nov. 11, 2019, titled “Production of Cannabinoids in Yeast,” which is a continuation-in-part of U.S. Non-Provisional patent application Ser. No. 16/539,436, filed Aug. 13, 2019, titled “Production of Cannabigerolic Acid in Yeast,” now U.S. Pat. No. 10,954,534, issued on Mar. 23, 2021, which is a continuation-in-part of U.S. Non-Provisional patent application Ser. No. 16/122,702, filed Sep. 5, 2018, titled “Production of Tetrahydrocannabinolic Acid in Yeast,” now U.S. Pat. No. 10,392,635, issued on Aug. 27, 2019, which is a continuation of U.S. Non-Provisional patent application Ser. No. 15/815,651, filed Nov. 16, 2017, titled “Production of Cannabidiolic Acid in Yeast,” now U.S. Pat. No. 10,093,949, issued on Oct. 9, 2018, which is a continuation of U.S. Non-Provisional patent application Ser. No. 14/795,816, filed Jul. 9, 2015, titled “Production of Cannabinoids in Yeast,” now U.S. Pat. No. 9,822,384, issued on Nov. 21, 2017, which in turn claims the benefit and priority of U.S. Provisional Patent Application Ser. No. 62/024,099, filed Jul. 14, 2014, titled “Terpenophenolic Production in Microorganisms.” All of the aforementioned disclosures are hereby incorporated by reference herein in their entireties including all references and appendices cited therein.

### REFERENCE TO SEQUENCE LISTINGS

The present application is filed with sequence listing(s) attached hereto and incorporated by reference, including Appendix 1A titled “Additional Examples,” Appendix 1B titled “Sequence IDs,” Appendix 1C titled “Additional Sequence IDs,” and Appendix 1D titled “Sequence IDs.”

### REFERENCE TO AN ELECTRONIC SEQUENCE LISTING

The contents of the electronic sequence listing (18759470.xml; Size: 583,459 bytes; and Date of Creation: Dec. 9, 2024) is herein incorporated by reference in its entirety.

### FIELD OF THE INVENTION

This invention relates to molecular biology, and more specifically to the transformation of yeast cells and the production of cannabinoids.

### SUMMARY OF THE INVENTION

Exemplary embodiments provided herein include genetically engineering microorganisms, such as yeast or bacteria, to produce cannabinoids by inserting genes that produce the appropriate enzymes for the metabolic production of a desired compound.

### BRIEF DESCRIPTION OF THE DRAWINGS

FIG. 1 shows exemplary experimental data achieved in connection with Example 1 of Appendix 1A titled “Additional Examples” for cannabidiol (“CBD”)/cannabidiolic acid (“CBDA”) production in S. cerevisiae.

FIG. 2 shows exemplary experimental data achieved in connection with Example 2 of Appendix 1A titled “Additional Examples” for tetrahydrocannabinol (“THC”)/tetrahydrocannabinolic acid (“THCA”) production in S. cerevisiae.

FIG. 3 shows exemplary experimental data achieved in connection with Example 3 of Appendix 1A titled “Additional Examples” for cannabigerol (“CBG”)/cannabigerolic acid (“CBGA”) production in S. cerevisiae.

FIG. 4 shows exemplary experimental data achieved in connection with Example 4 of Appendix 1A titled “Additional Examples” for CBGA, CBDA, CBD, CBG and THC production in K. marxianus.

FIG. 5 show exemplary experimental data achieved in connection with Example 5 of Appendix 1A titled “Additional Examples” for galactose induced CBD production in S. cerevisiae.

FIG. 6 shows exemplary experimental data achieved in connection with Example 6 of Appendix 1A titled “Additional Examples” for secretion of CBD into media by S. cerevisiae.

FIG. 7 shows an exemplary metabolic pathway for the production of cannabinoids by Cannabis sativa.

FIG. 8 shows an exemplary biosynthetic route for the production of CBDA from glucose.

### DETAILED DESCRIPTION OF EXEMPLARY EMBODIMENTS

The present application relates to the field of cannabinoid production in yeasts. Cannabinoids are a general class of chemicals that act on cannabinoid receptors and other target molecules to modulate a wide range of physiological behavior such as neurotransmitter release. Cannabinoids are produced naturally in humans (called endocannabinoids) and by several plant species (called phytocannabinoids) including Cannabis sativa. Cannabinoids have been shown to have several beneficial medical/therapeutic effects and therefore they are an active area of investigation by the pharmaceutical industry for use as pharmaceutical products for various diseases.

Currently the production of cannabinoids for pharmaceutical or other use is done by chemical synthesis or through the extraction of cannabinoids from plants that are producing these cannabinoids, for example Cannabis sativa. There are several drawbacks to the current methods of cannabinoid production. The chemical synthesis of various cannabinoids is a costly process when compared to the extraction of cannabinoids from naturally occurring plants. The chemical synthesis of cannabinoids also involves the use of chemicals that are not environmentally friendly, which can be considered as an additional cost to their production. Furthermore, the synthetic chemical production of various cannabinoids has been classified as less pharmacologically active as those extracted from plants such as Cannabis sativa. Although there are drawbacks to chemically synthesized cannabinoids, the benefit of this production method is that the end product is a highly pure single cannabinoid. This level of purity is preferred for pharmaceutical use. The level of purity required by the pharmaceutical industry is reflected by the fact that no plant extract based cannabinoid production has received FDA approval yet and only synthetic compounds have been approved.

In contrast to the synthetic chemical production of cannabinoids, the other method that is currently used to produce cannabinoids is production of cannabinoids in plants that naturally produce these chemicals; the most used plant for this is Cannabis sativa. In this method, the plant Cannabis sativa is cultivated and during the flowering cycle various cannabinoids are produced naturally by the plant. The plant can be harvested and the cannabinoids can be ingested for pharmaceutical purposes in various methods directly from the plant itself or the cannabinoids can be extracted from the plant. There are multiple methods to extract the cannabinoids from the plant Cannabis sativa. All of these methods typically involve placing the plant, Cannabis sativa that contains the cannabinoids, into a chemical solution that selectively solubilizes the cannabinoids into this solution. There are various chemical solutions used to do this such as hexane, cold water extraction methods, CO2 extraction methods, and others. This chemical solution, now containing all the different cannabinoids, can then be removed, leaving behind the excess plant material. The cannabinoid containing solution can then be further processed for use.

There are several drawbacks of the natural production and extraction of cannabinoids in plants such as Cannabis sativa. Since there are numerous cannabinoids produced by Cannabis sativa it is often difficult to reproduce identical cannabinoid profiles in plants using an extraction process. Furthermore, variations in plant growth will lead to different levels of cannabinoids in the plant itself making reproducible extraction difficult. Different cannabinoid profiles will have different pharmaceutical effects which are not desired for a pharmaceutical product. Furthermore, the extraction of cannabinoids from Cannabis sativa extracts produces a mixture of cannabinoids and not a highly pure single pharmaceutical compound. Since many cannabinoids are similar in structure it is difficult to purify these mixtures to a high level resulting in cannabinoid contamination of the end product.

Disclosed herein are strategies for creating cannabinoids in microorganisms such as yeast and methods to produce various cannabinoids in yeast from a simple sugar source. The general methods involve genetically engineering yeast to produce various cannabinoids, where the main carbon source available to the yeast is a sugar (glucose, galactose, fructose, sucrose, honey, molasses, raw sugar, etc.). Genetic engineering of the microorganism involves inserting various genes that produce the appropriate enzymes and/or altering the natural metabolic pathway in the microorganism to achieve the production of a desired compound. Through genetic engineering of microorganisms these metabolic pathways can be introduced into these microorganisms and the same metabolic products that are produced in the plant Cannabis sativa can be produced by the microorganisms. The benefit of this method is that once the microorganism is produced, the production of the cannabinoid is low cost and reliable, only a specific cannabinoid is produced or a subset is produced, depending on the organism. The purification of the cannabinoid is straight forward since there is only a single cannabinoid or a selected few cannabinoids present in the microorganism. The process is a sustainable process which is more environmentally friendly than synthetic production.

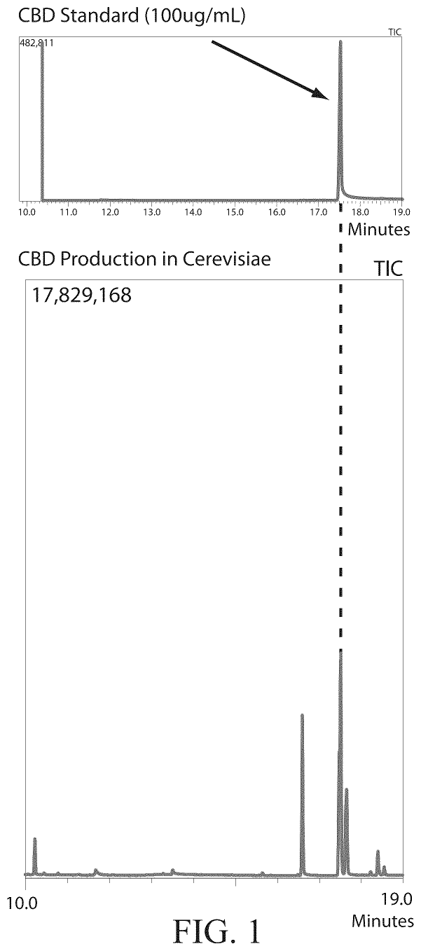

FIG. 1 shows exemplary experimental data achieved in connection with Example 1 of Appendix 1A titled “Additional Examples” for cannabidiol (“CBD”)/cannabidiolic acid (“CBDA”) production in S. cerevisiae.

FIG. 1 shows gas chromatography-mass spectrometry of cannabidiol (CBD) produced in S. cerevisiae. After processing the yeast cells, as described in Example 1 of Appendix 1A, the whole cell ethyl acetate extract is analyzed for the presences of CBD. The samples were prepared in a way similar to that shown in Appendix A1 except that no MSTFA derivatization was used in this sample (therefore CBDA turns into CBD upon heating), the oven protocol was also slightly different than that shown in Appendix 1A. Initially, a standard CBD solution is run (100 ug/mL; TOP). After running the standard the inventors determined the run time of 17.5 minutes. After running the standard the inventors repeated the GC-MS experiment with their whole cell extract (BOTTOM). At a run time of 17.5 minutes, the inventors saw the same peak as in the standard. Mass spectrometry analysis of the peaks showed identical mass for the two samples (standard and extract) indicating the presence of CBD in their whole cell extract.

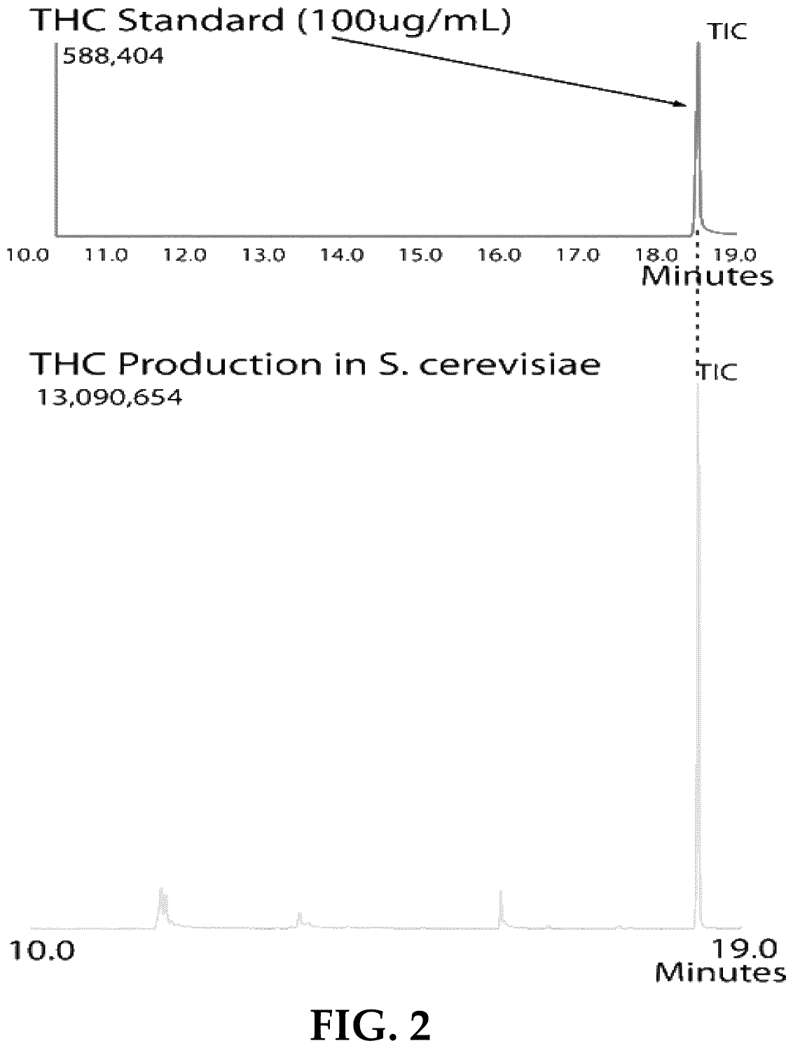

FIG. 2 shows exemplary experimental data achieved in connection with Example 2 of Appendix 1A titled “Additional Examples” for tetrahydrocannabinol (“THC”)/tetrahydrocannabinolic acid (“THCA”) production in S. cerevisiae.

FIG. 2 shows gas chromatography-mass spectrometry of tetrahydrocannabinol (THC) produced in S. cerevisiae. After processing the yeast cells, as described in Example 2 of Appendix 1A, the whole cell ethyl acetate extract was analyzed for the presences of THC. The samples were prepared in a way similar to that shown in Appendix 1A except that no MSTFA derivatization was used in this sample (therefore THCA turns into THC upon heating), the oven protocol was also slightly different than that shown in Appendix 1A. Initially, a standard CBD solution was run (100 ug/mL; TOP). After running the standard the inventors determined the run time of 18.5 minutes. After running the standard the inventors repeated the GC-MS experiment with their whole cell extract (BOTTOM). At a run time of 18.5 minutes, the inventors saw the same peak as in the standard. Mass spectrometry analysis of the peaks showed identical mass for the two samples (standard and extract) indicating the presence of THC in their whole cell extract.

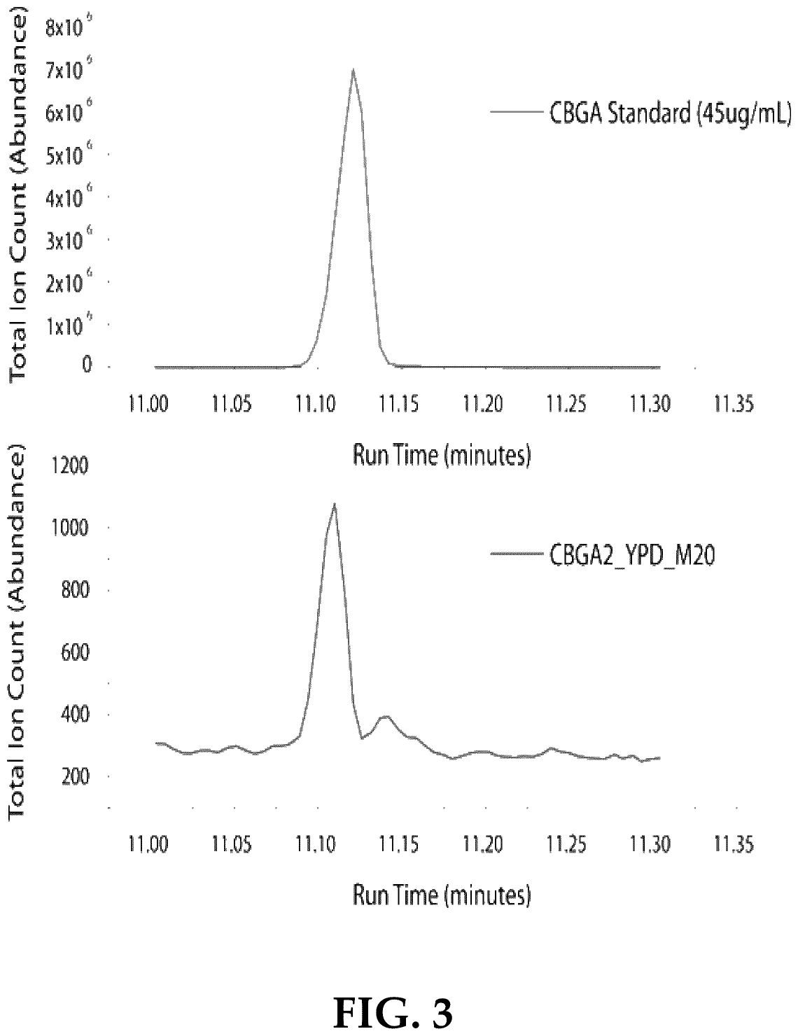

FIG. 3 shows exemplary experimental data achieved in connection with Example 3 of Appendix 1A titled “Additional Examples” for cannabigerol (“CBG”)/cannabigerolic acid (“CBGA”) production in S. cerevisiae.

FIG. 3 shows gas chromatography-mass spectrometry of cannabigerolic acid (CBGA) produced in S. cerevisiae. After processing the yeast cells, as described in Example 3 of Appendix 1A, the whole cell ethyl acetate extract was analyzed for the presences of CBGA. The samples were prepared in a way as described in Appendix 1A, but the oven protocol was also slightly different than that shown in Appendix 1A. Initially, a standard CBGA solution was run (45 ug/mL; TOP). After running the standard, the inventors determined the run time of 11.1 minutes. After running the standard, the inventors repeated the GC-MS experiment with their whole cell extract (BOTTOM). At a run time of 11.1 minutes, the inventors saw the same peak as in the standard. Mass spectrometry analysis of the peaks showed identical mass for the two samples (standard and extract) indicating the presence of CBGA in their whole cell extract.

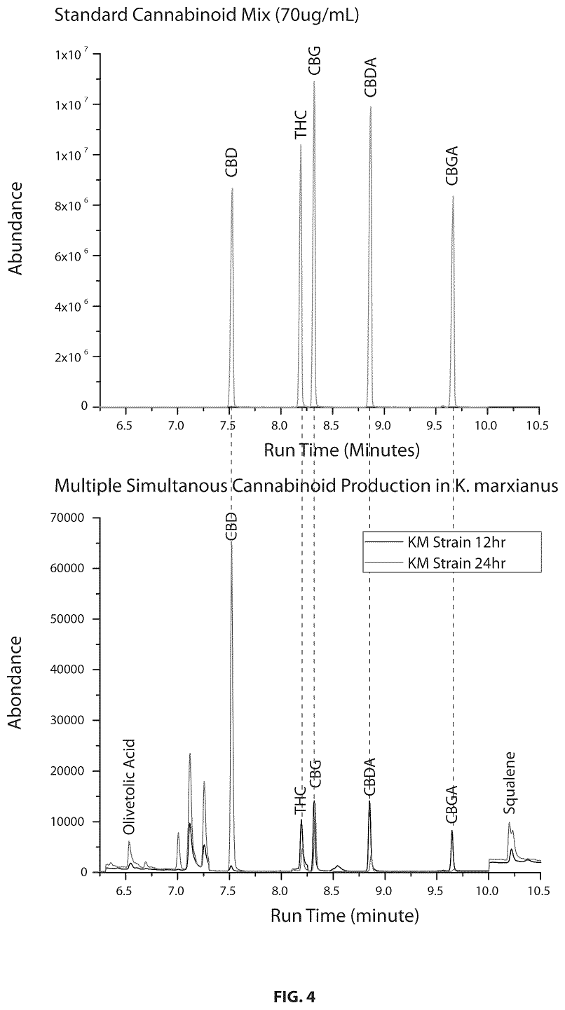

FIG. 4 shows exemplary experimental data achieved in connection with Example 4 of Appendix 1A titled “Additional Examples” for CBGA, CBDA, CBD, CBG and THC production in K. marxianus.

FIG. 4 shows gas chromatography-mass spectrometry of cannabinoid production (CBGA, CBDA, CBD, CBG, THC) produced in K. marxianus. After processing the yeast cells, as described in Example 4 of Appendix 1A, the whole cell ethyl acetate extract was analyzed for the presence of cannabinoids. The samples were prepared in a way as described in Appendix 1A, but the oven protocol was also slightly different than that shown in Appendix 1A. Initially, a standard solution containing CBD, CBG, THC, CBDA, and CBGA was run (70 ug/mL each; TOP). After running the standard, the inventors determined the run time for each compounds. After running the standard, the inventors repeated the GC-MS experiment with their whole cell extract (BOTTOM). At each run time the inventors saw the same peaks as in the standard. Mass spectrometry analysis of the peaks showed identical mass for the two samples (standard and extract) indicating the presence of cannabinoids in their whole cell extract.

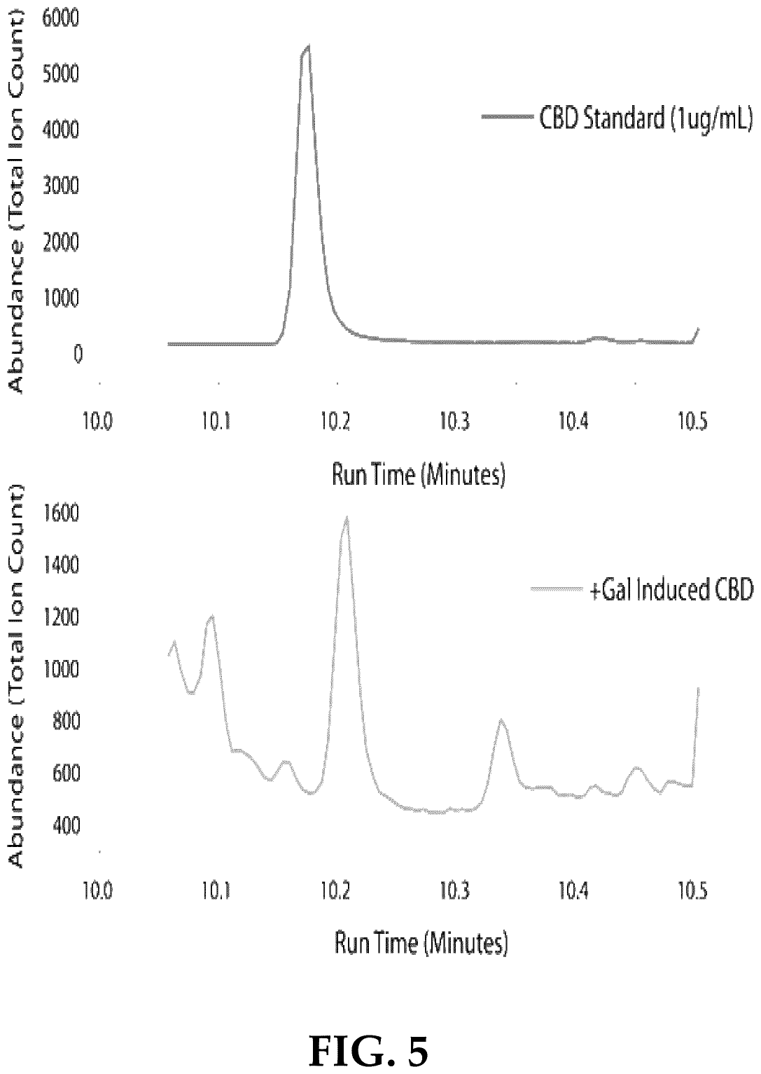

FIG. 5 show exemplary experimental data achieved in connection with Example 5 of Appendix 1A titled “Additional Examples” for galactose induced CBD production in S. cerevisiae.

FIG. 5 shows gas chromatography-mass spectrometry of induced cannabidiol (CBD) production in S. cerevisiae. After processing yeast cells, as described in Example 5 of Appendix 1A, the whole cell ethyl acetate extract was analyzed for the presences of CBD. The samples were prepared in a way as described in Appendix 1A. Initially, a standard solution containing CBD was run (1 ug/mL; TOP). After running the standard the inventors determined the run time for CBD as 10.2 minutes. After running the standard, the inventors repeated the GC-MS experiment with their whole cell extract (BOTTOM). At 10.2 minutes, the inventors saw the same peak as in the standard. Mass spectrometry analysis of the peaks showed identical mass for the two samples (standard and extract) indicating the presence of CBD in their whole cell extract.

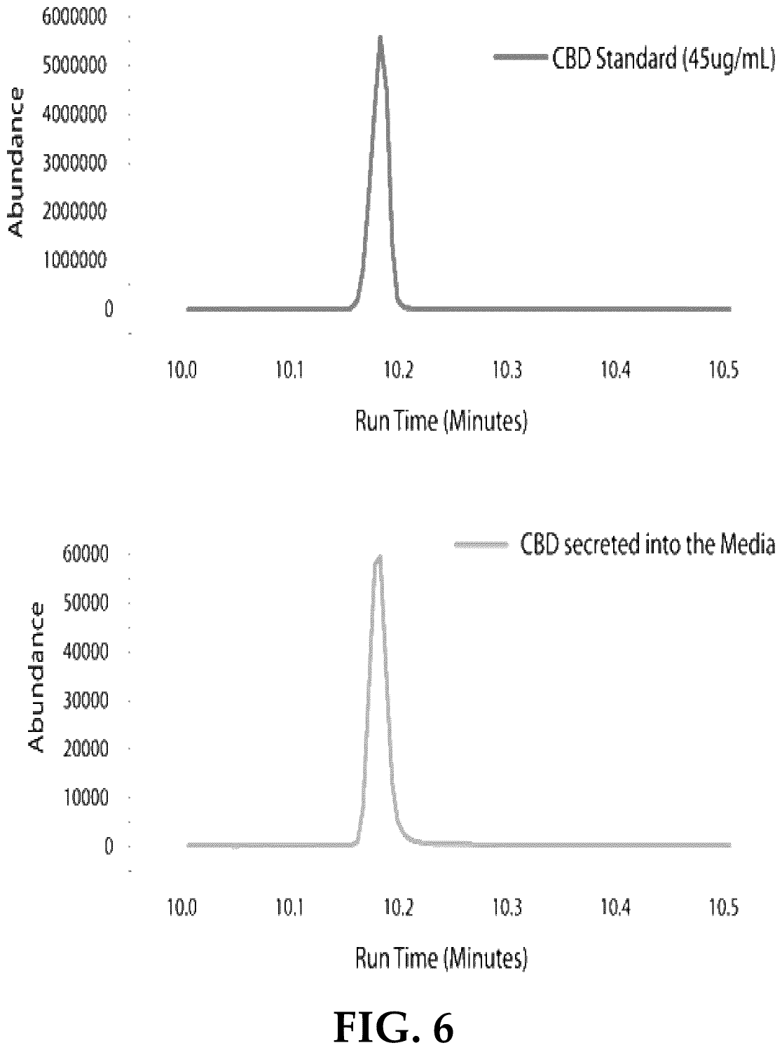

FIG. 6 shows exemplary experimental data achieved in connection with Example 6 of Appendix 1A titled “Additional Examples” for secretion of CBD into media by S. cerevisiae.

FIG. 6 shows gas chromatography-mass spectrometry of induced cannabidiol production (CBD) produced in S. cerevisiae and secreted into the media. After processing the growth media, as described in Example 6 of Appendix 1A, the media ethyl acetate extract was analyzed for the presence of CBD. The samples were prepared in a way as described in Appendix 1A. Initially, a standard solution containing CBD was run (45 ug/mL; TOP). After running the standard the inventors determined the run time for CBD as 10.2 minutes. After running the standard, the inventors repeated the GC-MS experiment with their whole cell extract (BOTTOM). At 10.2 minutes, the inventors saw the same peak as in the standard. Mass spectrometry analysis of the peaks showed identical mass for the two samples (standard and extract) indicating the presence of CBD in their whole cell extract.

Biosynthetic Production of Cannabidiolic Acid (CBDA) in S. cerevisiae.

Through genetic engineering the inventors have reconstituted the cannabidiolic acid (CBDA) metabolic pathway found in Cannabis sativa into S. cerevisiae (a species of yeast).

Producing CBGA is an initial step in producing many cannabinoids from Cannabis sativa in S. cerevisiae. Once CBGA is produced a single additional enzymatic step is required to turn CBGA into many other cannabinoids (CBDA, THCA, CBCA, etc.). The acidic forms of the cannabinoids can be used as a pharmaceutical product or the acidic cannabinoids can be turned into their neutral form for use, for example Cannabidiol (CBD) is produced from CBDA through decarboxylation. The resulting cannabinoid products will be used in the pharmaceutical/nutraceutical industry to treat a wide range of health issues.

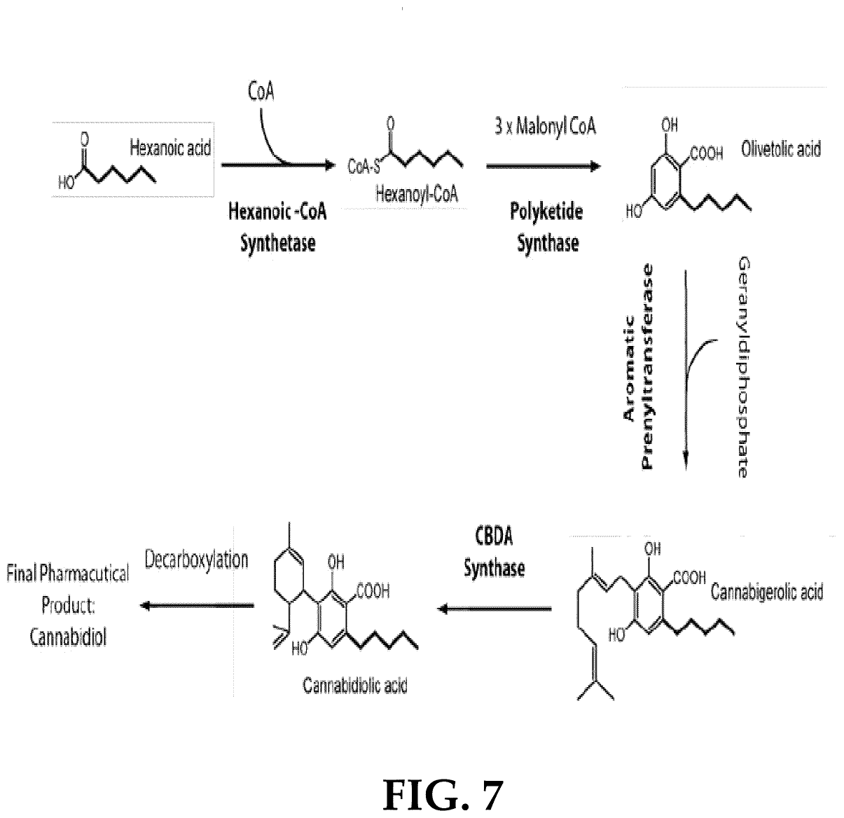

FIG. 7 shows an exemplary metabolic pathway for the production of cannabinoids by Cannabis sativa.

The biosynthetic route for the production of cannabidiolic acid in Cannabis sativa is shown in FIG. 7. The pathway begins with the conversion of Hexanoic acid (a simple fatty acid) to Hexanoyl-CoA by Hexanoyl-CoA Synthetase. Hexanoyl-CoA is converted to Oleviolic acid (OA), a polyketide, by a Polyketide synthase. OA is then prenylated with the monoterpene geranyl diphosphate to cannabigerolic acid by an Aromatic prenyltransferase. Finally, cannabidiolic acid (CBDA) is produced by cyclizing cannabigerolic acid via CBDA synthase (CBDAs). In summary, it takes four enzymatic steps to produce CBDA from Hexanoic acid. The inventors have engineered this metabolic pathway into S. cerevisiae (a species of yeast) for the production of CBDA.

There are a few key differences between plant polyketide and terpene biosynthesis when compared to yeast. Yeast does not contain many of the enzymes and fatty acids required for the production of CBDA. Moreover, yeast do not express high levels of geranyl diphosphate (GPP), a chemical required for the production of cannabigerolic acid, the precursor to CBDA. Yet, through genetic engineering many of the required enzymes can be added and the production of GPP can be increased. In order to add the required enzymes for CBDA production in yeast the inventors created plasmids that contain the essential genes in the CBDA biosynthetic pathway. The inventors have transformed these genes into S. cerevisiae.

Synthesis of Fusion Genes Required for CBDA Production in S. cerevisiae.

The genome of Cannabis sativa has been investigated and the acyl-activating enzymes CsAAE1 was determined to convert hexanoic acid to hexanoyl-CoA (Step 1 in FIG. 7). The inventors have overexpressed CsAAE1 in yeast while simultaneously supplementing the growth media with Hexanoic acid. By supplementing the media with hexanoic acid, the inventors ensured that the yeast have the required starting materials for the production of hexanoyl-CoA. In addition to CsEE1, the cannabis plant has several other acyl-activating enzymes with sequences that are similar to CsEE1. These enzymes can also be used for the conversation of hexanoic acid to hexanoyl-CoA. These are listed in Appendix 1C.

The next enzymatic step that was engineered into the yeast strain was for the production of Olivetolic acid (OA) from hexanoyl-CoA. This step requires the substrates hexanoyl-CoA and 3 malonyl-CoA molecules, with the malonyl-CoA molecule produced by yeast naturally. Olivetolic acid production requires two enzymes for the condensation and subsequent cyclization of malonyl-CoA with hexanoyl-CoA. This process requires the tetraketide synthase, olivetolic synthase (OS), and the polyketide cyclase, olivetolic acid cyclase (OAC). In some embodiments, stoichiometric amounts of both of these enzymes are preferred; as it has been experimentally determined that OAC binds a chemical intermediate made by OS. In various embodiments, in order to ensure the proper amounts of OS and OAC the inventors have created a single gene that is a fusion of OS, a self cleaving T2A peptide, and the OAC gene (OS-T2A-OAC) and in certain cases an HA tag was inserted at the C-terminus of OAC to verify protein expression. This entire fusion protein was produced in yeast and the self cleaving peptide is spliced in vivo to produce OS and OAC. In addition to the tetraketide synthase, olivetolic synthase (OS), and the polyketide cyclase, olivetolic acid cyclase (OAC), the cannabis plant has several other enzymes with sequences that are similar to the tetraketide synthase, olivetolic synthase (OS), and the polyketide cyclase, olivetolic acid cyclase (OAC). These enzymes can also be used for the conversation of hexanoyl-CoA to olivetolic acid. These are listed in Appendix 1C.

The next enzymatic step requires the production of geranyl pyrophosphate (GPP). In yeast the prenyltransferase Erg20 condenses isopentenyl diphosphate (IPP) and dimethylallyl diphosphate (DMAP) to geranyl pyrophosphate (GPP) and feranyl pyrophosphate (FPP) naturally. While only these two products are produced in yeast, a greater quantity of FPP when compared to GPP is produced. More GPP is required for the production of CBDA. In order to increase the production of GPP compared to FPP the inventors inserted a mutant prenyltransferase, Erg20(K179E) in the yeast strain. This mutant has been shown to shift the ratio of GPP:FPP to 70:30. This Erg20(K179E) mutant was placed on a fusion gene with CsAAE1, the enzyme for hexanoyl-CoA, and a self-cleaving peptide, T2A (CsAAE1-T2A-Erg20(K179E). We also added a FLAG tag to the C-terminus of the Erg20p (K197E) enzyme (CsAAE1-T2A-Erg20(K179E)-FLAG) to verify expression of this fusion protein in yeast in certain yeast strains. After production in yeast the self-cleaving peptide was cut producing CsAAE1 and Erg20(K179E).

Once the inventors verified that they had enough GPP to prenylate Olivetolic acid to cannabigerolic acid the inventors inserted the aromatic prenyltransferase (CsPt1) gene into the yeast. In this final enzymatic step the inventors placed the cannabidiolic acid synthase (CBDAs) gene into yeast for the conversion of cannabigerolic acid to CBDA. Similar to the inventors' previous approach, they introduced a single gene containing CsPt1, a self-cleaving peptide T2A, CBDs, and in certain cases a MYC tag was inserted at the C-terminus of CBDs in order to verify production of each enzyme (CsPt1-T2A-CBDs-MYC). In addition to the aromatic prenyltransferase (CsPt1) the cannabis plant has several other enzymes with sequences that are similar to the aromatic prenyltransferase (CsPt1). These enzymes can also be used for the conversation of olivetolic acid to CBGA. These are listed in Appendix 1C.

### Creation of a Stable Yeast Strain Producing the Metabolic Pathway for CBDA.

Three stable transformations of S. cerevisiae where created utilizing selection for leucine, uracil and tryptophan. The inventors first transformed an auxotrophic yeast strain (his3D1/leu2/trp1-289/ura3-52) with the CsAAE1-T2A-Erg20(K197E)-FLAG gene in an integrating vector; other sequences listed in Appendix 1C number 85-91 can replace CsAAE1. 5 μg of CsAAE1-T2A-Erg20(K197E)-FLAG in a vector containing a gene for tryptophan depletion resistance was linearized with the restriction enzyme EcoRV, transformed into chemically competent InVSc1, and grown on Yeast Nitrogen Base without amino acids and 0.5% ammonium sulfate (YNBA) agar plates supplemented with histidine, leucine, tryptophan, 1% glucose and 2% lactic acid are grown at 30° C. until colonies are formed. Any yeast colonies that did not incorporate the plasmid, that contains the CsAAE1-T2A-Erg20(K197E)-FLAG gene died since the starting yeast strain is a tryptophan auxotroph. All colonies, with successful plasmid incorporation, where picked and grown in YNBA supplemented with histidine, leucine and uracil, 1% glucose and 2% lactic acid. All colonies were screened for protein production by taking whole cell extracts of each induced clone and the total protein was subjected to SDS-PAGE followed by western blotting against the c-terminal tag of Erg20(K197E). Positive clones where stored at −80° C. in glycerol stocks. The highest expressing clone was taken for the second transformation and was designated as VscGPHA.

Using the VscGPHA strains the inventors added 5 μg of OS-T2A-OAC-HA in the a vector containing a gene for leucine depletion resistance; other sequences listed in Appendix 1C number 60-84 can replace OS and OAC. This plasmid was linearized with the restriction enzyme AseI and transformed into chemically competent VscGPHA and grown on YNBA agar plates supplemented with histidine and uracil, 1% glucose and 2% lactic acid and grown at 30° C. until colonies were formed. Any yeast colonies that did not incorporate the plasmid that contains the OS-T2A-OAC-HA gene died since the VscGPHA is a leucine auxotroph. All colonies, with successful plasmid incorporation, were picked and grown in YNBA supplemented with histidine, and leucine. All colonies were screened for protein production by taking whole cell extracts of each induced clone and subjected the total protein to SDS-PAGE followed by western blotting against the c-terminal HA tag of OAC. Positive clones were stored at −80° C. in glycerol stocks. The highest expressing clone was taken for the second stable transformation and was designated VscGPHOA.

The final stable transformation was done in a similar way as the previous transformation. The CsPT-T2A-CBDAs-MYC gene was placed in the vector containing a gene for uracil depletion resistance 5 μg of this plasmid was linearized with EcorV and transformed into chemically competent VscGPHOA; other sequences listed in Appendix 1C number 30-59 can replace CsPT. Transformed VscGPHOA was grown in YNBA supplemented with histidine, 1% glucose and 2% lactic acid. Any yeast colonies that did not incorporate the plasmid that contains the CsPT-T2A-CBDAs-MYC gene died since they lacked leucine. All colonies were picked and grown in YNBA supplemented with histidine, 1% glucose and 2% lactic acid. All colonies were screened for protein production by taking whole cell extracts of each induced clone and subjecting the total protein to SDS-PAGE followed by western blotting against the c-terminal Myc tag of CBDAs. Positive clones are stored at −80° C. in glycerol stocks. The highest expressing CBDAs was taken for the final strain and designated VscCBDA.

### Production of CBDA in Yeast.

To initiate the reconstituted metabolic pathway of CBDA a colony of VscCBDA was freshly streaked on a plate of a frozen glycerol stock of VscCBDA. A small culture of VscCBDA was grown in YNBA supplemented with 0.05% histidine, 1% glucose, 2% lactic acid, and 0.03% hexanoic acid overnight at 30° C. The overnight culture was transferred to 1 L of YNBA supplemented with 0.05% histidine, 1% glucose, 2% lactic acid, and 0.03% hexanoic acid and was grown at 30° C. until mid-log phase. Cells were pelleted by centrifugation then washed with 200 ml of phosphate buffered saline (PBS) and repelleted. Pelleted cells were resuspended with 1 L of YNBA supplemented with 0.05% histidine, 2% galactose, and 0.03% hexanoic acid and grown at 30° C. overnight.

Overnight 1 L cultures were pelleted by centrifugation, resuspended, washed one time in PBS and pelleted. Cell pellets were resuspended in 40% (wt/vol) KOH and 50% (vol/vol) ethanol solution and boiled for 10 minutes. Metabolite extraction was done by extracting from the boiled extracts 3 times with hexane, then 3 times with ethyl acetate. The spent supernatant broth was extracted in a similar fashion as described above. Organic phases of extracts of each sample were pooled then dried by a rotary evaporator and stored for liquid chromatography mass spectrometry (LC-MS) and gas chromatography mass spectrometry (GC-MS) analysis to confirm and quantitate how much CBDA is produced from strain VscCBDA.

Biosynthetic Production of Cannabidiolic Acid (CBDA) in K. marxianus.

Through genetic engineering the inventors have reconstituted the cannabidiolic acid (CBDA) metabolic pathway found in Cannabis sativa into K. marxianus (a species of yeast). Producing CBGA is an initial step in producing many cannabinoids from Cannabis sativa in K. marxianus. Once CBGA is produced a single additional enzymatic step is required to turn CBGA into many other cannabinoids (CBDA, THCA, CBCA, etc.). The acidic forms of the cannabinoids can be used as a pharmaceutical product or the acidic cannabinoids can be turned into their neutral form for use, for example Cannabidiol (CBD) is produced from CBDA through decarboxylation. The resulting cannabinoid products will be used in the pharmaceutical/nutraceutical industry to treat a wide range of health issues.

### Synthesis of Fusion Genes Required for CBDA Production in K. Marxianus.

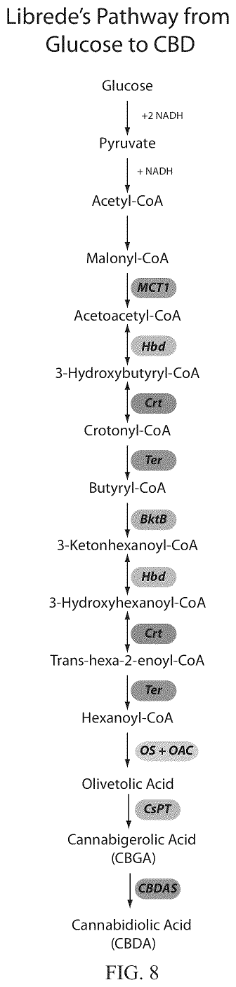

FIG. 8 shows an exemplary biosynthetic route for the production of CBDA from glucose. The biosynthetic route for the production of cannabidiolic acid in Cannabis sativa, from glucose to CBDA is shown in FIG. 8. The pathway begins with the conversion of glucose to malonyl-CoA through a series of steps that are common to many strains of yeast. The conversion of malonyl-CoA to Acetoacetyl-CoA is conducted by the enzyme MCT1, an acyl-carrier-protein. Acetoacetyl-CoA is converted to 3-Hydroxybutyryl-CoA by the enzyme 3-hydroxybutyryl-CoA dehydrogenase (Hbd) from Clostridium acetobutylicum. Next, 3-Hydroxybutyryl-CoA is converted into Crotonyl-CoA by the enzyme crotonase (Crt) from Clostridium acetobutylicum and the conversion of Crotonyl-CoA to Butyryl-CoA is controlled by the enzyme trans-enoyl-CoA reductase (Ter) from Treponema denticola. The Butyryl-CoA is converted to 3-Ketonhexanoyl-CoA by the enzyme β-ketothiolase (Bktb) from Ralstonia Eutropha. 3-Ketonhexanoyl-CoA is converted to 3-Hydroxyhexanoyl-CoA by the enzyme Hbd. Hydroxyhexanoyl-CoA is converted to Trans-hexa-2-enoyl-CoA by the enzyme Crt. Trans-hexa-2-enoyl-CoA is converted to Hexanoyl-CoA by the enzyme Ter. Hexanoyl-CoA, with 3 malonyl-CoAs, is converted to Oleviolic acid (OA) by a Polyketide synthase and cyclase, OA and OAC respectively. OA is then prenylated with the monoterpene geranyl diphosphate to cannabigerolic acid by an Aromatic prenyltransferase, CsPT. Finally, cannabidiolic acid (CBDA) is produced by cyclizing cannabigerolic acid via CBDA synthase (CBDAS). We have engineered this metabolic pathway into K. marxianus (a species of yeast) for the production of CBDA (FIG. 8).

There are a few key differences between plant polyketide and terpene biosynthesis when compared to yeast. Yeast does not contain many of the enzymes and fatty acids required for the production of CBDA. Moreover, yeast do not express high levels of geranyl diphosphate (GPP), a chemical required for the production of cannabigerolic acid, the precursor to CBDA. Yet, the inventors through genetic engineering created many of the required enzymes that can be added so the production of GPP was increased. In order to add the required enzymes for CBDA production in yeast the inventors created plasmids that contain the essential genes in the CBDA biosynthetic pathway. The inventors have transformed these genes into K. marxianus.

Creation of a Stable K. marxianus Strain Producing the Metabolic Pathways for Hexonyl-coA and CBDA.

Two stable transformations of K. marxianus were created utilizing selection for uracil and G418 (Genenticin). The inventors first transformed an auxotrophic K. marxianus strain (ATCC 17555 KM5) with 5 different genes needed to produce high levels of hexanoyl-CoA. After functional conformation of the genes required for hexanoyl-CoA the inventors did a second transformation with the genes responsible for CBDA production. The molecular biology methods required for biosynthetic production of CBDA in K. marxianus are outlined below.

Gene names Crt, Bktb, MCT1, TeR, Hbd, Erg20p(K179E) were codon optimized, synthesized and subcloned into puc57 and p426 ATCC with the restriction enzymes SpeI and SalI.

Genes Crt, Bktb, MCT1, TeR, Hbd, and Erg20p(K179E) were amplified via PCR using the primers GPD_F and URA_R and all 6 amplicons were electroporated into K. marxianus ATCC 17555 KM5 at a concentration of 200 nM and selected onto yeast nitrogen base (YNB) supplemented with amino acid dropout mix (DO supplement—Ura Clonetech 630416) 2% glucose, and 2% Agar plates.

Gene integration and functional gene expression of Crt, Bktb, MCT1, TeR, Hbd, and Erg20p(K179E) was validated by genomic PCR and RT-PCR methods respectively. The final strain produced containing the functional expression of Crt, Bktb, MCT1, TeR, Hbd, and Erg20p(K179E) was labeled kMarxHex1.

Gene names CBDAs, CsPt, OS, and OAC were codon optimized and synthesized by Genscript. The codon optimized gene sequences of CBDAs and CsPt were cloned in frame with a nucleotide sequence containing a self cleaving T2A peptide and designated as CstTCbds. The codon optimized gene sequences of OS and OAC were cloned in frame with a nucleotide sequence containing a self cleaving T2A peptide and designated as OSTOc. CsTCbds and OSTOc were cloned in frame with an S. cerevisiae internal ribosomal entry site (IRES), Ure2, into a galactose inducible vector and the final gene sequence pcen/arsGal-OSTOc-IRES-CsTCbds plasmid can be seen below. The plasmid pcen/arsGal-OSTOc-IRES-CstTCbds was used to synthesize a functional gene fragment that expresses the enzymes CBDAs, CsPt, OS, and OAC by using the primers GalIRES_F, GalIRES_R.

The Gibson Assembly method was used to subclone the PCR fragment from into the plasmid HO-polyKanMx4-HO (ATCC 87804) using the primers KmXIRES_F and KmXIRES_R to create the plasmid pHOOSCstKnMxHO.

The plasmid pHOOSCstKnMxHO was digested with NotI and transformed into kMarxHex1 using standard electroporation methods. The selection of stable integrants was done with yeast nitrogen base (YNB) supplemented with amino acid dropout mix (DO supplement-Ura Clonetech 630416) 2% glucose, 1 mg/ml G418 (Gibco) and 2% Agar plates.

Gene integration and functional gene expression of pHOOSCstKnMxHO validated by genomic PCR and RT-PCR methods respectively. The final strain produced containing the functional expression of Crt, Bktb, MCT1, TeR, Hbd, Erg20p(K179E) CBDAs, CsPt, OS, and OAC was labeled k.MarxCBDA.

Production of CBDA in K. marxianus.

To initiate the reconstituted metabolic pathway of CBDA, a colony from k.Marx CBDA was freshly streaked onto an agar plate from a frozen glycerol stock of k.Marx CBDA. A small culture of VscCBDA was grown in YNBA base (YNB) supplemented with amino acid dropout mix (DO supplement-Ura Clonetech 630416) 2% glucose, 1 mg/ml G418 (Gibco) and 2% Agar plates was grown overnight at 30° C. The overnight culture was transferred to 1 L of YNBA supplemented with base (YNB) supplemented with amino acid dropout mix (DO supplement-Ura Clonetech 630416) 2% glucose, and 1 mg/ml G418 (Gibco) and was grown at 30° C. until mid-log phase. Cells were pelleted by centrifugation then washed with 200 ml of phosphate buffered saline (PBS) and repelleted. Pelleted cells were resuspended with 1L of YNBA supplemented with base (YNB) supplemented with amino acid dropout mix (DO supplement—Ura Clonetech 630416) 2% galactose, and 1 mg/ml G418 (Gibco) and grown at 30° C. overnight.

### Processing CBDA for Analysis of Cannabinoid Production.

Overnight 1 L cultures were pelleted by centrifugation, re-suspended, washed one time in PBS and pelleted. The process for extracting cannabinoids from the yeast generally follows the following basic steps:
1. Remove the yeast cells from the media by centrifugation or filtration.
2. Lysis the cells using either chemical or mechanical methods or a combination of methods. Mechanical methods can include a French Press or glass bead milling or other standard methods. Chemical methods can include enzymatic cell lysis, solvent cell lysis, or detergent based cell lysis.
3. Perform a liquid-liquid extraction of the cannabinoids form the cell lysate using the appropriate chemical solvent. An appropriate solvent is any solvent where the cannabinoids are highly soluble in this solvent and the solvent is not miscible in water. Examples of this are hexane, ethyl acetate, and cyclohexane. Preferred solvents can be straight or branched alkane chains (C5-C8) work well; mixtures of these solvents can also be use.
   Protocol Used for Cannabinoid Extraction from Yeast Cell Lysate
1. After lysising the cells using any mechanical technique, add 1 mL of 4M KCl, pH2.0 to each 1 mL of cell lysate.
2. Add 1-2 mLs of ethyl acetate for each 1 mL of cell lysate.
3. Rigorously mix for 1 min.
4. Centrifuge the mixture for 5 min at 1000×g.
5. Remove the top ethyl acetate layer. Cannabinoids are present in this layer.
   a. The ethyl acetate can be removed under vacuum if desired.
   b. Cannabinoids can be further purified through liquid chromatography methods if desired.
      Protocol Used for Cannabinoid Extraction from Growth Media (for Secreted Cannabinoid Samples)
1. Add 1 mL of ethyl acetate for every 1 mL of growth media.
2. Rigorously mix for 1 min.
3. Centrifuge the mixture for 5 min at 1000×g.
4. Remove the top ethyl acetate layer. Cannabinoids are present in this layer.
   a. The ethyl acetate can be removed under vacuum if desired.
   b. Cannabinoids can be further purified through liquid chromatography methods if desired.

### Protocol Used for GC-MS Analysis of Cannabinoid Extracts for k.Marx CBDA

1. Remove solvent from samples under vacuum.
2. Re-suspend dry samples in either 100 μL of dry hexane or dry ethyl acetate
3. Add 20 μL of N-Methyl-N-(trimethylsilyl) trifluoroacetamide (MSTFA)
4. Briefly mix
5. Heat solution to 60° C. for 10-15 minutes
6. GC-MS Method
   a. Instrument Agilent 6890-5975 GC-MS (Model Number: Agilent 19091S-433)
   b. Column HP-5 MS 5% Phenyl Methyl Siloxane
   c. OVEN:

| | |
|---|---|
| i. Initial temp: 100 ′C. (On) | Maximum temp: 300 ′C. |
| ii. Initial time: 3.00 min | Equilibration time: 0.50 min |

iii. Ramps:

| # | Rate | Final temp | Final time |
|---|---|---|---|
| 1 | −30.00 | 280 | 1.00 |
| 2 | −70.00 | 300 | 5.00 |
| 3 | −0.0(Off) | | |

iv. Post temp: 0° C.
v. Post time: 0.00 min
vi. Run time: 15.29 min

What is claimed is:

1. A method for making Cannabigerovarinic Acid in yeast, the method comprising:
converting glucose to malonyl-CoA with a first nucleotide sequence comprising the nucleotide sequence of SEQ. ID. NO.124 expressing Meningitis associated and temperature regulated Fimbria (MatB);
converting malonyl-CoA to Acetoacetyl-CoA with a second nucleotide sequence comprising the nucleotide sequence of SEQ. ID. NO.118 expressing Monocarboxylate Transporter 1 (MCT1);
converting Acetoacetyl-CoA to 3-Hydroxybutyryl-CoA with a third nucleotide sequence comprising the nucleotide sequence of SEQ. ID. NO. 119 expressing 3-hydroxybutyryl-CoA dehydrogenase (Hbd);
converting 3-Hydroxybutyryl-CoA to Crotonyl-CoA with a fourth nucleotide sequence comprising the nucleotide sequence of SEQ. ID. NO. 120 expressing crotonase (Crt);
converting Crotonyl-CoA to Butyryl-CoA with a fifth nucleotide sequence comprising the nucleotide sequence of SEQ. ID. NO.123 expressing trans-enoyl-CoA reductase (Ter);
converting Butyryl-CoA to divarinic acid with a sixth nucleotide sequence comprising the nucleotide sequence of SEQ. ID. NO. 60 expressing Polyketide synthase and with a seventh nucleotide sequence of SEQ. ID. NO. 92 or SEQ. ID. NO. 93 expressing olivetolic acid cyclase; and
converting divarinic acid to Cannabigerovarinic Acid with an eighth nucleotide sequence comprising the nucleotide sequence of SEQ. ID. NO. 30 expressing an Aromatic prenyltransferase.

2. A method for making Tetrahydrocannabivarinic acid in yeast, the method comprising:
The method of claim 1, where the Cannabigerovarinic Acid is converted to Tetrahydrocannabivarinic acid with a ninth nucleotide sequence comprising the nucleotide sequence of SEQ. ID. NO.29 expressing THCA synthase.

3. A method for making Cannabidivarinic acid in yeast, the method comprising:
The method of claim 1, where the Cannabigerovarinic Acid is converted to Cannabidivarinic acid with a ninth nucleotide sequence comprising the nucleotide sequence of SEQ. ID. NO.28 expressing CBDA synthase.

4. The method of claim 1, the yeast further comprising K. marxianus.

5. The method of claim 1, the yeast further comprising S. cerevisiae.

6. The method of claim 1, the yeast further comprising Y. lipolytica.

7. The method of claim 1, the yeast further comprising Aspergillus oryzae.

8. The method of claim 1, the yeast further comprising Aspergillus nidulans.

9. The method of claim 1, the yeast further comprising Bacillus subtilis.

10. The method of claim 1, the yeast further comprising Corynebacterium glutamicum.

11. The method of claim 1, the yeast further comprising Synechococcus sp.

12. The method of claim 1, the yeast further comprising Komagataella pastoris.

13. The method of claim 2, the yeast further comprising K. marxianus.

14. The method of claim 2, the yeast further comprising S. cerevisiae.

15. The method of claim 2, the yeast further comprising Y. lipolytica.

16. The method of claim 2, the yeast further comprising Aspergillus oryzae.

17. The method of claim 2, the yeast further comprising Aspergillus nidulans.

18. The method of claim 2, the yeast further comprising Bacillus subtilis.

19. The method of claim 2, the yeast further comprising Corynebacterium glutamicum.

20. The method of claim 2, the yeast further comprising Synechococcus sp.

21. The method of claim 2, the yeast further comprising Komagataella pastoris.

22. The method of claim 3, the yeast further comprising K. marxianus.

23. The method of claim 3, the yeast further comprising S. cerevisiae.

24. The method of claim 3, the yeast further comprising Y. lipolytica.

25. The method of claim 3, the yeast further comprising Aspergillus oryzae.

26. The method of claim 3, the yeast further comprising Aspergillus nidulans.

27. The method of claim 3, the yeast further comprising Bacillus subtilis.

28. The method of claim 3, the yeast further comprising Corynebacterium glutamicum.

29. The method of claim 3, the yeast further comprising Synechococcus sp.

30. The method of claim 3, the yeast further comprising Komagataella pastoris.
